(19) United States
(12) Patent Application Publication
(10) Pub. No.: US 2024/0175869 A1
(43) Pub. Date: May 30, 2024

(54) CHARACTERIZING THE BINDING INTERACTIONS BETWEEN MUSK AND BMP RECEPTORS

(71) Applicant: Brown University, Providence, RI (US)

(72) Inventors: Justin R. Fallon, Providence, RI (US); Diego Jaime, Providence, RI (US); Mark Gad, Providence, RI (US)

(21) Appl. No.: 18/282,463

(22) PCT Filed: Mar. 18, 2022
(86) PCT No.: PCT/US2022/020990

§ 371 Date: Sep. 15, 2023

Related U.S. Application Data
(60) Provisional application No. 63/166,915, filed on Mar. 26, 2021.
(60) Provisional application No. 63/162,796, filed on Mar. 18, 2021.

(51) Int. Cl. G01N 33/564; A01K 67/0276; G01N 33/50

(52) U.S. Cl. CPC G01N 33/564; A01K 67/0276; G01N 33/5008; A01K 2227/105; G01N 2333/912; G01N 2800/2878

(57) ABSTRACT

Methods and compositions for increasing neurogenesis or muscle regeneration and for preventing or treating diseases, disorders or conditions associated with neurodegeneration or muscle regeneration, are provided herein.

### STATEMENT REGARDING FEDERALLY SPONSORED RESEARCH OR DEVELOPMENT

This invention was made with government support under grant number R21 NS112743 awarded by the National Institutes of Health. The government has certain rights in the invention.

### FIELD OF THE INVENTION

This disclosure generally relates to drugs for disorders of the nervous system for treating neurodegenerative disorders of the central nervous system, e.g., nootropic agents, cognition enhancers, drugs for treating Alzheimer's disease or other forms of dementia. The present disclosure relates to modulating certain interactions between MuSK and BMP or a BMP receptor I.

### BACKGROUND

Bone Morphogenetic Proteins (BMPs) are well-known to have important functions in all parts of the human body. Wang et al. (2014). BMP signaling regulates a range of cellular events including the activation of neural and muscle stem cells, neural plasticity, bone growth, sarcopenia, and tumorigenesis. Some BMP functions include bone and cartilage formation, vascular development, homeostasis, cancerous transformation, and intestinal epithelium formation. There remains the question of how the BMP signal is modulated in a tissue-specific manner. About twenty types of BMPs are available for signaling. These twenty BMPs are believed to signal through the same three Type 1 BMP receptors. Yadin, Knaus, & Müller (2016). How these signals and functions are regulated in a tissue-specific manner remains to be characterized.

BMP signaling has been difficult to harness therapeutically, because BMP signaling is used in many contexts, making specificity of targeting challenging. BMP2 was developed by Genetics Institute as a therapeutic promoter of bone growth. See, for example, Burkus et al., J. Spinal Disord. Tech., 15, 337 (2002). Using BMP2 was restricted because of unwanted side effects, such as stimulating tumor progression. See, for example, James et al., Tissue Eng. Part B, Rev. 22, 284 (2016).

MuSK is a receptor tyrosine kinase. The ectodomain of MuSK comprises 3 Ig-like domains and a CRD/Frizzled domain, while the intracellular domain contains a tyrosine kinase domain. See Hubbard & Gnanasambandan (2013). MuSK is expressed in skeletal muscle and other mammalian tissue. Garcia-Osta et al. (2006). MuSK has a vital role in forming neuromuscular junctions. When MuSK is not present in mice, these mice cannot form neuromuscular junctions. They die at birth. See Hubbard & Gnanasambandan (2013).

MuSK is a BMP co-receptor and binds to BMPs at the BMP Ig3 domain. A critical subset of BMP signaling is mediated by the ‘MuSK-BMP pathway.’ See Yilmaz et al. (2016); Fish & Fallon (2020). MuSK not only binds BMPs but also binds type 1 BMP receptors (BMPR1A and BMPR1B). The MuSK-BMP pathway is intrinsically much more selective than general BMP signaling because MuSK is expressed only in a subset of tissues and cells. MuSK binds to both BMPs and the Type I BMP receptors BMPR1a, BMR1b and Acvr1b (also known as ALK 3, 6 and 4, respectively). ALK is Anaplastic Lymphoma Kinase. Manipulation of the MuSK Ig3 domain, which is necessary for high affinity BMP4 binding, promotes adult hippocampal neurogenesis and muscle regeneration. See, WO 2020/214987 A1.

But there remains a need in the biomedical art for a better understanding of all the MuSK interactions and binding partners. Successful development of drugs that target (e.g., to modulate) the BMP pathway, would be facilitated by an improved understanding of MuSK interactions and binding partners, and the roles they play in biological events.

### SUMMARY

The invention provides compositions and methods for the selective manipulation of the Bone Morphogenetic Protein (BMP) pathway. The invention is based upon the observation that BMP receptors (ALK) bind to the Ig2 domain of Muscle-specific Kinase (MuSK). This specification identifies and characterizes interactions between BMP receptors and a BMP co-receptor, Muscle-specific Kinase (MuSK).

In one aspect, the invention provides several elements to the observation that BMP receptors (ALK) bind to the Ig2 domain of MuSK.

The inventors identified BMP receptors that bind MuSK. The inventors mapped the MuSK domains necessary to bind to BMPR1. MuSK-BMPR1 binding may be an important regulatory mechanism for BMP signaling.

The inventors mapped the MuSK domains necessary for binding to BMP receptors (ALK). The inventors designed and generated different His-tagged deletion constructs of the MuSK ectodomain, and isolated domains. The inventors used a solid-phase binding assay to quantitate the binding of BMPR1A and BMPR1b to different MuSK constructs and generate binding curves for each construct.

BMPR1a is also known as ALK3 and BMPR1b is also known as ALK6. BMPR1a and BMPR1b are BMP receptors that bind to MuSK although the binding domain was previously unknown. The inventors identified a single domain (Ig2) required for MuSK-BMPR1 binding. This domain is necessary and sufficient for binding BMPR1. This MuSK Ig2 domain is thus necessary and sufficient for binding to the BMP receptors. The inventors designed and expressed deletion constructs of MuSK were. An ELISA-like solid-phase binding assay was then used to assay the binding between these deletion constructs and ALK6. ΔIg2 MuSK showed little to no binding. This result shows the necessity of the Ig2 domain to MuSK binding to ALK6.

The inventors located where such binding happens in a human cell. The inventors assayed the binding of MuSK to BMP receptors under different pH conditions. They showed the MuSK binding to BMPR1 is pH-dependent by assaying the binding of MuSK to BMP receptors under different pH conditions. This characterization of the MuSK-BMPR1 interaction is useful in supporting a therapeutic development program targeting the MuSK-BMP pathway for Alzheimer's disease.

The inventors identified previously unrecognized splicing variants of MuSK. The inventors also showed that the alternative splicing of two ‘small’ 30 bp exons (5a and 5b) can regulate both the level of BMP receptor binding and the selectivity of MuSK for different BMP receptors. The protein in the Ig2 region encoded by these small exons bind to MuSK. Manipulating the small exons could provide increased selectivity of therapeutic modulation of BMP signaling.

In some embodiments, the disclosure provides a method for treating Alzheimer's disease, Muscle-specific Kinase Myasthenia Gravis (MuSK-MG) and other neurological disorders based upon modulating the MuSK Ig2 domain.

In some embodiments, the disclosure provides a method for treating muscle disorder (e.g., myocardial infarction, cardiomyopathy), or genetic diseases characterized by muscle wasting. In some embodiments, the subject being treated can be at risk of or afflicted with a disease or disorder associated with lung damage, including idiopathic pulmonary fibrosis (IPF), acute respiratory distress syndrome (ARDS), pneumonia, or certain infections, including viral infections including coronaviral infections such as COVID19.

Exemplary muscle disorders that can be treated by technologies of the present invention include, but are not limited to, Becker muscular dystrophy, Congenital muscular dystrophy, Distal muscular dystrophy, Duchenne muscular dystrophy, Emery-Dreifuss muscular dystrophy, Facioscapulohumeral muscular dystrophy, Limb-girdle muscular dystrophy, Myotonic muscular dystrophy, and Oculo-pharyngeal muscular dystrophy.

In some embodiments, enhancing muscle growth is used in treating a disease or disorder associated with muscle atrophy or muscle wasting. Muscle atrophy or muscle wasting may be observed with various diseases and conditions described, such as neuromuscular disorders, or direct or indirectly caused by prolonged inactivity, bed rest, hospitalization, aging, malnutrition, cancer cachexia, chronic inflammatory diseases, etc. Example chronic inflammatory diseases include rheumatoid arthritis, chronic heart failure, and chronic obstructive pulmonary disease (COPD).

In some embodiments, methods of the present invention can also be used when a subject needs enhanced muscle regeneration and muscle growth following surgery, trauma or prolonged immobilization (e.g., from bed-rest or casting). As muscle stem cell activity decreases with age, methods of the present invention can also prevent or reverse sarcopenia in patients otherwise healthy and could lead to significant improvements in quality of life and autonomy.

In some embodiments, the disclosure provides a method for treating Alzheimer's disease, MuSK-MG and other neurological or muscle disorders based upon modulating the alternative splicing of the 5a and 5b MuSK exons.

In some embodiments, the disclosure provides a diagnostic for MuSK-MG based on the binding of autoantibodies to the MuSK Ig2 domain.

In some embodiments, the disclosure provides a diagnostic for MuSK-MG based on the binding of autoantibodies to MuSK exons 5a and 5b.

In some embodiments, the disclosure provides a method for treating sarcopenia, cachexia and other muscle disorders based on the modulation of the MuSK Ig2 domain.

In some embodiments, the disclosure provides a method for treating sarcopenia, cachexia and other muscle disorders based on the modulation of the MuSK exons 5a and 5b.

In some embodiments, the disclosure provides a method for treating a subject suffering from one or more features of neurodegeneration, impaired cognition, or muscle disorder, the method comprising a step of: (a) increasing level or activity of a MuSK polypeptide lacking a functional Ig2 domain; or (b) reducing level or activity of a BMP receptor (ALK)-MuSK polypeptide complex, wherein the MuSK comprises a functional Ig2 domain.

In some embodiments, the disclosure provides a method for increasing neurogenesis, muscle regeneration and growth, the method comprising a step of (a) increasing level or activity of a MuSK polypeptide lacking a functional Ig2 domain; or (b) reducing level or activity of a BMP-MuSK polypeptide complex, wherein the MuSK comprises a functional Ig2 domain.

In some embodiments, the method further comprises the step of administering a pharmaceutical composition that comprises or delivers a MuSK Ig2 modulator agent.

In some embodiments, the method further comprises administering a pharmaceutical composition that increases the altered splicing of MuSK transcripts, wherein the alteration comprises skipping one or more exons in the MuSK Ig2 domain, wherein the skipped exon is exon 5a of MuSK Ig2 domain; or wherein the skipped exon is exon 5b of MuSK Ig2 domain; or wherein the skipped exons are exons 5a and 5b of MuSK Ig2 domain. The MuSK 5a and 5b exons are amenable to modulation with both therapeutic antibodies and antisense oligonucleotides. In another embodiment, the modulation is by the administration of an antisense oligonucleotide to induce exon skipping.

In some embodiments, the disclosure provides a method for inducing MuSK exon skipping by contacting a system comprising a population of MuSK primary transcripts with an oligonucleotide that binds to such primary transcripts so that skipping of one or both of exons 5a and 5b is increased.

In some embodiments, the disclosure provides a method for characterizing a MuSK Ig2 modulator agent, the method comprising one or more of these steps (a) assessing ability to reduce MuSK-Ig2-BMP receptor (ALK) complex formation; (b) assessing ability to alter splicing pattern of primary MuSK transcripts; (c) assessing ability to inhibit expression of transcript; (d) assessing the ability to increase expression of MuSK transcripts lacking a sequence encoding the Ig2 domain; (e) assessing the ability to increase level of MuSK polypeptide lacking functional Ig2; and (f) assessing the ability to impact characteristics of cells in a population.

In some embodiments, the disclosure provides a genetically modified mouse that comprises in its genome a sequence encoding the MuSK, wherein the sequence encoding MuSK does not include nucleotides within a region from (in 5′ to 3′ order) exon 5a to exon 5b; wherein the genetically modified mouse is not capable of expressing the full length MuSK transcript or producing full length MuSK protein.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1 is a schematic showing the process of BMP binding in a cell.

FIG. 2 is a schematic showing the full-length ectodomain and endodomain of MuSK. The ectodomain of MuSK comprises three Ig-like domains and a cysteine-rich CRD/Frizzled domain, while the intracellular domain contains a tyrosine kinase domain.

FIG. 3 is a schematic showing the deletion constructs that the inventors generated.

FIG. 4 is an electrophoresis gel confirming the presence of the full-length, ΔIg1, ΔIg2, and ΔIg1Ig2 constructs.

FIG. 5 is an electrophoresis gel confirming the Ig1, Ig2, Ig1-Ig2, and ACRD/Fz constructs.

FIG. 6 is an electrophoresis gel confirming the presence and expected size for the CRD/Fz, Δ5a5a′, and L83R constructs.

FIG. 7 is a line graph showing the data from a solid phase binding assay where MuSK was immobilized and incubated with ALK6.

FIG. 8 is a line graph showing that with the Ig2 domain necessary for binding, showing that the Ig1-Ig2 construct has a high-affinity binding to ALK6 and that the Ig1 and the Ig2 domain are both necessary for binding MuSK to ALK6.

FIG. 9 is a line graph showing that the Ig2 domain is sufficient to bind ALK6 using isolated Ig2 and Ig1 domains.

FIG. 10 is a line graph showing that MuSK binds strongly and with a high affinity to ALK6. Comparison of the ΔIg1 curve to the ΔIg2 curve indicated that the Ig1 domain is unnecessary for MuSK binding to ALK6.

FIG. 11 is a schematic showing the Ig1-Ig2-only constructs the MuSK ectodomain, where only the Ig1 domain and the Ig2 domain are present and that was His-tagged for purification.

FIG. 12 is a line graph showing that MuSK binds significantly to ALK6 under slightly acidic conditions using a solid-phase binding assay. The interaction between MuSK and ALK6 at pH 6 is highly favorable at this condition compared to pH 7.0.

FIG. 13 is a line graph showing that, when the Ig1 domain is deleted, MuSK shows no pH dependence, suggesting that the Ig1 domain is the reason MuSK binds differently to ALK6 at pH 6 and pH 7.

FIG. 14 is a schematic model of MuSK-ALK6 binding at the cell surface (pH 7.4) and the endosome compartment (pH 6.0). The first upper left panel shows MuSK as a dimer and BMP receptors canonically binding to BMPs. An event in the cell then triggers clathrin-mediated endocytosis (upper right panel). For the cell to initiate clathrin-mediated endocytosis, the accumulation of PIP2 and adaptor proteins is necessary. Clathrin-mediated endocytosis leads to form endosomes (bottom right panel). Endosomes have a slightly acidic pH compared to the extracellular environment of cells. BMP receptors type 1 favors binding MuSK at these conditions (bottom left panel). The lower pH causes the BMP to also dissociate from BMP receptors and bind to the BMP receptor.

FIG. 15 is an electrophoresis gel showing the detection of novel small exons (5a and 5b) in MuSK, thus demonstrating alternative splicing in the MuSK region containing alternatively spliced exons 5a and 5b. PCR of muscle (middle) and satellite cell (right) cDNA using primers (forward, exon 5; reverse exon 8). Note that three forms are detected (5a only; 5a+5b; no small exons).

FIG. 16 provides the amino acid sequence of the MuSK containing alternatively spliced exons 5a and 5b.

FIG. 17 shows alternatively splicing patterns of exons 5a and 5b from mouse soleus muscle.

### DETAILED DESCRIPTION OF THE INVENTION

### Industrial Applicability

The over twenty BMPs signal through only seven BMP receptors. The functions of BMPs are specific. So, how can so many different functions be derived from the same combinations of BMP and BMP receptors? Such questions make it interesting for researchers to identify all the co-receptors and regulators of the BMP signaling pathway.

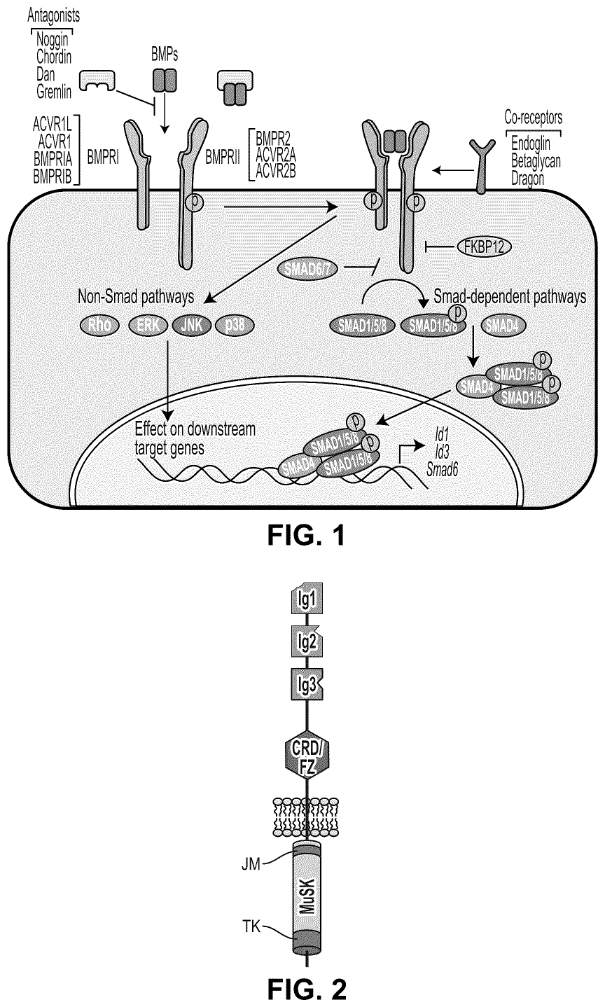

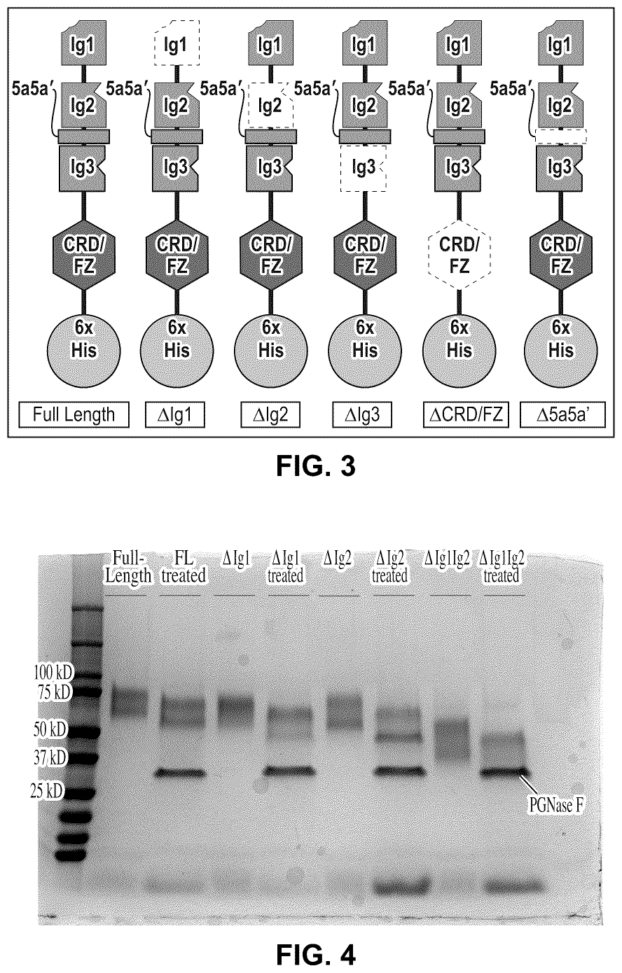

MuSK. MuSK is a receptor tyrosine kinase comprised extracellularly of three Ig and one CRD/Fz domain and an intracellular tyrosine domain (TK; FIG. 1). The best understood function of MuSK is at the neuromuscular junction (NMJ) where agrin-LRP4 binding to the Ig1 domain triggers MuSK TK activity and synapse differentiation (Kim et al., 2008; Zhang et al., 2008a).

The MuSK-BMP Pathway. The brain harbors neural stem cells (NSCs) that generate neurons and glial cells throughout life. Moreno-Jimenez et al. (2019); Steiner et al. (2019). BMPs regulate at least two important NSC decision points: (1) quiescence, where proliferating stem cells exit the cell cycle and return to replenish a reserve pool that can supply fresh stem cells; and (2) differentiation into mature progeny. Mira et al. (2010). The present disclosure contemplates that manipulating the BMP pathway in NSCs is an attractive target for regulating neurogenesis in the adult brain.

MuSK is also a BMP co-receptor that binds BMP and its receptors ALK3, 4 and 6, upregulates BMP signaling and shapes the composition of the transcriptional response in myogenic cells. See, Yilmaz et al., Sci. Signal., 9, ra87 (2016). This BMP signaling pathway neither regulates nor requires MuSK TK activity nor is it activated by agrin-LRP4. The MuSK Ig3 domain is necessary for high affinity BMP binding but is dispensable for agrin-LRP4 TK activation. The Ig3 domain is endogenously alternatively spliced, including in the brain. Garcia-Osta et al. (2006); Hesser et al. (1999). BMP signaling induces neural stem cell (NSC) quiescence and can inhibit integration of newborn neurons. The inventors found that restraining BMP drive by reducing MuSK-BMP signaling could increase neurogenesis.

Yilmaz et al., Sci. Signal., 9, ra87 (2016) discloses that the ‘Ig3’ domain of MuSK is required for high affinity binding of BMPs. The major species of MuSK expressed endogenously is full length. This Ig3 domain can be alternatively spliced endogenously, creating an isoform termed Δg3MuSK. This splicing entails the coordinated removal of exons 6 and 7 from the MuSK pre-mRNA. Regulating MuSK alternative splicing is a strategy for increasing neurogenesis in Alzheimer's disease. See WO 2020/214987 A1 (Brown University). MuSK is expressed in muscle and is upregulated during muscle regeneration. Data suggest that MuSK is implicated in BMP signaling in myogenesis. MuSK can act as a BMP co-receptor that binds BMP2, BMP4, and BMP7 and the Type I BMP receptors ALK3 and ALK6. See, for example, Yilmaz et al., Sci. Signal., 9, ra87 (2016), incorporated herein by reference. The Ig3 domain of MuSK is required for high-affinity binding to BMP. MuSK upregulates BMP signaling as measured by BMP4-dependent phosphorylation of SMAD1/5/8. MuSK-BMP signaling shapes the magnitude and composition of BMP-induced transcriptome in myoblasts and myotubes and this role is independent of any MuSK tyrosine kinase activity. MuSK is a BMP co-receptor that potentiates BMP signaling and regulates myogenic factors, such as myogenic factor 5 (Myf5), in immortalized myogenic cells.

Activated satellite cells express MuSK protein and disruption of MuSK-BMP signaling alters satellite cell proliferation in regenerating muscle in vivo. Data included provide information on the role of the MuSK-BMP pathway in satellite cells and muscle regeneration. These data suggest that the MuSK-BMP pathway plays a key role, and the present disclosure provides an insight that targeting the MuSK-BMP pathway is more selective than targeting BMP-wide activity, at least because MuSK expression is far more restricted than that of the BMP system. Data provided also teach that targeting MuSK-BMP pathway enhances muscle growth. In some embodiments, muscle growth occurs in, e.g., uninjured tissues.

Since the MuSK ectodomain is exposed on the surface of the cell it is amenable to modulation by therapeutic antibodies or other agents that act extracellularly.

The MuSK Ig2 domain and the two small exons emerges as target for modulating the MuSK-BMP pathway to treat disorders such as Alzheimer's, stroke, PTSD and treatment-resistant depression.

The MuSK Ig2 domain could also be a target of autoantibodies that mediate MuSK-dependent myasthenia gravis. Myasthenia gravis (MG) is a chronic autoimmune neuromuscular disease characterized by weakness of the skeletal muscles. Jayam Trouth et al. (2012). Clinical research has shown that myasthenia gravis patients can sometimes have antibodies against MuSK. Guptill, Sanders, & Evoli A (2011). These anti-MuSK antibodies are present in many patients that do not present with antibodies against acetylcholine receptors. Guptill, Sanders, & Evoli A (2011). MuSK-dependent myasthenia gravis (MuSK-MG) may be caused by autoantibodies directed against the MuSK Ig1 domain, inhibiting agrin-LRP4 signaling. The present disclosure teaches that the autoantibodies present in these patients are directed against the MuSK Ig2 domain, which could be inhibiting MuSK-BMP signaling and therefore responsible for the muscle atrophy observed in these patients. Findings provided by the present disclosure have potential value both for diagnostics and therapeutics. For example, disrupting interaction between such autoantibodies and MuSK Ig2 could be an effective therapeutic strategy. Or and detection of autoantibodies that bind MuSK Ig2 could be useful in diagnosing Myasthenia Gravis.

### Definitions

For convenience, the meaning of some terms and phrases used in the specification, examples, and appended claims, are listed below. Unless stated otherwise or implicit from context, these terms and phrases shall have the meanings below. These definitions aid in describing particular embodiments but are not intended to limit the claimed invention. Unless otherwise defined, all technical and scientific terms have the same meaning as commonly understood by a person having ordinary skill in the art to which this invention belongs. A term's meaning provided in this specification shall prevail if any apparent discrepancy arises between the meaning of a definition provided in this specification and the term's use in the biomedical art.

About, when used referring to a value, refers to a value that is similar, in context to the referenced value. In general, those skilled in the biomedical art, appreciate the degree of variance encompassed by about in that context.

Administration refers to the administration of a pharmaceutical composition to a subject or system, to achieve delivery of an agent, e.g., an agonizing agent that is, or is included in or otherwise delivered by, the composition. Persons having ordinary skill in the biomedical art know a variety of routes that can be used for administration to a subject, e.g., a human. Administration can be ocular, oral, buccal, dermal, which can be or comprise, one or more of topical to the dermis, intradermal, interdermal, transdermal, etc., enteral, intra-arterial, intradermal, intragastric, intramedullary, intramuscular, intranasal, intraperitoneal, intrathecal, intravenous, intraventricular, within a specific organ, e.g., intrahepatic, mucosal, nasal, oral, rectal, subcutaneous, sublingual, topical, tracheal, e.g., by intratracheal instillation, vaginal, vitreal, etc. An agent, e.g., an agonizing agent is delivered to the central nervous system (CNS), e.g., delivered via intracerebroventricular administration. Administration can involve only a single dose. Administration can involve application of a fixed number of doses. Administration can involve dosing that is intermittent, e.g., a plurality of doses separated in time or periodic, e.g., individual doses separated by a common period of time dosing. Administration can involve continuous dosing, e.g., perfusion for at least a selected period of time.

Agent is used by persons having ordinary skill in the biomedical art to refer to a compound or entity of any chemical class including, a polypeptide, nucleic acid, saccharide, lipid, small molecule, metal, or combination or complex thereof. Persons having ordinary skill in the biomedical art know that the term agent refers to an entity that is or comprises a cell or organism, or a fraction, extract, or component thereof. The term can refer to a natural product because it is found in or is obtained from nature. The term can refer to one or more entities that is man-made in that it is designed, engineered, or produced through action of the hand of man or is not found in nature. An agent can be used in isolated or pure form; an agent can be used in crude form. Potential agents can be provided as collections or libraries, that can be screened to identify or characterize active agents within them. The term agent can refer to a compound or entity that is or comprises a polymer. The term can refer to a compound or entity that comprises one or more polymeric moieties. The term agent can refer to a compound or entity that is not a polymer or is substantially free of any polymer or of one or more particular polymeric moieties. the term can refer to a compound or entity that lacks or is substantially free of any polymeric moiety.

Agonist is used by persons having ordinary skill in the biomedical art to refer to an agent (i.e., an agonizing agent), condition, or event whose presence, level, degree, type, or form correlates with increased level of activity of another agent, i.e., the agonized agent or the target agent. An agonist can be or include an agent of any chemical class including, small molecules, polypeptides, nucleic acids, carbohydrates, lipids, metals, or any other entity that shows the activating activity. An agonist can be direct (in which case it exerts its influence directly upon its target); an agonist can be indirect (in which case it exerts its influence by other than binding to its target; e.g., by interacting with a regulator of the target, so level or activity of the target is altered. An agonist is a binding agent that is a protein, e.g., an antibody or a nucleic acid, e.g., an antisense oligonucleotide that binds a target, e.g., a protein or nucleic acid so that level, form, or activity of the target is altered. the altered level, form or activity is an increased level of altered protein expressed from the target nucleic acid sequence. Persons having ordinary skill in the biomedical art know that an agonizing agent can bind to and potentially agonize a binding target, which binding causes an increase in level or activity of a further agonized target. An agonizing agent that binds to a nucleic acid target can alter level or activity of that target. An agonizing agent can agonize an activity of that nucleic acid target, e.g., by increasing its modification, splicing, 5′ cap formation, or 3′ end formation, transport, or translation, etc., so that a level of a desired product, e.g., mRNA, is increased or can agonize a downstream target, such as a polypeptide encoded by such nucleic acid target. An agonizing agent can be or comprise an oligonucleotide that binds to a primary transcript and alters its splicing pattern so that level or activity of a particular spliced form, e.g., mature mRNA is increased, which can achieve increased level of a product, e.g., a polypeptide that is or is encoded by such particular spliced form.

Agonist therapy is used by persons having ordinary skill in the biomedical art to refer to administration of an agonist that agonizes a particular target of interest to achieve a desired therapeutic effect. Agonist therapy involves administering a single dose of an agonist. Agonist therapy involves administering several doses of an agonist. Agonist therapy involves administering an agonist according to a dosing regimen known or expected to achieve the therapeutic effect, because such result was established to a designated degree of statistical confidence, e.g., through administration to a relevant population. Agonist therapy involves delivery of agonizing agent as described in this specification. An agonizing agent can be or comprise a binding agent that is a protein, e.g., an antibody or a nucleic acid, e.g., an antisense oligonucleotide that binds a target, e.g., a protein or nucleic acid, so that level, form, or activity of the target is altered. An agonizing agent can bind to and potentially agonize a binding target, which binding causes an increase in level or activity of a further agonized target. An agonizing agent that binds to a nucleic acid target can alter level or activity of that target. An agonizing agent can agonize an activity of that nucleic acid target, e.g., by increasing its modification, splicing, 5′ cap formation, or 3′ end formation, transport, or translation, etc., so that a level of a desired product, e.g., mRNA, is generated or can agonize a downstream target, such as a polypeptide encoded by such nucleic acid target. An agonizing agent can be or comprise an oligonucleotide that binds to a primary transcript and alters its splicing pattern so that level or activity of a particular spliced form, e.g., mature mRNA is generated, which can achieve increased level of a product, e.g., a polypeptide that is or is encoded by such particular spliced form.

Antagonist is used by persons having ordinary skill in the biomedical art to refer to an agent (i.e., an antagonizing agent), condition, or event whose presence, level, degree, type, or form correlates with decreased level or activity of another agent (i.e., the inhibited agent, or target). An antagonist can be or include an agent of any chemical class including, small molecules, polypeptides, nucleic acids, carbohydrates, lipids, metals, or any other entity that shows the relevant inhibitory activity. An antagonist can be direct (in which case it exerts its influence directly upon its target; an antagonist can be indirect (in which case it exerts its influence by other than binding to its target; e.g., by interacting with a regulator of the target, so that level or activity of the target is altered. An antagonist is binding agent that is a protein, e.g., an antibody or a nucleic acid, e.g., an antisense oligonucleotide that binds a target, e.g., a protein or nucleic acid so that the level, form, or activity of the target is altered. the altered level, form or activity is a decreased level of altered protein expressed from the target nucleic acid sequence. Persons having ordinary skill in the biomedical art know that, an antagonizing agent can bind to and potentially antagonize a binding target, which binding causes a decrease in level or activity of a further antagonized target. An antagonizing agent that binds to a nucleic acid target can alter level or activity of that target. An antagonizing agent can antagonize an activity of that nucleic acid target, e.g., by decreasing its modification, splicing, 5′ cap formation, or 3′ end formation, transport, or translation, etc., so that a level of an undesired product, e.g., mRNA, is suppressed or can antagonize a downstream target, such as a polypeptide encoded by such nucleic acid target. An antagonizing agent can be or comprise an oligonucleotide that binds to a primary transcript and alters its splicing pattern so that level or activity of a particular spliced form, e.g., mature mRNA is suppressed, which can achieve decreased level of a product, e.g., a polypeptide that is or is encoded by such particular spliced form.

Antibody agent is used by persons having ordinary skill in the biomedical art to refer to an agent that specifically binds to a particular antigen, e.g., that can be or comprise an epitope of a protein of interest, e.g., a MuSK protein. The term encompasses any polypeptide or polypeptide complex that includes immunoglobulin structural elements sufficient to confer specific binding. Exemplary antibody agents include monoclonal antibodies or polyclonal antibodies. An antibody agent can include one or more constant region sequences characteristic of mouse, rabbit, primate, or human antibodies. An antibody agent can include one or more sequence elements are humanized, primatized, chimeric, etc., as known in the biomedical art. The term antibody agent can refer to one or more of the biomedical art-known or developed constructs or formats for utilizing antibody structural and functional features in alternative presentation. An antibody agent used under this invention is in a format selected from, but not limited to, intact IgA, IgG, IgE or IgM antibodies; bi- or multi-specific antibodies, e.g., Zybodies®, etc.; antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies, e.g., shark single domain antibodies such as IgNAR or fragments thereof; cameloid antibodies; masked antibodies, e.g., Probodies®; Small Modular ImmunoPharmaceuticals (SMIPs™); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®. An antibody can lack a covalent modification, e.g., attachment of a glycan that it would have if produced naturally. An antibody can contain a covalent modification, e.g., attachment of a glycan, a payload, e.g., a detectable moiety, a therapeutic moiety, a catalytic moiety, etc., or other pendant group, e.g., poly-ethylene glycol, etc. An antibody agent can be or can comprise a polypeptide whose amino acid sequence includes one or more structural elements recognized by persons having ordinary skill in the biomedical art as a complementarity determining region (CDR). An antibody agent can be or can comprise a polypeptide whose amino acid sequence includes at least one CDR, e.g., at least one heavy chain CDR or at least one light chain CDR substantially identical to one found in a reference antibody. An included CDR can substantially be identical to a reference CDR because it is identical in sequence or contains between 1-5 amino acid substitutions as compared with the reference CDR. An included CDR can be substantially identical to a reference CDR because it shows at least 85%, 86%, 87%, 88%, 89%, 90%, 91%, 92%, 93%, 94%, 95%, 96%, 97%, 98%, 99%, or 100% sequence identity with the reference CDR. An included CDR can be substantially identical to a reference CDR because it shows at least 96%, 96%, 97%, 98%, 99%, or 100% sequence identity with the reference CDR. An included CDR can be substantially identical to a reference CDR in that at least one amino acid within the included CDR is deleted, added, or substituted as compared with the reference CDR but the included CDR has an amino acid sequence that is otherwise identical with that of the reference CDR. An included CDR can be substantially identical to a reference CDR in that 1-5 amino acids within the included CDR are deleted, added, or substituted as compared with the reference CDR but the included CDR has an amino acid sequence that is otherwise identical to the reference CDR. An included CDR can be substantially identical to a reference CDR in that at least one amino acid within the included CDR is substituted as compared with the reference CDR but the included CDR has an amino acid sequence that is otherwise identical with that of the reference CDR. An included CDR can be substantially identical to a reference CDR in that 1-5 amino acids within the included CDR are deleted, added, or substituted as compared with the reference CDR but the included CDR has an amino acid sequence that is otherwise identical to the reference CDR. An antibody agent is or comprises a polypeptide whose amino acid sequence includes structural elements recognized by persons having ordinary skill in the biomedical art as an immunoglobulin variable domain. An antibody agent is a polypeptide protein having a binding domain which is homologous or largely homologous to an immunoglobulin-binding domain.

Antibody is used by persons having ordinary skill in the biomedical art to refer to an immunoglobulin or a derivative thereof containing an immunoglobulin domain capable of binding to an antigen, e.g., that can be or comprise an epitope of a protein of interest, e.g., a MuSK protein. The antibody can be of any species, e.g., human, rodent, rabbit, goat, chicken, etc. The antibody can be a member of any immunoglobulin class, including any of the human classes: IgG, IgM, IgA, IgD, and IgE, or subclasses thereof such as IgG1, IgG2, etc. The antibody can be a fragment such as a Fab′, F(ab′)2, scFv (single-chain variable) or other fragment that retains an antigen binding site, or a recombinantly produced scFv fragment, including recombinantly produced fragments. See, e.g., Allen (2002), and references therein. The antibody can be monovalent, bivalent or multivalent. The antibody can be a chimeric or humanized antibody in which, a variable domain of rodent origin is fused to a constant domain of human origin, thus retaining the specificity of the rodent antibody. The domain of human origin need not originate directly from a human in the sense that it is first synthesized in a human. Instead, human domains can be generated in rodents whose genome incorporates human immunoglobulin genes. See, e.g., Vaughan et al. (1998). The antibody can be partially or completely humanized. An antibody can be polyclonal or monoclonal, though for this invention monoclonal antibodies are generally preferred. Methods for producing antibodies that specifically bind to virtually any molecule of interest are known in the biomedical art. monoclonal or polyclonal antibodies can be purified from blood or ascites fluid of an animal that produces the antibody, e.g., after natural exposure to or immunization with the molecule or an antigenic fragment thereof, can be produced using recombinant techniques in cell culture or transgenic organisms, or can be made by chemical synthesis. the antibody can act as an antagonist, e.g., by binding to a target antigen, resulting in a decreased level or activity of the antigen. the antibody can act as an agonist, e.g., by binding to a target antigen, resulting in an increased level or increased activity of the antigen.

Antisense is used by persons having ordinary skill in the biomedical art to refer to a nucleic acid whose nucleotide sequence is complementary to part or all of a sequence found in a coding strand nucleic acid. Typically, a coding strand nucleic acid is one whose sequence includes part or all of an open reading frame or other stretch of residues that encodes part or all of a polypeptide. The term antisense can particularly refer to an oligonucleotide that binds specifically to a coding strand (i.e., to a target sequence within such coding strand. A coding strand can include both coding and non-coding sequences, e.g., to give but one example, can be a transcript, such as a primary transcript. that includes both intron and exon sequences. Persons having ordinary skill in the biomedical art, reading this disclosure, know that, an oligonucleotide can be considered or called an antisense oligonucleotide when some or all of its sequence is complementary to non-coding portions of its target strand. An antisense oligonucleotide binds to coding sequences in a target sense strand; an antisense oligonucleotide binds to non-coding sequences in a target coding strand. An antisense oligonucleotide binds to both coding and non-coding sequences in a target coding strand. An antisense oligonucleotide is characterized in that, when bound to its target sequence in a coding strand, e.g., a transcript, it alters post-transcriptional processing, e.g., one or more of modification, splicing, 5′ cap formation, or 3′ end formation, 5′ cap formation, or 3′ end formation, transport, or translation of such coding strand. An antisense oligonucleotide can alter splicing of its target coding strand, An antisense-coding strand complex is or can be degraded, e.g., by RNase H.

Approximately or about, when used referring to a number, includes numbers that fall within a range of 5%, 10%, 15%, or 20% in either direction (greater than or less than of the number unless otherwise stated or otherwise evident from the context.

Binding agent is used by persons having ordinary skill in the biomedical art to refer to any entity that binds to a target of interest as described in this specification. A binding agent of interest can bind specifically with its target because it discriminates its target from other potential binding partners in a particular interaction context. A binding agent can be or comprise an entity of any chemical class, e.g., polymer, non-polymer, small molecule, polypeptide, carbohydrate, lipid, nucleic acid, etc. A binding agent is a single chemical entity. A binding agent is a complex of two or more discrete chemical entities associated with one another under conditions by non-covalent interactions. persons having ordinary skill in the biomedical art know that a binding agent can comprise a generic binding moiety, e.g., one of biotin/avidin/streptavidin or a class-specific antibody and a specific binding moiety, e.g., an antibody or aptamers with a particular molecular target linked to the partner of the generic biding moiety. such an approach can permit modular assembly of several binding agents through linkage of different specific binding moieties with the same generic binding moiety partner. binding agents are or comprise polypeptides, including, e.g., antibodies or antibody fragments. binding agents are or comprise small molecules. binding agents are or comprise nucleic acids, e.g., antisense oligonucleotides. binding agents are aptamers. binding agents are polymers; binding agents are not polymers. binding agents are non-polymeric because they lack polymeric moieties. binding agents are or comprise carbohydrates. binding agents are or comprise lectins. binding agents are or comprise peptidomimetics. binding agents are or comprise scaffold proteins. binding agents are or comprise mimeotopes. Binding agents can be or can comprise stapled peptides. Binding agents can be or can comprise nucleic acids, such as DNA or RNA, e.g., antisense oligonucleotides.

BMP receptors are known to persons having ordinary skill in the biomedical art. BMP receptors (BMPRs) are serine/threonine kinase receptors, composed of a short extracellular domain with 10-12 cysteine residues, a single transmembrane domain, and the intracellular serine/threonine kinase domain. Bragdon et al. (2011) BMP receptors have two types: BMP receptors Type 1 (BMPR1) and BMP receptors type 2 (BMPR2). See Sanchez-Duffhues et al. (2020). BMP receptors are limited in number. Researchers identified five BMP type I receptors (BRI): ALK1 (Acvrl1), ALK2 (ActRI), ALK3 (BRIa), ALK4 (ActRIb) and ALK6 (BRIb); and three types II receptors: BRII, ActRIIa, and ActRIIb. See Bragdon et al. (2011). BMPs bind to combinations of BMP receptor type 1 and type 2. BMP has a canonical and a non-canonical signaling pathway. In the canonical pathway, BMP initiates the signaling by interacting with the BMP receptors to form a heterotetrameric complex comprised of two dimers of type I and type II. See Wang et al. (2014)). After this complex is formed, the type II receptors phosphorylate the type I receptors in the GS domain. After the type I receptor is phosphorylated, the type I receptor can phosphorylate downstream SMAD molecules, known as R-SMADs. The R-SMADs associated with BMP signaling are Smad1, Smad5, and Smad8. Wang et al. (2014). These Smads then bind to Smad4, and the entire complex is translocated inside the nucleus. Wang et al. (2014). The Smad complex acts as a transcription factor that can either activate or deactivate gene regulation Wang et al. (2014). FIG. 1 illustrates the process of BMP binding.

Bone Morphogenetic Proteins (BMPs) are known to persons having ordinary skill in the biomedical art as being essential molecules in a mammalian body, including a human body. BMPs are a group of signaling molecules that belong to the Transforming Growth Factor-β (TGF-β) superfamily of proteins. See Bragdon et al. (2011). More than 20 BMPs were discovered, making BMPS the Transforming Growth Factor-β (TGF-β) superfamily of proteins. See Bragdon eta. (2011). While BMPs were originally identified as molecules that assist with bone development, BMPs were later shown to have a role in many developmental functions of the body. See Wang et al. (2014).

Characteristic sequence element is used by persons having ordinary skill in the biomedical art to refer to a sequence element found in a polymer, e.g., in a polypeptide or nucleic acid that is a characteristic portion of that polymer. The presence of a characteristic sequence element correlates with presence or level of a particular activity or property of the polymer. presence or absence of a characteristic sequence element defines a particular polymer as a member or not a member of a particular family or group of such polymers. A characteristic sequence element typically comprises at least two monomers, e.g., amino acids or nucleotides. A characteristic sequence element includes contiguously-linked monomers. A characteristic sequence element includes at least first and second stretches of contiguous monomers spaced apart by one or more spacer regions whose length might vary across polymers that share the sequence element.

Complementary is used by persons having ordinary skill in the biomedical art to refer to the capacity for precise pairing between particular bases, nucleosides, nucleotides or nucleic acids. Adenine (A) and uridine (U) are complementary; adenine (A) and thymidine (T) are complementary; and guanine (G) and cytosine (C), are complementary and are referred to in the biomedical art as Watson-Crick base pairings. If a nucleotide at a particular position of a first nucleic acid sequence is complementary to a nucleotide located opposite in a second nucleic acid sequence when the strands are aligned in anti-parallel orientation, the nucleotides (nt) form a complementary base pair, and the nucleic acids are complementary at that position. The percent complementarity of a first nucleic acid to a second nucleic acid can be evaluated by aligning them in antiparallel orientation for maximum complementarity over a window of evaluation, determining the total number of nt in both strands that form complementary base pairs within the window, dividing by the total number of nucleotides within the window, and multiplying by 100. AAAAAAAA and TTTGTTAT are 75% complementary because there are twelve nucleotides in complementary base pairs out of sixteen nucleotides. When computing the number of complementary nt needed to achieve a particular % complementarity, fractions are rounded to the nearest whole number. A position occupied by non-complementary nucleotides constitutes a mismatch, i.e., the position is occupied by a non-complementary base pair. A window of evaluation can have the length described in this specification for duplex portions or target portions. Complementary sequences include base-pairing of a polynucleotide comprising a first nucleotide sequence to a polynucleotide comprising a second nucleotide sequence over the entire length of both nucleotide sequences, if the same length, or over the entire length of the shorter sequence, if different lengths. Such sequences have perfectly complementary (100% complementarity) regarding each other. Nucleic acids that are at least 70% complementary over a window of evaluation are considered substantially complementary over that window. Complementary nucleic acids can be at least 75%, at least 80%, at least 85%, at least 90%, or at least 95% complementary over the window of evaluation. Where a first sequence is called substantially complementary regarding a second sequence described in this specification, the two sequences can be perfectly complementary or they can comprise one or more unmatched bases upon hybridization, e.g., up to about 5%, 10%, 15%, 20%, or 25% unmatched bases upon hybridization, e.g., 1, 2, 3, 4, 5, or 6 mismatched base pairs upon hybridization for a duplex up to 30 base pairs, while retaining the ability to hybridize under the conditions most relevant to their intended use. When two oligonucleotides form, upon hybridization, one or more single stranded overhangs, such overhangs are not regarded as mismatches or unpaired nucleotides regarding determining percent complementarity. the two strands of a dsRNA comprising one oligonucleotide twenty-one nucleotides in length and another oligonucleotide twenty-three nucleotides in length, wherein the longer oligonucleotide comprises a sequence of twenty-one nucleotides that is perfectly complementary to the shorter oligonucleotide and a two-nucleotide overhang, can be called perfectly complementary. Complementary sequences can include one or more non-Watson-Crick base pairs or base pairs formed from non-natural and other modified nucleotides, in so far as the requirements regarding their ability to hybridize are fulfilled. Such non-Watson-Crick base pairs include G:U Wobble or Hoogsteen base pairing. Persons having ordinary skill in the biomedical art know that guanine, cytosine, adenine, and uracil can be replaced by other bases without substantially altering the base pairing properties of a polynucleotide comprising a nucleotide bearing such bases, according to the wobble rules. See, e.g., Murphy & Ramakrishnan (2004). A nucleotide comprising inosine as its base can base pair with nucleotides containing adenine, cytosine, or uracil. Nucleotides containing uracil, guanine, or adenine can be replaced in the nucleotide sequences of an inhibitory RNA described in this specification by a nucleotide containing, e.g., inosine. The terms complementary, perfectly complementary, and substantially complementary can be used regarding the base matching between any two nucleic acids, e.g., the base matching between the sense strand and the antisense strand of a double stranded nucleic acid, or portion thereof. Persons having ordinary skill in the biomedical art know that the term hybridize refers to the interaction between two nucleic acid sequences, which can be part of the same nucleic acid molecule or can be or can include parts of different nucleic acid molecules comprising or consisting of complementary portions, such that a duplex structure, i.e., an intramolecular or intermolecular duplex, is formed that is stable under the particular conditions of interest.

Combination therapy is used by persons having ordinary skill in the biomedical art to refer to those situations in which a subject is simultaneously exposed to two or more therapeutic regimens, e.g., two or more therapeutic agents. the two or more regimens can be administered simultaneously; such regimens can be administered sequentially e.g., all doses of a first regimen are administered before administration of any doses of a second regimen. Such agents are administered in overlapping dosing regimens. administration of combination therapy may involve administration of one or more agents or modalities to a subject receiving the other agents or modalities in the combination. For clarity, combination therapy does not require that individual agents be administered together in a single composition, or even necessarily at the same time, although two or more agents, or active moieties thereof, can be administered together in a combination composition, or even in a combination compound, e.g., as part of a single chemical complex or covalent entity.

Comparable is used by persons having ordinary skill in the biomedical art to refer to two or more agents, entities, situations, sets of conditions, etc., that may not be identical to one another but that are sufficiently similar to permit comparison there between so that persons having ordinary skill in the biomedical art know that conclusions may reasonably be drawn based on differences or similarities observed. Comparable sets of conditions, circumstances, individuals, or populations are characterized by a plurality of substantially identical features and one or a few varied features. Persons having ordinary skill in the biomedical art know in context what identity is required in any given circumstance for two or more such agents, entities, situations, sets of conditions, etc. to be considered comparable. Persons having ordinary skill in the biomedical art know that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features varied.

Comprises and comprising refer to elements, components, or steps in a non-exclusive manner, indicating that the referenced elements, components, or steps can be present, used, or combined with other elements, components, or steps. The singular terms a, an, and the include plural referents unless context indicates otherwise. Similarly, the inclusive term or should cover the term and unless the context indicates otherwise. The abbreviation e.g. means a non-limiting example and is synonymous with the term for example.

Domain is used by persons having ordinary skill in the biomedical art to refer to a section or portion of an entity. A domain is associated with a particular structural or functional feature of the entity so that, when the domain is physically separated from the rest of its parent entity, it substantially or entirely retains the particular structural or functional feature. A domain can be or include a portion of an entity that, when separated from that (parent) entity and linked with a different (recipient) entity, substantially retains or imparts on the recipient entity one or more structural or functional features that characterized it in the parent entity. A domain is a section or portion of a molecule, e.g., a small molecule, carbohydrate, lipid, nucleic acid, or polypeptide. A domain is a section of a polypeptide, e.g., the Ig2 domain of a MuSK protein. A domain can be characterized by a particular structural element, e.g., a particular amino acid sequence or sequence motif, alpha-helix character, beta-sheet character, coiled-coil character, random coil character, etc., or by a particular functional feature, e.g., binding activity, enzymatic activity, folding activity, signaling activity, etc.

Dosing regimen is used by persons having ordinary skill in the biomedical art to refer to a set of unit doses (typically more than one) that are administered individually to a subject, typically separated by periods of time. A given therapeutic agent has a recommended dosing regimen, which may involve one or more doses. A dosing regimen comprises a plurality of doses each of which is separated in time from other doses. individual doses are separated from one another by a time period of the same length; a dosing regimen comprises a plurality of doses and at least two different time periods separating individual doses. All doses within a dosing regimen are of the same unit dose amount. different doses within a dosing regimen are of different amounts. A dosing regimen comprises a first dose in a first dose amount, followed by one or more additional doses in a second dose amount different from the first dose amount. A dosing regimen comprises a first dose in a first dose amount, followed by one or more additional doses in a second dose amount same as the first dose amount. A dosing regimen is correlated with a desired or beneficial outcome when administered across a relevant population, i.e., is a therapeutic dosing regimen.

Engineered refers to the aspect of having been manipulated by the hand of man. An antibody, antibody reagent, antigen-binding portion thereof, CAR or bispecific antibody is considered to be engineered when the sequence of the antibody, antibody reagent, antigen-binding portion thereof, CAR or bispecific antibody is manipulated by the hand of man to differ from the sequence of an antibody as it exists in nature. In the biomedical art, progeny and copies of an engineered polynucleotide or polypeptide are typically still called engineered even though the actual manipulation was performed on a prior entity.

Expression of a nucleic acid sequence is used by persons having ordinary skill in the biomedical art to refer to one or more of these events: (1) production of an RNA template from a DNA sequence, e.g., by transcription; (2) processing of an RNA transcript, e.g., by splicing, editing, 5′ cap formation, or 3′ end formation; (3) transport of an RNA transcript, e.g., from nucleus to cytoplasm; or (4) translation of an RNA into a polypeptide or protein; or (4) post-translational modification of a polypeptide or protein.

Fragment of a material or entity is used by persons having ordinary skill in the biomedical art to refer to a structure that includes a discrete portion of the whole but lacks one or more moieties found in the whole. A fragment consists of such a discrete portion. A fragment consists of or comprises a characteristic structural element or moiety found in the whole. A polymer fragment comprises or consists of monomeric units, i.e., residues, as found in the whole polymer. A polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units, i.e., residues, found in the whole polymer. The whole material or entity may be called the parent of the fragment.

Gene is used by persons having ordinary skill in the biomedical art to refer to a DNA sequence in a chromosome that codes for a product, e.g., an RNA product or a polypeptide product. A gene includes coding sequence (i.e., sequence that encodes a particular product; a gene includes non-coding sequence. a gene can include both coding, i.e., exonic, sequences and non-coding, i.e., intronic, sequences. A gene can include one or more regulatory elements that can control or impact one or more aspects of gene expression, e.g., cell-type-specific expression, inducible expression, etc.

Gene product or expression product is used by persons having ordinary skill in the biomedical art to refer to an RNA transcribed from the gene (pre- or post-processing or a polypeptide (pre-modification or post-modification) encoded by an RNA transcribed from the gene. A gene product can be or comprise a particular processed form of an RNA transcript, e.g., a particular edited form, a particular splice form, a particular capped form, etc.

Homology is used by persons having ordinary skill in the biomedical art to refer to the overall relatedness between polymeric molecules, e.g., between nucleic acid molecules, e.g., DNA molecules or RNA molecules or between polypeptide molecules. Polymeric molecules are considered to be homologous to one another if their sequences are at least 25%, 30%, 35%, 40%, 45%, 50%, 55%, 60%, 65%, 70%, 75%, 80%, 85%, 90%, 95%, or 99% identical. polymeric molecules are considered to be homologous to one another if their sequences are at least 25%, 30%, 35%, 40%, 45%, 50%, 55%, 60%, 65%, 70%, 75%, 80%, 85%, 90%, 95%, or 99% similar.

Identity is used by persons having ordinary skill in the biomedical art to refer to the overall relatedness between polymeric molecules, e.g., between nucleic acid molecules, e.g., DNA molecules or RNA molecules or between polypeptide molecules. Polymeric molecules are considered to be substantially identical to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences can be performed by aligning the two sequences for optimal comparison purposes, e.g., gaps can be introduced in one or both of a first and a second sequences for optimal alignment and non-identical sequences can be disregarded for comparison. The length of a sequence aligned for comparison can be at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue, e.g., nucleotide or amino acid as the corresponding position in the second sequence, then the molecules are identical at that position. The percent identity between the two sequences is a function of the number of identical positions shared by the sequences, considering the number of gaps, and the length of each gap, which needs to be introduced for optimal alignment of the two sequences. The comparison of sequences and determination of percent identity between two sequences can be accomplished using a mathematical algorithm. The percent identity between two nucleotide sequences can be determined using the algorithm of Meyers and Miller (1989), which was incorporated into the ALIGN program (version 2.0). The nucleic acid sequence comparisons can be made with the ALIGN program use a PAM120 weight residue table, a gap length penalty of 12 and a gap penalty of 4. The percent identity between two nucleotide sequences can be determined using the GAP program in the GCG software package using an NWSgapdna.CMP matrix.

Improve, increase, inhibit,’ reduce, or grammatical equivalents thereof are used by persons having ordinary skill in the biomedical art to refer to indicate values relative to a baseline or other reference measurement. An appropriate reference measurement can be or comprise a measurement in a particular system, e.g., in a single individual, a single cell, or cell population under otherwise comparable conditions absent presence of, e.g., before or after a particular agent or treatment, or in presence of an appropriate reference agent, e.g., a positive control agent or a negative control agent. An appropriate reference measurement can be or comprise a measurement in comparable system known or expected to respond in a particular way, in presence of the agent or treatment. Persons having ordinary skill in the biomedical art know that an improvement, increase, reduction, etc. typically refers to a statistically significant change. Persons having ordinary skill in the biomedical art know from context what magnitude of change can be relevant. A change can be a fold change, i.e., so that a changed value is a 1.1, 1.2, 2, 3, 4, 5, 6, 7, 8, 9, 10, 15, 20, 30, 40, 50 or more, e.g., 500, 1000 times (including all integers and decimal points in between and above 1, e.g., 1.5, 1.6, 1.7. 1.8, etc.-fold difference relative to the relevant reference. A change can be a percentage change. so that a changed value is a1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% increase or decrease, including all integers and decimal points in between, relative to the relevant reference.

Internucleotidic linkage is used by persons having ordinary skill in the biomedical art to refer to the phosphorus-containing linkage between nucleotide units of an oligonucleotide and is interchangeable with inter-sugar linkage and phosphorus atom bridge. An internucleotidic linkage is a phosphodiester linkage, as found in naturally occurring DNA and RNA molecules. An internucleotidic linkage is a modified internucleotidic linkage wherein each oxygen atom of the phosphodiester linkage is optionally and independently replaced by an organic or inorganic moiety. Such an organic or inorganic moiety is selected from but not limited to ═S, ═Se, ═NR′, —SR′, —SeR′, —N(R′)2, B(R′)3, —S—, —Se—, and —N(R′)—, wherein each R′ is independently defined and described. An internucleotidic linkage is a phosphotriester linkage, phosphorothioate diester linkage, or modified phosphorothioate triester linkage. Persons of ordinary skill in the biomedical art know that the internucleotidic linkage can exist as an anion or cation at a given pH due to the existence of acid or base moieties in the linkage. An internucleotide linkage can be a chiral linkage.

Linked, when used regarding two or more moieties, is used by persons having ordinary skill in the biomedical art to mean that the moieties are physically associated or connected with one another to form a molecular structure that is sufficiently stable so that the moieties remain associated under the conditions in which the linkage is formed and under the conditions in which the new molecular structure is used, e.g., physiological conditions. The linkage can be a covalent linkage. The linkage can be noncovalent. Moieties can be linked either directly or indirectly. When two moieties are directly linked, they are covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. When two moieties are called being linked by a linker or linking moiety or linking portion, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which can be protected depending upon the conditions), and in sufficient amount, to produce a reasonable yield.

Long-term administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

Modulator agent is used by persons having ordinary skill in the biomedical art to refer to an agent, condition, or event whose presence, level, degree, type, or form correlates with increased, decreased, or otherwise altered level or activity of another agent, i.e., the modulated agent or the target agent. A modulator agent can be or include an agent of any chemical class including, small molecules, polypeptides, nucleic acids, carbohydrates, lipids, metals, or any other entity that shows the relevant activating activity. A modulator agent can be direct (in which case it exerts its influence directly upon its target); a modulator agent can be indirect (in which case it exerts its influence by other than binding to its target; e.g., by interacting with a regulator of the target, so that level or activity of the target is altered. A modulator is a binding agent that is a protein, e.g., an antibody or a nucleic acid, e.g., an antisense oligonucleotide that binds a target, e.g., a protein or nucleic acid so that level, form, or activity of the target is altered. The altered level, form or activity is an increased or decreased level of altered mRNA, protein expressed from the target nucleic acid sequence. Persons having ordinary skill in the biomedical art know that a modulator agent can bind to and potentially modulate a binding target, which binding causes an increase or decrease in level or activity of a further modulated target. A modulator agent that binds to a nucleic acid target can alter level or activity of that target. A modulator agent can modulate an activity of that nucleic acid target, e.g., by altering its modification, splicing, 5′ cap formation, or 3′ end formation, transport, or translation, etc., so that a level of a desired product, e.g., mRNA, is increased or decreased or can modulate a downstream target, such as a polypeptide encoded by such nucleic acid target. A modulator agent can be or comprise an oligonucleotide that binds to a primary transcript and alters its splicing pattern so that level or activity of a particular spliced form, e.g., mature mRNA is increased or decreased or otherwise altered, which can, in turn achieved increased or decreased level of a product, e.g., a polypeptide that is or is encoded by such particular spliced form.

Modulator therapy is used by persons having ordinary skill in the biomedical art to refer to administration of a modulator agent that modulate a particular target of interest to achieve a desired therapeutic effect. Modulator therapy involves administering a single dose of a modulator. Modulator therapy involves administering multiple doses of a modulator. Modulator therapy involves administering a modulator according to a dosing regimen known or expected to achieve the therapeutic effect, because such result was established to a designated degree of statistical confidence, e.g., through administration to a relevant population. Modulator therapy involves delivery of modulator agent as described in this specification. A modulator agent can be or comprise a binding agent that is a protein, e.g., an antibody or a nucleic acid, e.g., an antisense oligonucleotide that binds a target, e.g., a protein or nucleic acid, so that level, form, or activity of the target is altered. A modulator agent can bind to and potentially modulate a binding target, which binding causes an increase or decrease in level or activity of a further modulator target. A modulator agent that binds to a nucleic acid target can alter level or activity of that target. A modulator agent can modulate an activity of that nucleic acid target, e.g., by increasing or decreasing its modification, splicing, 5′ cap formation, or 3′ end formation, transport, or translation, etc., so that a level of a desired product, e.g., mRNA, is generated or can modulate a downstream target, such as a polypeptide encoded by such nucleic acid target. A modulator agent can be or comprise an oligonucleotide that binds to a primary transcript and alters its splicing pattern so that level or activity of a particular spliced form, e.g., mature mRNA is generated, which can, in turn achieved increased or decreased level of a product, e.g., a polypeptide that is or is encoded by such particular spliced form.

Moiety is used by persons having ordinary skill in the biomedical art to refer to a defined chemical group or entity with a particular structure or activity.

MuSK is known to persons having ordinary skill in the biomedical art as a receptor tyrosine kinase comprised extracellularly of three Ig and one CRD/Fz domain and an intracellular tyrosine domain (TK). The best understood function of MuSK is at the neuromuscular junction (NMJ) where agrin-LRP4 binding to the Ig1 domain triggers MuSK TK activity and synapse differentiation (Kim et al., 2008; Zhang et al., 2008a). MuSK is also a BMP co-receptor that binds BMP and its receptors ALK3 and 6, upregulates BMP signaling and shapes the composition of the transcriptional response in myogenic cells. See Yilmaz et al. (2016). This BMP signaling pathway neither regulates nor requires MuSK TK activity nor is it activated by agrin-LRP4. The MuSK Ig3 domain is necessary for high affinity BMP binding but is dispensable for agrin-LRP4 TK activation. The Ig3 domain is endogenously alternatively spliced, including in the brain. See Garcia-Osta et al., 2006; Hesser et al., 1999. Because BMP signaling induces NSC quiescence and can inhibit integration of newborn neurons, restraining BMP drive by reducing MuSK-BMP signaling can increase neurogenesis.

MuSK-BMP pathway is known to persons having ordinary skill in the biomedical art. The brain harbors neural stem cells (NSCs) that generate neurons and glial cells throughout life. Moreno-Jimenez et al. (2019); Steiner et al. (2019). BMPs regulate at least two important neural stem cell decision points: (1) quiescence, where proliferating stem cells exit the cell cycle and return to replenish a reserve pool that can supply fresh stem cells; and; (2) differentiation into mature progeny. See Mira et al. (2010). Manipulating the BMP pathway in neural stem cells is an attractive target for regulating neurogenesis in the adult brain.

Nanoparticle is used by persons having ordinary skill in the biomedical art to refer to a particle having a diameter of less than 1000 nanometers (nm). A nanoparticle has a diameter of less than 300 nm, as defined by the National Science Foundation. A nanoparticle has a diameter of less than 100 nm as defined by the United States National Institutes of Health. nanoparticles are micelles because they comprise an enclosed compartment, separated from the bulk solution by a micellar membrane, typically comprised of amphiphilic entities which surround and enclose a space or compartment, e.g., to define a lumen. A micellar membrane comprises at least one polymer, such as a biocompatible or biodegradable polymer.

Nucleic acid, in its broadest sense, is used by persons having ordinary skill in the biomedical art to refer to any compound or substance that is or can be incorporated into an oligonucleotide chain. A nucleic acid is a compound or substance that is or can be incorporated into an oligonucleotide chain via a phosphodiester linkage. Nucleic acid can refer to an individual nucleic acid residue, e.g., a nucleotide or nucleoside. Nucleic acid can refer to an oligonucleotide chain comprising individual nucleic acid residues. A nucleic acid is or comprises RNA; a nucleic acid is or comprises DNA. A nucleic acid is, comprises, or consists of one or more natural nucleic acid residues. A nucleic acid is, comprises, or consists of one or more nucleic acid analogs. A nucleic acid analog differs from a nucleic acid because it does not utilize a phosphodiester backbone. A nucleic acid is, comprises, or consists of one or more peptide nucleic acids, which are known in the biomedical art and have peptide bonds instead of phosphodiester bonds in the backbone, are considered within the scope of the invention. A nucleic acid can have one or more phosphorothioate or 5′-N-phosphoramidite linkages rather than phosphodiester bonds. A nucleic acid is, comprises, or consists of one or more natural nucleosides, e.g., adenosine, thymidine, guanosine, cytidine, uridine, deoxyadenosine, deoxythymidine, deoxy guanosine, and deoxycytidine. A nucleic acid is, comprises, or consists of one or more nucleoside analogs, e.g., 2-aminoadenosine, 2-thiothymidine, inosine, pyrrolo-pyrimidine, 3 methyl adenosine, 5-methylcytidine, C5 propynyl-cytidine, C5 propynyl-uridine, 2 aminoadenosine, C5-bromouridine, C5-fluorouridine, C5-iodouridine, C5-propynyl-uridine, C5 propynyl-cytidine, C5-methylcytidine, 2-aminoadenosine, 7-deazaadenosine, 7 deazaguanosine, 8-oxoadenosine, 8-oxoguanosine, (6)-methylguanine, 2-thiocytidine, methylated bases, intercalated bases, and combinations thereof. A nucleic acid comprises one or more modified sugars, e.g., 2′-fluororibose, ribose, 2′ deoxyribose, arabinose, and hexose as compared with those in natural nucleic acids. A nucleic acid has a nucleotide sequence that encodes a functional gene product such as an RNA or protein. A nucleic acid includes one or more introns. nucleic acids are prepared by one or more of isolation from a natural source, enzymatic synthesis by polymerization based on a complementary template (in vivo or in vitro), reproduction in a recombinant cell or system, and chemical synthesis. A nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. A nucleic acid is partly or wholly single stranded; a nucleic acid is partly or wholly double stranded. A nucleic acid can have a nucleotide sequence comprising at least one element that encodes, or is the complement of a sequence that encodes, a polypeptide. A nucleic acid has enzymatic activity.

Prodrug is used by persons having ordinary skill in the biomedical art to refer to an entity that, when administered to an organism, is metabolized in the body to deliver an active, e.g., therapeutic or diagnostic agent of interest. Typically, such metabolism involves removal of at least one prodrug moiety so that the active agent is formed. Various forms of prodrugs are known by persons having ordinary skill in the biomedical art. As with other compounds described in this specification, prodrugs can be provided in any of a variety of forms, e.g., crystal forms, salt forms etc. prodrugs are provided as pharmaceutically acceptable salts thereof.

Operably linked is used by persons having ordinary skill in the biomedical art to refer to a juxtaposition wherein the components described are in a relationship permitting them to function in their intended manner. A control element operably linked to a functional element is associated so expression or activity of the functional element is achieved under conditions compatible with the control element. operably linked control elements, e.g., promoters, enhancers, etc. are contiguous, e.g., covalently linked with the coding elements of interest; control elements act in trans- or cis- with the coding functional element of interest.

Or shall have the inclusive meaning of the term, unless the context clearly indicates the disjunctive meaning of the term

Patient is used by persons having ordinary skill in the biomedical art to refer to any organism to which a provided composition, e.g., an agonizing agent such as an ASO is or can be administered, e.g., for experimental, diagnostic, prophylactic, cosmetic, or therapeutic purposes. Typical patients include animals, e.g., mammals such as mice, rats, rabbits, non-human primates, or humans. A patient is a human. A patient is suffering from or susceptible to one or more disorders or conditions. A patient displays one or more symptoms of a disorder or condition. A patient was diagnosed with one or more disorders or conditions. the disorder or condition is Alzheimer's disease (AD) or other disease characterized by neurodegeneration. the patient is receiving or has received certain therapy to diagnose or to treat a disease, disorder, or condition.

Pharmaceutical composition is used by persons having ordinary skill in the biomedical art to refer to an active agent, e.g., an agonizing agent, formulated together with one or more pharmaceutically acceptable carriers. Active agent is present in unit dose amount appropriate for administration in a therapeutic regimen that shows a statistically significant probability of achieving a predetermined therapeutic effect when administered to a relevant population. pharmaceutical compositions can be specially formulated for administration in solid or liquid form, including those adapted for oral administration, drenches, aqueous or non-aqueous solutions or suspensions, tablets, e.g., those targeted for buccal, sublingual, and systemic absorption, boluses, powders, granules, pastes for application to the tongue. parenteral administration, e.g., by subcutaneous, intramuscular, intravenous, intraperitoneal, intrathecal, intravenous, intraventricular or epidural injection as a sterile solution or suspension, or sustained-release formulation; topical application as a cream, ointment, or a controlled-release patch or spray applied to the skin, lungs, or oral cavity; intravaginally or intrarectally, e.g., as a pessary, cream, or foam; sublingually; ocularly; transdermally; or nasally, pulmonary, and to other mucosal surfaces.

Pharmaceutically acceptable is used by persons having ordinary skill in the biomedical art to refer to those compounds, materials, compositions or dosage forms which are, within the scope of sound medical judgment, suitable for contact with the tissues of humans and animals without excessive toxicity, irritation, allergic response, or other problem or complication, commensurate with a reasonable benefit/risk ratio.

Pharmaceutically-acceptable carrier is used by persons having ordinary skill in the biomedical art to mean a pharmaceutically-acceptable material, composition or vehicle, such as a liquid or solid filler, diluent, excipient, or solvent encapsulating material, involved in carrying or transporting the compound from one organ, or portion of the body, to another organ, or portion of the body. Each carrier must be acceptable in the sense of being compatible with the other ingredients of the formulation and not injurious to the patient. Pharmaceutically-acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates or polyanhydrides; and other non-toxic compatible substances employed in pharmaceutical formulations.

Pharmaceutically acceptable salt is used by persons having ordinary skill in the biomedical art to refer to salts of such compounds that are appropriate for use in pharmaceutical contexts, i.e., salts which are, within the scope of sound medical judgment, suitable for contact with the tissues of humans and lower animals without undue toxicity, irritation, allergic response and the like, and are commensurate with a reasonable benefit/risk ratio. Pharmaceutically acceptable salts are well known in the biomedical art. Berge et al. (1977) describes pharmaceutically-acceptable salts in detail. Pharmaceutically acceptable salt include nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the biomedical art such as ion exchange. Pharmaceutically-acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. A provided compound comprises one or more acidic groups, e.g., an oligonucleotide, and a pharmaceutically-acceptable salt is an alkali, alkaline earth metal, or ammonium, e.g., an ammonium salt of N(R)3, wherein each R is independently defined and described in this disclosure salt. Representative alkali or alkaline earth metal salts include sodium, lithium, potassium, calcium, magnesium, and the like. A pharmaceutically-acceptable salt is a sodium salt. A pharmaceutically-acceptable salt is a potassium salt. A pharmaceutically-acceptable salt is a calcium salt. pharmaceutically-acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. A provided compound comprises more than one acid groups. An oligonucleotide can comprise two or more acidic groups, e.g., in natural phosphate linkages or modified internucleotidic linkages. A pharmaceutically-acceptable salt, or generally a salt, of such a compound comprises two or more cations, which can be the same or different. in a pharmaceutically-acceptable salt (or generally, a salt), all ionizable hydrogen, e.g., in an aqueous solution with a pKa no more than about 11, 10, 9, 8, 7, 6, 5, 4, 3, or 2; no more than about 7; no more than about 6; no more than about 5; no more than about 4; no more than about 3 in the acidic groups are replaced with cations. each internucleotidic linkage, e.g., phosphate group, independently exists in its salt form, e.g., if sodium salt, —O—P(O)(ONa)—O—). A pharmaceutically-acceptable salt is a sodium salt of an oligonucleotide. A pharmaceutically-acceptable salt is a sodium salt of an oligonucleotide, wherein each acidic phosphate and modified phosphate group exists as a salt form (all sodium salt).

Polypeptide, which is interchangeably used in this specification with the term protein, is used by persons having ordinary skill in the biomedical art to refer to a polymer of at least three amino acid residues. A polypeptide comprises one or more, or all, natural amino acids. A polypeptide comprises one or more, or all non-natural amino acids. A polypeptide comprises one or more, or all, D-amino acids. A polypeptide comprises one or more, or all, L-amino acids. A polypeptide comprises one or more pendant groups or other modifications, e.g., modifying or attached to one or more amino acid side chains, at the polypeptide's N terminus, at the polypeptide's C-terminus, or any combination thereof. A polypeptide comprises one or more modifications such as acetylation, amidation, aminoethylation, biotinylation, carbamylation, carbonylation, citrullination, deamidation, deimination, eliminylation, glycosylation, lipidation, methylation, pegylation, phosphorylation, sumoylation, or combinations thereof. A polypeptide can participate in one or more intra- or inter-molecular disulfide bonds. A polypeptide can be cyclic or can comprise a cyclic portion. A polypeptide is not cyclic or comprises no cyclic portion. A polypeptide is linear. A polypeptide can comprise a stapled polypeptide. A polypeptide participates in non-covalent complex formation by non-covalent or covalent association with one or more other polypeptides, e.g., as in an antibody. A polypeptide has an amino acid sequence that occurs in nature. A polypeptide has an amino acid sequence that does not occur in nature. A polypeptide has an amino acid sequence that is engineered because it is designed or produced through action of the hand of man. The term polypeptide can be appended to a name of a reference polypeptide, activity, or structure and can refer to polypeptides that share the relevant activity or structure and thus can be considered members of the same class or family of polypeptides. For each such class, this specification provides or persons having ordinary skill in the biomedical art know exemplary polypeptides within the class whose amino acid sequences or functions are known; such exemplary polypeptides are reference polypeptides for the polypeptide class or family. A member of a polypeptide class or family shows significant sequence homology or identity with, shares a common sequence motif, e.g., a characteristic sequence element with, or shares a common activity, optionally at a comparable level or within a designated range with a reference polypeptide of the class or with all polypeptides within the class. A member polypeptide shows an overall sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more or includes at least one region, e.g., a conserved region that can comprise a characteristic sequence element that shows high sequence identity, often greater than 90% or even 95%, 96%, 97%, 98%, or 99%. Such a conserved region usually encompasses at least 3-4 and often up to 20 or more amino acids; a conserved region encompasses at least one stretch of at least 2, 3, 4, 5, 6, 7, 8, 9, 10, 11, 12, 13, 14, 15 or more contiguous amino acids. A useful polypeptide can comprise a fragment of a parent polypeptide. A useful polypeptide as can comprise a plurality of fragments, each of which is found in the same parent polypeptide in a different spatial arrangement relative to one another than is found in the polypeptide of interest, e.g., fragments that are directly linked in the parent can be spatially separated in the polypeptide of interest or vice versa, or fragments can be present in a different order in the polypeptide of interest than in the parent, so the polypeptide of interest is a derivative of its parent polypeptide.

Prevent or prevention, when used in connection with the occurrence of a disease, disorder, or condition, are used by persons having ordinary skill in the biomedical art to refer to reducing the risk of developing the disease, disorder or condition or to delaying onset of one or more characteristics or symptoms of the disease, disorder or condition. Prevention can be considered complete when onset of a disease, disorder or condition was delayed for a predefined period of time.

Recombinant is used by persons having ordinary skill in the biomedical art to refer to polypeptides that are designed, engineered, prepared, expressed, created, manufactured, or isolated by recombinant means, such as polypeptides expressed using a recombinant expression vector transfected into a host cell; polypeptides isolated from a recombinant, combinatorial human polypeptide library; polypeptides isolated from an animal, e.g., a mouse, rabbit, sheep, fish, etc. that is transgenic or otherwise was manipulated to express a gene or genes, or gene components that encode or direct expression of the polypeptide or one or more components, portions, elements, or domains thereof; or polypeptides prepared, expressed, created or isolated by any other means that involves splicing or ligating selected nucleic acid sequence elements to one another, chemically synthesizing selected sequence elements, or otherwise generating a nucleic acid that encodes or directs expression of the polypeptide or one or more components, portions, elements, or domains thereof. one or more of such selected sequence elements is found in nature. one or more of such selected sequence elements is designed in silico. one or more such selected sequence elements results from mutagenesis, e.g., in vivo or in vitro of a known sequence element, e.g., from a natural or synthetic source such as in the germline of a source organism of interest, e.g., of a human, a mouse, etc.

Small molecule is used by persons having ordinary skill in the biomedical art to mean a low molecular weight organic or inorganic compound. A small molecule is less than about 5 kilodaltons (kDa) in size. A small molecule is less than about 4 kDa, 3 kDa, about 2 kDa, or about 1 kDa. the small molecule is less than about 800 daltons (Da), about 600 Da, about 500 Da, about 400 Da, about 300 Da, about 200 Da, or about 100 Da. A small molecule is less than about 2000 g/mol, less than about 1500 g/mol, less than about 1000 g/mol, less than about 800 g/mol, or less than about 500 g/mol. A small molecule is not a polymer. A small molecule does not include a polymeric moiety. A small molecule is not or does not comprise a protein or polypeptide, e.g., is not an oligopeptide or peptide. A small molecule is not or does not comprise a polynucleotide, e.g., is not an oligonucleotide. A small molecule is not or does not comprise a polysaccharide. A small molecule is not a glycoprotein, proteoglycan, glycolipid, etc. A small molecule is not a lipid. A small molecule is a modulating agent, e.g., is an inhibiting agent or an activating agent. A small molecule is biologically active. A small molecule is detectable, e.g., comprises at least one detectable moiety. A small molecule is a therapeutic agent. Persons having ordinary skill in the biomedical art know that small molecule compounds described in this specification can be provided or used in any of a variety of forms such as crystal forms, salt forms, protected forms, pro-drug forms, ester forms, isomeric forms, e.g., optical or structural isomers, isotopic forms, etc. Persons having ordinary skill in the biomedical art know that some small molecule compounds have structures that can exist in one or more stereoisomeric forms. Such a small molecule can be used under this disclosure in the form of an individual enantiomer, diastereomer or geometric isomer, or can be in the form of a mixture of stereoisomers; such a small molecule can be used under this disclosure in a racemic mixture form. Persons having ordinary skill in the biomedical art know that some small molecule compounds have structures that can exist in one or more tautomeric forms. such a small molecule can be used under this disclosure in the form of an individual tautomer, or in a form that interconverts between tautomeric forms. Persons having ordinary skill in the biomedical art know that some small molecule compounds have structures that permit isotopic substitution. such a small molecule can be used in accordance with this disclosure in one or more isotopically modified forms, or mixtures thereof. reference to a particular small molecule compound can relate to a specific form of that compound. A particular small molecule compound can be provided or used in a salt form, e.g., in an acid-addition or base-addition salt form, depending on the compound. The salt form can be a pharmaceutically-acceptable salt form. When a small molecule compound exists or is found in nature, that compound can be provided or used under this specification in a form different from that in which it exists or is found in nature. Persons having ordinary skill in the biomedical art know that preparing a particular small molecule compound that contains an absolute or relative amount of the compound, or of a particular form thereof, that differs from the absolute or relative regarding another component of the preparation, optionally including another form of the compound amount of the compound or form present in a reference preparation of interest, e.g., in a primary sample from a source of interest such as a biological or environmental source is distinct from the compound as it exists in the reference preparation or source. A preparation of a single stereoisomer of a small molecule compound can be considered a different form of the compound than a racemic mixture of the compound. A particular salt of a small molecule compound can be considered a different form from another salt form of the compound; a preparation that contains only a form of the compound that contains one conformational isomer ((Z) or (E)) of a double bond can be considered a different form of the compound from one that contains the other conformational isomer ((E) or (Z)) of the double bond; a preparation in which one or more atoms is a different isotope than is present in a reference preparation can be considered a different form; etc.

Specific binding is used by persons having ordinary skill in the biomedical art to refer to an ability to discriminate between possible binding partners in the environment in which binding is to occur. A binding agent that interacts with one particular target when other potential targets are present is said to bind specifically to the target, e.g., a target amino acid or nucleic acid sequence on a target protein/gene of interest with which it interacts. specific binding is assessed by detecting or determining association between the binding agent and its partner; specific binding is assessed by detecting or determining dissociation of a binding agent-partner complex; specific binding is assessed by detecting or determining ability of the binding agent to compete an alternative interaction between its partner and another entity. specific binding is assessed by performing such detections or determinations across a range of concentrations.

Specificity is used by persons having ordinary skill in the biomedical art to refer to a measure of the ability of a particular ligand to distinguish its binding partner from other potential binding partners.

Subject is used by persons having ordinary skill in the biomedical art to refer to an organism, typically a mammal, e.g., a human, also including prenatal human forms. A subject is suffering from a relevant disease, disorder or condition, e.g., Alzheimer's disease (AD) or other disease characterized by neurodegeneration. A subject is susceptible to a disease, disorder, or condition. A subject displays one or more symptoms or characteristics of a disease, disorder, or condition. A subject need not display any symptom or characteristic of a disease, disorder, or condition. A subject can be someone with one or more features characteristic of susceptibility to or risk of a disease, disorder, or condition. A subject can be a patient.

Substantially is used by persons having ordinary skill in the biomedical art to refer to the qualitative condition of exhibiting total or near-total extent or degree of a characteristic or property of interest. Persons having of ordinary skill in the biological arts know that biological and chemical phenomena rarely go to completion or proceed to completeness or achieve or avoid an absolute result. The term substantially is therefore used in this specification to capture the potential lack of completeness inherent in many biological and chemical phenomena.

Substantial identity is used by persons having ordinary skill in the biomedical art to refer to a comparison between amino acid or nucleic acid sequences. Two sequences are generally considered in the biomedical to be substantially identical if they contain identical residues in corresponding positions. As is well known in this art, amino acid or nucleic acid sequences can be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al. (eds.), Bioinformatics Methods and Protocols (Methods in Molecular Biology, Vol. 132), Humana Press, 1999. In addition to identifying identical sequences, these programs indicate identity. two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. the relevant stretch is a complete sequence. the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suffering from is used by persons having ordinary skill in the biomedical art to refer to an individual with a disease, disorder, or condition, e.g., Alzheimer's disease (AD) or other disease characterized by neurodegeneration was diagnosed with or displays one or more symptoms of a disease, disorder, or condition.

Susceptible to is used by persons having ordinary skill in the biomedical art to refer to an individual who is susceptible to a disease, disorder, or condition, e.g., Alzheimer's disease (AD) or other disease characterized by neurodegeneration is one who has a higher risk of developing the disease, disorder, or condition than does a member of the general public. An individual who is susceptible to a disease, disorder or condition may not have been diagnosed with the disease, disorder, or condition. An individual who is susceptible to a disease, disorder, or condition can exhibit symptoms of the disease, disorder, or condition. An individual who is susceptible to a disease, disorder, or condition may not exhibit symptoms of the disease, disorder, or condition. An individual who is susceptible to a disease, disorder, or condition will develop the disease, disorder, or condition. An individual who is susceptible to a disease, disorder, or condition will not develop the disease, disorder, or condition.

Symptoms are reduced is used by persons having ordinary skill in the biomedical art to refer to when one or more symptoms of a particular disease, disorder or condition, e.g., Alzheimer's disease (AD) or other disease characterized by neurodegeneration is reduced in magnitude, e.g., intensity, severity, etc. or frequency. For purposes of clarity, a delay in the onset of a particular symptom is considered one form of reducing the frequency of that symptom.

Target gene is used by persons having ordinary skill in the biomedical art to refer to a gene whose expression is to be modulated, e.g., through modifying splice activity, e.g., by inducing exon-skipping. The term target portion or target region is used by persons having ordinary skill in the biomedical art to refer to a contiguous portion of the nucleotide sequence of a target gene. A target portion or target region is one or more exons within the target gene sequence. A target portion can be from about 8-36 nucleotides in length, e.g., about 10-20 or about 15-30 nucleotides in length. A target portion length can have specific value or subrange within the afore-mentioned ranges. A target portion can be between about 15-29, 15-28, 15 27, 15-26, 15-25, 15-24, 15-23, 15-22, 15-21, 15-20, 15-19, 15-18, 15-17, 18-30, 18-29, 18 28, 18-27, 18-26, 18-25, 18-24, 18-23, 18-22, 18-21, 18-20, 19-30, 19-29, 19-28, 19-27, 19 26, 19-25, 19-24, 19-23, 19-22, 19-21, 19-20, 20-30, 20-29, 20-28, 20-27, 20-26, 20-25, 20 24, 20-23, 20-22, 20-21, 21-30, 21-29, 21-28, 21-27, 21-26, 21-25, 21-24, 21-23, or 21-22 nucleotides in length.

Therapeutic agent is used by persons having ordinary skill in the biomedical art to refer to any agent that, when administered to a subject, has a therapeutic effect or elicits a desired biological or pharmacological effect. A therapeutic agent is any substance that can alleviate, ameliorate, relieve, inhibit, prevent, delay onset of, reduce severity of, or reduce incidence of one or more symptoms or features of a disease, disorder, or condition, e.g., one or more symptoms or features of Alzheimer's disease (AD) or other disease characterized by neurodegeneration.

Therapeutically effective amount is used by persons having ordinary skill in the biomedical art to mean an amount of a substance, e.g., a therapeutic agent, composition, or formulation that elicits a desired biological response when administered as part of a therapeutic dosing regimen. A therapeutically effective amount of a substance is an amount that is sufficient, when administered to a subject suffering from or susceptible to a disease, disorder, or condition, to treat, diagnose, prevent, or delay the onset of the disease, disorder, or condition. Persons having ordinary skill in this art know that the effective amount of a substance can vary depending on such factors as the desired biological endpoint, the substance to be delivered, the target cell or tissue, etc. The effective amount of compound in a formulation to treat a disease, disorder, or condition is the amount that alleviates, ameliorates, relieves, inhibits, prevents, delays onset of, reduces severity of or reduces incidence of one or more symptoms or features of the disease, disorder, or condition, e.g., one or more symptoms or features of Alzheimer's disease (AD) or another disease characterized by neurodegeneration. A therapeutically effective amount is administered in a single dose. Several unit doses are required to deliver a therapeutically effective amount.

Treating is used by persons having ordinary skill in the biomedical art to refer to providing treatment, i.e., providing any medical or surgical management of a subject. The treatment can be provided to reverse, alleviate, inhibit the progression of, prevent or reduce the likelihood of a disease, disorder, or condition, or to reverse, alleviate, inhibit or prevent the progression of, prevent or reduce the likelihood of one or more symptoms or manifestations of a disease, disorder or condition. Treating can include administering an agent to the subject following the development of one or more symptoms or manifestations indicative of Alzheimer's disease (AD) or another other disease characterized by neurodegeneration, e.g., to reverse, alleviate, reduce the severity of, or inhibit or prevent the progression of the condition or to reverse, alleviate, reduce the severity of, or inhibit or one or more symptoms or manifestations of the condition. A composition of the disclosure can be administered to a subject who has developed Alzheimer's disease (AD) or another other disease characterized by neurodegeneration or is at increased risk of developing such a disorder relative to a member of the general population. A composition of the disclosure can be administered prophylactically, i.e., before development of any symptom or manifestation of the condition. The subject will typically be at risk of developing the condition.

Variant as used in the context of biomolecules, e.g., nucleic acids, e.g., antisense oligonucleotides (ASOs), proteins, or small molecules, refers to a molecule that shows significant structural identity with a reference molecule but differs structurally from the reference molecule, e.g., in the presence or absence or in the level of one or more chemical moieties as compared to the reference entity. A variant also differs functionally from its reference molecule. Whether a particular molecule is properly considered to be a variant of a reference molecule is based on its degree of structural identity with the reference molecule. Any biological or chemical reference molecule has characteristic structural elements. A variant is a distinct molecule that shares one or more such characteristic structural elements but differs in at least one aspect from the reference molecule. To give but a few examples, a polypeptide can have a characteristic sequence element comprised of a plurality of amino acids having designated positions relative to one another in linear or three-dimensional space or contributing to a particular structural motif or biological function; a nucleic acid can have a characteristic sequence element comprised of a plurality of nucleotide residues having designated positions relative to on another in linear or three-dimensional space. A variant polypeptide or nucleic acid can differ from a reference polypeptide or nucleic acid because of one or more differences in amino acid or nucleotide sequence or one or more differences in chemical moieties, e.g., carbohydrates, lipids, phosphate groups that are covalently components of the polypeptide or nucleic acid, e.g., that are attached to the polypeptide or nucleic acid backbone. A variant polypeptide or nucleic acid shows an overall sequence identity with a reference polypeptide or nucleic acid at least 85%, 86%, 87%, 88%, 89%, 90%, 91%, 92%, 93%, 94%, 95%, 96%, 97%, or 99%. A variant polypeptide or nucleic acid does not share at least one characteristic sequence element with a reference polypeptide or nucleic acid. A reference polypeptide or nucleic acid has one or more biological activities. A variant polypeptide or nucleic acid shares one or more of the biological activities of the reference polypeptide or nucleic acid. A variant polypeptide or nucleic acid lacks one or more of the biological activities of the reference polypeptide or nucleic acid. A variant polypeptide or nucleic acid shows a reduced level of one or more biological activities as compared to the reference polypeptide or nucleic acid. A polypeptide or nucleic acid of interest is considered a variant of a reference polypeptide or nucleic acid if it has an amino acid or nucleotide sequence identical to that of the reference but for a few sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. A variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a small number, e.g., fewer than about 5, about 4, about 3, about 2, or about 1 number of substituted, inserted, or deleted, functional residues (i.e., residues that participate in a particular biological activity relative to the reference. A variant polypeptide or nucleic acid comprises not more than about 5, about 4, about 3, about 2, or about 1 addition or deletion, and comprises no additions or deletions, as compared to the reference. A variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. A reference polypeptide or nucleic acid is found in nature. A reference polypeptide or nucleic acid is a human polypeptide or nucleic acid.

Vector is used by persons having ordinary skill in the biomedical art to refer to a nucleic acid molecule capable of transporting another nucleic acid to which it was linked. One type of vector is a plasmid, which refers to a circular double stranded DNA loop into which additional DNA segments can be ligated. Another type of vector is a viral vector, wherein additional DNA segments can be ligated into the viral genome. Some vectors autonomously replicate in a host cell into which they are introduced, e.g., bacterial vectors having a bacterial origin of replication and episomal mammalian vectors. Other vectors, e.g., non-episomal mammalian vectors can be integrated into the genome of a host cell upon introduction into the host cell and are replicated along with the host genome. Certain vectors can direct the expression of genes to which they are operatively linked. Such vectors are known in the biomedical art as expression vectors. Standard techniques can be used for recombinant DNA, oligonucleotide synthesis, and tissue culture and transformation, e.g., electroporation, lipofection. Enzymatic reactions and purification techniques can be performed according to manufacturer's specifications or as commonly accomplished in the biomedical art. The foregoing techniques and procedures can be generally performed according to conventional methods well-known in the biomedical art and as described in various general and more specific references cited and discussed throughout this specification. See, e.g., Molecular Cloning: A Laboratory Manual (2012).

Unless otherwise defined herein, scientific and technical terms used with this application shall have the meanings that are commonly understood by persons having ordinary skill in the biomedical art. This invention is not limited to the particular methodology, protocols, and reagents, etc., described herein and as such can vary.

The disclosure described herein does not concern a process for cloning humans, processes for modifying the germ line genetic identity of humans, uses of human embryos for industrial or commercial purposes or processes for modifying the genetic identity of animals likely to cause them suffering with no substantial medical benefit to man or animal, and also animals resulting from such processes.

### MuSK Ig2 Modulator Agents.

In some embodiments, the present disclosure provides technologies for modulating biological events mediated by a MuSK Ig2 domain. For example, in some embodiments, the present disclosure provides technologies for disrupting or otherwise interfering with one or more binding interactions between a MuSK Ig2 domain and a binding partner (e.g., a BMP Receptor or an autoantibody), as described herein.

In some embodiments, a MuSK Ig2 modulator agent increases level or activity of one or more MuSK polypeptides (e.g., ΔIg2-MuSK) that lacks an effective Ig2 domain, e.g., because such domain has been mutated, removed, or otherwise inactivated (e.g., by blocking, modification, etc.). Alternatively or additionally, in some embodiments, a MuSK Ig2 modulator agent blocks, inactivates, mutates, or removes a functional Ig2 domain from MuSK, or achieves, supports, or contributes to such blocking, inactivation, mutation or removal. Thus, in some embodiments, a MuSK Ig2 modulator agent may be considered a ΔIg2-MuSK agonizing agent.

In some embodiments, the present disclosure provides technologies that relate to MuSK Ig2 modulator agents—including, for example, providing such agents themselves, or providing methods or reagents for identifying, characterizing, manufacturing or using them or compositions that comprise or deliver them.

In some embodiments, a MuSK Ig2 modulator agent may interact directly with a MuSK polypeptide (e.g., with full-length MuSK or with ΔIg2-MuSK). In some embodiments, a MuSK Ig2 modulator agent may not interact directly with a MuSK polypeptide but rather, through some other interaction (e.g., with a precursor or regulator or downstream product of MuSK), impacts level or activity of ΔIg2-MuSK.

In principle, an MuSK Ig2 modulator agent may be of any chemical class (e.g., small molecule, polypeptide [e.g., antibody], nucleic acid, etc.).

In some embodiments, a MuSK Ig2 modulator agent is agent that downregulates the MuSK Ig2 domain protein expression, the MuSK Ig2 domain gene expression, or the MuSK Ig2 activation of BMP signaling through one or more of the BMPRs. Without wishing to be bound by any particular theory, the present disclosure proposes that such a MuSK Ig2 modulator agent may thereby induce neurogenesis or may reduce muscle atrophy or stimulate muscle growth (e.g., myogenesis).

In some embodiments, a MuSK Ig2 modulator agent is an agonizing agent that increases expression of MuSK ΔIg2.

In some particular embodiments, described in more detail herein, a MuSK Ig2 modulator agent may be or comprise a small molecule.

In some particular embodiments, described in more detail herein, a MuSK Ig2 modulator agent may be or comprise an antibody that binds to a MuSK polypeptide (e.g., an antibody that blocks MuSK Ig2 or that sequesters one or more MuSK polypeptide forms that include functional Ig2). Alternatively or additionally, in some particular embodiments, a MuSK Ig2 modulator agent may be or comprise an agent that disrupts (e.g., interferes with, competes, etc.) an interaction between MuSK Ig2 and an antibody (e.g., an autoantibody); for example, in some embodiments, a MuSK Ig2 modulator agent may be or comprise the MuSK Ig2 domain, while lacking one or more other MuSK elements (e.g., in some embodiments, a MuSK Ig2 modulator agent may be or comprise a soluble peptide that is or comprises the MuSK Ig2 domain or an autoantibody-binding-portion thereof.

In some particular embodiments, described in more detail herein, a MuSK Ig2 modulator agent may be or comprise a nucleic acid agent. For example, in some embodiments, a MuSK Ig2 modulator agent may be or comprise a nucleic acid (e.g., a gene therapy vector or an RNA therapeutic such as an mRNA) encoding a MuSK form (e.g., ΔIg2-MuSK) that lacks a functional Ig2 domain. Alternatively or additionally, in some embodiments, a nucleic acid MuSK Ig2 modulator agent may be or comprise an oligonucleotide, such as a MuSK Ig2-targeted exon-skipping oligonucleotide, a MuSK Ig2-targeted CRISPR/Cas9 gRNA (e.g., that modifies or removes Ig2), a MuSK Ig2-targeted siRNA (e.g., that inhibits production/expression of MuSK Ig2, for example from a transcript that encodes it), or a MuSK Ig2-targeted shRNA.

### Small Molecules.

In some embodiments, a MuSK Ig2 modulator agent may be or comprise a small molecule compound.

In some embodiments, a small molecule MuSK Ig2 modulator agent targets therapeutic agent that targets MuSK splicing; for example, in some embodiments such a small molecule compound enhances splicing event(s) that generate(s) message encoding ΔI2-MuSK or inhibits splicing event(s) that generate(s) message(s) encoding other MuSK splice variants. In some embodiments, a small molecule MuSK Ig2 modulator agent alters the BMP signaling pathway. In some embodiments, a small molecule MuSK Ig2 modulator agent alters the BMP signaling pathway through targeting a BMP receptor, which further induces neurogenesis or may reduce muscle atrophy or stimulate muscle growth (e.g., myogenesis).

In some embodiments, a small molecule MuSK Ig2 modulator agent targets one or more of type I BMP receptors, ALK3 (ALK is Anaplastic lymphoma kinase) and ALK6, and type I activin receptor ALK4. In some embodiments, a small molecule MuSK Ig2 modulator agent is an ALK inhibitor. In some embodiments, a small molecule MuSK Ig2 modulator agent is an ALK inhibitor selected from the group consisting of crizotinib, ceritinib, alectinib, brigatinib, lorlatinib.

In some embodiments, a small molecule MuSK Ig2 modulator agent targets the MuSK Ig2 domain or BMP so that level or activity of a MuSK/BMP complex is reduced. In some such embodiments, a small molecule MuSK Ig2 modulator agent inhibits formation of or disrupts such complex(es). In some embodiments, such a MuSK Ig2 modulator agent competes with a BMP receptor for binding to MuSK Ig2 or competes with MuSK Ig2 for binding to a BMP receptor.

### Antibody Agents.

In some embodiments, a MuSK Ig2 modulator agent is an antibody agent.

In some embodiments, such an antibody agent specifically binds to a MuSK polypeptide. In some embodiments, an antibody agent targeting MuSK specifically binds to the Ig2 domain of a MuSK polypeptide.

In some embodiments, an antibody targeting the Ig2 domain of MuSK protein may bind specifically to the Ig2 domain relative to the Ig1 or Ig3 domains of MuSK.

In some embodiments, an antibody MuSK Ig2 modulator agent targets the MuSK Ig2 domain or a BMP receptor so that level or activity of a MuSK/BMP receptor complex is reduced. In some such embodiments, an antibody MuSK Ig2 modulator agent inhibits formation of or disrupts such complex(es). In some embodiments, such an antibody MuSK Ig2 modulator agent competes with a BMP receptor for binding to MuSK Ig2 or competes with MuSK Ig2 for binding to BMP receptor.

In some embodiments, an anti-MUSK antibody agent is internalized by a cell (e.g., a cell with a neuronal cell type). In some embodiments, an antibody or antigen-binding fragment thereof described herein can be or comprise an immunoglobulin, heavy chain antibody, light chain antibody, or other protein scaffold with antibody-like properties, and other immunological binding moiety known in the art, including a Fab fragment, a Fab′ fragment, a F(ab′)2 fragment, a Fv fragment, a disulfide-bonded Fv fragment, a scFv fragment, a diabody, a triabody, a tetrabody, a minibody, a maxibody, a tandab, BiTe, and any combination thereof. In some embodiments, anti-MUSK antibodies or antigen-binding fragments thereof target, for example, the Ig2 domain of MuSK. In some embodiments, such antibodies, or antigen-binding fragments thereof, may inhibit or substantially prevent the binding of a BMP receptor to the MuSK Ig2 domain.

An antibody can be an immunoglobulin molecule of four polypeptide chains, e.g., two heavy (H) chains and two light (L) chains. A heavy chain can include a heavy chain variable domain and a heavy chain constant domain. A heavy chain constant domain can include CH1, hinge, CH2, CH3, and sometimes CH4 regions. A light chain can include a light chain variable domain and a light chain constant domain. A light chain constant domain can include a CL. A heavy chain variable domain of a heavy chain and a light chain variable domain of a light chain can typically be further subdivided into regions of variability, termed complementarity determining regions (CDRs), interspersed with regions that are more conserved, termed framework regions (FR). Such heavy chain and light chain variable domains can each include three CDRs and four framework regions, arranged from amino-terminus to carboxyl-terminus in the following order: FR1, CDR1, FR2, CDR2, FR3, CDR3, FR4, one or more of which can be engineered as described herein.

In some embodiments, an antibody agent (e.g., anti-MUSK antibodies) can include various heavy chains and light chains described herein. In some embodiments, an antibody can include two heavy chains and light chains. In various embodiments, the present disclosure encompasses an antibody including at least one heavy chain or light chain as disclosed herein, at least one heavy chain or light chain framework domain as disclosed herein, at least one heavy chain or light chain CDR domain as disclosed herein, or any heavy chain or light chain constant domain as disclosed herein.

In some embodiments, an antibody agent is or comprises a monoclonal antibody. Typically, monoclonal antibodies are obtained from a population of substantially homogeneous antibodies, i.e., the individual antibodies comprising the population are substantially identical except for possible naturally occurring mutations that may be present in minor amounts. Thus, the modifier monoclonal as used herein. indicates the character of the antibody as not being a mixture of discrete antibodies. In some embodiments, monoclonal antibodies directed to a particular epitope are derived from a single cell line (e.g., a B cell line).

In some embodiments, an antibody agent (e.g., an anti-MuSK antibody) may be or comprise a polyclonal antibody. In contrast to monoclonal antibodies, polyclonal antibodies typically represent a population of heterogeneous antibodies, i.e., the antibodies in a particular population include structural variation, for example, affinity for different epitope(s) on a particular antigen (e.g., the Ig2 domain of MuSK, or a region within the Ig2 domain). Several methods of producing polyclonal antibodies are known in the art, including use of multiple subcutaneous or intraperitoneal injections of the relevant antigen into an animal, optionally including co-administration of one or more adjuvants.

### Oligonucleotides.

In some embodiments, a MuSK Ig2 modulator agent as described herein is or comprises an oligonucleotide.

Synthetic oligonucleotides provide useful molecular tools in a wide variety of applications. For example, oligonucleotides are useful in therapeutic, diagnostic, research, and new nanomaterials applications. Using naturally occurring nucleic acids (e.g., unmodified DNA or RNA) is limited, for example, by their susceptibility to endonucleases and exonucleases. Various synthetic counterparts were developed to circumvent these shortcomings. These include synthetic oligonucleotides that contain chemical modification, e.g., base modifications, sugar modifications, backbone modifications, etc., which render these molecules less susceptible to degradation and improve other properties of oligonucleotides. Chemical modifications may also lead to certain undesired effects, such as increased toxicities, etc.

The present disclosure encompasses the recognition that structural elements of oligonucleotides, such as base sequence, chemical modifications (e.g., modifications of sugar, base, or internucleotidic linkages, and patterns thereof), or stereochemistry (e.g., stereochemistry of backbone chiral centers (chiral internucleotidic linkages), or patterns thereof), can have significant impact on properties, e.g., stability, splicing-altering capabilities, etc. In some embodiments, oligonucleotide properties can be adjusted by optimizing chemical modifications (modifications of base, sugar, or internucleotidic linkage) or stereochemistry (pattern of backbone chiral centers).

In some embodiments, the present disclosure demonstrates that oligonucleotide compositions comprising oligonucleotides with controlled structural elements, e.g., controlled chemical modification, provide unexpected properties, including but not limited to those described herein. In some embodiments, provided compositions comprising oligonucleotides having chemical modifications (e.g., base modifications, sugar modification, internucleotidic linkage modifications, etc.) have improved properties, such as improved splicing-altering capabilities, or improved protein binding profile, or improved delivery, etc. Particularly, in some embodiments, the present disclosure provides compositions and methods for altering splicing of transcripts. In some embodiments, the present disclosure provides compositions and methods for improving splicing of transcripts. In some embodiments, altered transcript splicing by provided compositions and methods include production of products having desired or improved biological functions, or knockdown of undesired product by, e.g., modifying splicing products so that undesired biological functions can be suppressed or removed.

In some embodiments, a splicing product is mRNA. In some embodiments, alteration comprises skipping one or more exons. In some embodiments, splicing of a transcript is improved in that exon skipping increases levels of mRNA and proteins that have improved beneficial activities compared with absence of exon skipping.

In some embodiments, splicing of a transcript is improved in that exon skipping lowers levels of mRNA and proteins that have undesired activities compared with absence of exon skipping. In some embodiments, a target is knocked down through exon skipping which, by skipping one or more exons, causes premature stop codon or frameshift mutations.

In some embodiments, an oligonucleotide of the disclosure includes one or more natural nucleobase or one or more modified nucleobases derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products).

Modified nucleobases also include expanded-size nucleobases in which one or more aryl rings, such as phenyl rings, were added.

In some embodiments, modified nucleobases are of any one of the following structures, optionally substituted:

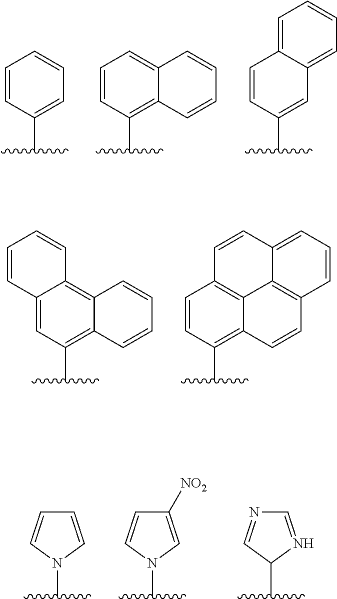

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a universal base that is not a nucleobase in the most classical sense, but that functions similarly to a nucleobase. One representative example of such a universal base is 3-nitropyrrole.

In some embodiments, an oligonucleotide described herein includes nucleosides that incorporate modified nucleobases or nucleobases covalently bound to modified sugars. Some examples of nucleosides that incorporate modified nucleobases include 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; I-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-methylcytosine, 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, nucleosides include 6′-modified bicyclic nucleoside analogs that have either (R) or (S)-chirality at the 6′-position and include the analogs described in U.S. Pat. No. 7,399,845. In other embodiments, nucleosides include 5′-modified bicyclic nucleoside analogs that have either (R) or (S)-chirality at the 5′-position and include the analogs described in U.S. Publ. No. 20070287831. In some embodiments, a nucleobase or modified nucleobase is 5-bromouracil, 5-iodouracil, or 2,6-diaminopurine. In some embodiments, a nucleobase or modified nucleobase is modified by substitution with a fluorescent moiety.

In some embodiments, an oligonucleotide described herein includes one or more modified nucleotides wherein a phosphate group or linkage phosphorus in the nucleotides are linked to various positions of a sugar or modified sugar. As non-limiting examples, the phosphate group or linkage phosphorus can be linked to the 2′, 3′, 4′ or 5′ hydroxyl moiety of a sugar or modified sugar. Nucleotides that incorporate modified nucleobases as described herein are also contemplated in this context.

Other modified sugars can also be incorporated within an oligonucleotide molecule. In some embodiments, a modified sugar contains one or more substituents at the 2′ position including one of the following: —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. Examples of substituents include, and are not limited to, —O(CH₂)ₙOCH₃, and —O(CH₂)ₙNH₂, wherein n is from 1 to about 10, MOE, DMAOE, DMAEOE.

In some embodiments, the 2′-OH of a ribose is replaced with a substituent including one of the following: —H, —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe (MOE).

Modified sugars also include locked nucleic acids (LNAs). In some embodiments, the locked nucleic acid has the structure indicated below. A locked nucleic acid of the structure below is indicated, wherein Ba represents a nucleobase or modified nucleobase as described herein, and wherein R^{2s} is —OCH₂C4′-

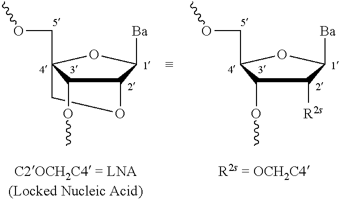

In some embodiments, the present invention provides an oligonucleotide comprising one or more modified internucleotidic linkages independently having the structure of formula I:

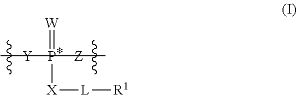

wherein:
P* is an asymmetric phosphorus atom and is either Rp or Sp;
W is O, S or Se;
each of X, Y and Z is independently —O—, —S—, —N(-L-R¹)—, or L;
L is a covalent bond or an optionally substituted, linear or branched C₁-C₁₀ alkylene, wherein one or more methylene units of L are optionally and independently replaced by an optionally substituted C₁-C₆ alkylene, C₁-C₆ alkenylene,

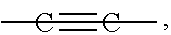

—C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
R¹ is halogen, R, or an optionally substituted C₁-C₅₀ aliphatic wherein one or more methylene units are optionally and independently replaced by an optionally substituted C₁-C₆ alkylene, C₁-C₆ alkenylene,

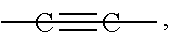

—C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
each R′ is independently —R, —C(O)R, —CO₂R, or —SO₂R, or:
   two R′ on the same nitrogen are taken together with their intervening atoms to form an optionally substituted heterocyclic or heteroaryl ring, or
   two R′ on the same carbon are taken together with their intervening atoms to form an optionally substituted aryl, carbocyclic, heterocyclic, or heteroaryl ring;
-Cy- is an optionally substituted bivalent ring selected from phenylene, carbocyclylene, arylene, heteroarylene, or heterocyclylene;
each R is independently hydrogen, or an optionally substituted group selected from C₁-C₅ aliphatic, phenyl, carbocyclyl, aryl, heteroaryl, or heterocyclyl; and
each [symbol] independently represents a connection to a nucleoside.

In some embodiments, the internucleotidic linkage having the structure of formula I is

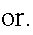

Among other things, the present disclosure provides oligonucleotides of various designs, which may comprise various nucleobases and patterns thereof, sugars and patterns thereof, internucleotidic linkages and patterns thereof, or additional chemical moieties and patterns thereof as described in the present disclosure. In some embodiments, provided oligonucleotides can downregulates the MuSK Ig2 domain protein expression, the MuSK Ig2 domain gene expression, or the MuSK Ig2 activation of BMP signaling level through a BMP receptor, thereby increasing adult hippocampal neurogenesis (AHN) and improving cognition in AD or achieving (e.g., inducing, enhancing, etc.) muscle regeneration and growth. In some embodiments, provided oligonucleotides can direct a decrease in the expression, level or activity of MuSK Ig2 domain or one or more of its products in a cell of a subject or patient. In some embodiments, a cell normally expresses or produces protein encoded by MuSK Ig2 domain. In some embodiments, provided oligonucleotides can direct a decrease in the expression, level or activity of MuSK Ig2 domain gene or a gene product and has a base sequence which consists of, comprises, or comprises a portion (e.g., a span of 10, 11, 12, 13, 14, 15, 16, 17, 18, 19, 20 or more contiguous bases) of the base sequence of a oligonucleotide disclosed herein, wherein each T can be independently substituted with U and vice versa, and the oligonucleotide comprises at least one non-naturally-occurring modification of a base, sugar or internucleotidic linkage.

As described herein, the highly abundant full-length MuSK harbors the BMP Receptor (ALK)-binding Ig2 domain and potentiates BMP signaling and thus restrains neurogenesis and muscle regeneration/growth (e.g., myogenesis). In contrast, ΔIg2-MuSK has lower BMP signaling through the BMP receptor (ALK) and promotes AHN and improves cognition. In some embodiments, the present disclosure provides exon-skipping ASOs that switch MuSK from the AHN-restraining full length MuSK to the AHN-permissive ΔIg2-MuSK splice form.

In some embodiments, one or more skipped exons are selected from exon 5a and 5b or MuSK gene. In some embodiments, exon 5a of MuSK is skipped. In some embodiments, exon 5b of MuSK is skipped. In some embodiments, both exons 5a and 5b of MuSK are skipped.

In various embodiments, an active compound is an oligonucleotide that directs skipping of one or more exons in a MuSK gene. In various embodiments, an active compound is an oligonucleotide that directs skipping of multiple exons in a MuSK gene. In some embodiments, an active compound is an oligonucleotide that directs skipping of exon 5a, exon 5b, or both in a MuSK gene. In some embodiments, an active compound is an oligonucleotide that directs skipping of exon 5a in a MuSK gene. In some embodiments, an active compound is an oligonucleotide that directs skipping of exon 5b in a MuSK gene. In some embodiments, an active compound is an oligonucleotide that directs skipping of exons 5a and 5b in a MuSK gene. In some embodiments, a plurality of oligonucleotides may be used together. In some such embodiments, two or more different exon skipping oligonucleotides (e.g., at least one that directs skipping of exon 5a and one that directs skipping of exon 5b) may be used in combination. Alternatively or additionally, in some embodiments, at least one exon skipping oligonucleotide may be used in combination with at least one degrading oligonucleotide (e.g., that targets a transcript for RNase H degradation) which, for example, may target MuSK transcript(s) that include a functional Ig2 domain, or portion thereof.

In some embodiments, oligonucleotides are provided or utilized in salt forms. In some embodiments, oligonucleotides are provided as salts comprising negatively-charged internucleotidic linkages (e.g., phosphorothioate internucleotidic linkages, natural phosphate linkages, etc.) existing as their salt forms. In some embodiments, oligonucleotides are provided as pharmaceutically acceptable salts. In some embodiments, oligonucleotides are provided as metal salts. In some embodiments, oligonucleotides are provided as sodium salts. In some embodiments, oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

### Model Systems

Among other things, the present disclosure provides model systems useful as described herein. For example, in some embodiments, provided model system(s) that can be used to screen, validate, characterize, assess, or identify one or more MuSK MR agonizing agents.

In some embodiments, a model system provided herein is or comprises an artificially engineered cell line. In some embodiments, an engineered cell line is an immortalized MuSK−/− myogenic cell line.

In some embodiments, a model system provided herein is or comprises an engineered mouse as described herein. In some embodiments, a provided mouse is or comprises an ΔIg2-MuSK mouse.

In some embodiments, provided model systems (e.g., cell lines or mice) are used to screen, validate, characterize, assess, or identify agents as described herein, including, for example, small molecule agents, antibody agents, oligonucleotide agents, etc., and combinations thereof. In some embodiments, activity of such agents is compared to an appropriate reference (e.g., a positive or a negative control). In some embodiments, an appropriate reference may be or comprise absence of any agent, or presence of an agent of known activity or performance in the model system. In some embodiments, an appropriate reference may be a historical reference. In some embodiments, an appropriate reference may be a contemporary or simultaneous reference.

In some embodiments, a model system provided herein co-crystal structure of MuSK and BMP receptors. In some embodiments, the co-crystal structure can be used to guide design of small molecule MuSK MR agonizing agents through computer-based simulations. In some embodiments, the co-crystal structure can be used to guide high through-put screening of small molecule MuSK MR agonizing agents. In some embodiments, the small molecule MuSK MR agonizing agents target MuSK. In some embodiments, small molecule MuSK MR agonizing agents target one or more of type I BMP receptors, ALK3 (ALK is Anaplastic lymphoma kinase) and ALK6, and type I activin receptor ALK4, for example, an ALK inhibitor (e.g., crizotinib, ceritinib, alectinib, brigatinib, lorlatinib).

### Characterization of MuSK Ig2 Modulator Agents.

MuSK Ig2 modulator agents can be identified, assessed or characterized for one or more their physical/chemical properties or biological activities. Persons having ordinary skill in the biomedical art know a variety of approaches, including particular assays, that can be used for such identification, assessment, or characterization.

A small molecule MuSK Ig2 modulator agent might interfere with interaction between MuSK Ig2 and BMP receptor, for example by binding directly to MuSK Ig2 or to BMP receptors. Such agents can be characterized by direct binding assays, e.g., that assess their affinity for, specificity to, or one or more kinetic or thermodynamic features of their interaction with, their targets to MuSK Ig2 or to BMP receptor, or by competitive binding assays, e.g., that assess their ability to disrupt or undue pre-formed complexes of MuSK Ig2 and BMP receptor or to reduce complex formation. Such binding assays are desirably performed at several concentrations. Such binding assays can also be performed with full-length MuSK, or with some other polypeptide or agent that is or comprises MuSK Ig2.

A MuSK Ig2 modulator agent, e.g., an antibody or small molecule that binds the Ig2 domain of MuSK, when contacted with a cell expressing MuSK, will compete with BMP receptor for binding of the Ig2 domain. Such an antibody agent specifically binds to an epitope of MuSK expressed in a particular cell type, e.g., neuronal cell types. Such an antibody agent can have a binding affinity, e.g., as measured by a dissociation constant) for MuSK protein, e.g., the Ig2 domain of MuSK protein of at least about 10⁻⁴M, at least about 10⁻⁵M, at least about 10⁻⁶M, at least about 10⁻⁷M, at least about 10-$M, at least about 10⁻⁹M, or lower. Persons having ordinary skill in the biomedical art know that binding affinity, e.g., as measured by a dissociation constant) can be influenced by non-covalent intermolecular interactions such as hydrogen bonding, electrostatic interactions, hydrophobic and van der Waals forces between the two molecules. Binding affinity between a ligand and its target molecule can be affected by other molecules. Persons having ordinary skill in the biomedical art will be familiar with a variety of technologies for measuring binding affinity or dissociation constants under this disclosure, including, e.g., but not limited to ELISAs, gel-shift assays, pull-down assays, equilibrium dialysis, analytical ultracentrifugation, surface plasmon resonance (SPR), bio-layer interferometry, grating-coupled interferometry, and spectroscopic assays.

Competition assays can identify an antibody that competes with the anti-MuSK antibody agents described in this specification for binding to the Ig2 domain of MuSK. Such a competing antibody binds to the same epitope within the Ig2 domain of MuSK that is bound by the anti-MuSK antibodies described in this specification. Exemplary epitope mapping methods are known. See, e.g., Morris, Epitope mapping protocols, Methods in Molecular Biology, 66 (1996).

Assays can be provided for identifying anti-MuSK antibody agents thereof having biological activity. Assays can be provided for identifying anti-MuSK antibody agents thereof having neutralization activity for MuSK. Antibody agents having such biological activity in vivo or in vitro can be also provided. An antibody of the disclosure can be assayed for such biological activity.

The biological activity of an anti-MuSK antibody agent can refer to, for example, binding affinity for a particular MuSK epitope, e.g., within the Ig2 domain, neutralization or inhibition of MuSK binding to BMP, neutralization or inhibition of MuSK activity in vivo, e.g., IC₅₀, pharmacokinetics, and cross-reactivity e.g., with non-human homologs or orthologs of the MUSK protein, or with other proteins or tissues. Other biological properties or characteristics of an antigen-binding agent recognized in the biomedical art can include, for example, avidity, selectivity, solubility, folding, immunotoxicity, expression, and formulation. These properties or characteristics can be observed, measured, or assessed using standard techniques including, but not limited to, ELISA, competitive ELISA, surface plasmon resonance analysis (BIACORE™), or Kinetic Exclusion Assay (KINEXA™), in vitro or in vivo neutralization assays, receptor-ligand binding assays, cytokine or growth factor production or secretion assays, and signal transduction and immunohistochemistry assays.

A MuSK Ig2 modulator agent as described in this specification is characterized in that, for example, the MuSK Ig2 modulator agent, e.g., an agonizing oligonucleotide, when contacted with a cell expressing MuSK, will increase the level or activity of MuSK ΔIg2 mRNA or protein.

A MuSK Ig2 modulator oligonucleotide is characterized by its ability to alter splicing activity of MuSK pre-mRNA in a cell. For example, a cell can be transfected with a MuSK Ig2 modulator oligonucleotide, and after a period of incubation, expression of an alternative form of processed form of a MuSK RNA transcript, e.g., where exons 5a and 5b were skipped, can be measured by RT-PCR. For example, the efficiency of MuSK exon skipping in cultured cells greater than 10%, 20%, 30%, 40%, 50%, 60%, 70%, 80%, 90%, 95%, or 95%.

A MuSK Ig2 modulator oligonucleotide can increase MuSK ΔIg2 mRNA. A MuSK Ig2 modulator oligonucleotide can alter splicing of MuSK pre-mRNA. A MuSK Ig2 modulator oligonucleotide can promote the skipping or inclusion of exon 5a or exon 5b.

Modulation of expression of MuSK ΔIg2 can be measured in a bodily fluid of a subject treated with MuSK Ig2 modulator oligonucleotide, which might contain cells; tissue; or organ of the animal. Methods of obtaining samples for analysis, such as body fluids, e.g., sputum, serum, or CSF, tissues, e.g., biopsy, or organs, and methods of preparation of the samples to allow for analysis are well known to persons having ordinary skill in the biomedical art. The effects of treatment on a subject can be assessed by measuring biomarkers associated with the target gene expression in one or more biological fluids, tissues or organs, collected from an animal contacted with one or more compositions described in this application.

An increase in MuSK ΔIg2 mRNA means that the intracellular level of MuSK ΔIg2mRNA is higher than a reference level, such as the level of MuSK ΔIg2mRNA in a control (for example in a subject that is not being administered a MuSK Ig2 modulator oligonucleotide). An increase in intracellular MuSK ΔIg2 mRNA can be measured as an increase in the level of MuSK ΔIg2 protein or mRNA produced. An increase in MuSK ΔIg2 mRNA can be determined by, e.g., methods as described in this specification or by assay techniques such as RNA solution hybridization, nuclease protection, Northern hybridization, reverse transcription, gene expression monitoring with a microarray, antibody binding, enzyme linked immunosorbent assay (ELISA), nucleic acid sequencing, Western blotting, radioimmunoassay (RIA), other immunoassays, fluorescence activated cell analysis (FACS), or any other technique or combination of techniques that can detect MuSK ΔIg2 mRNA or protein, e.g., in a subject or a sample obtained from a subject.

By comparing the level of MuSK ΔIg2 mRNA in a sample obtained from a subject receiving a MuSK Ig2 modulator oligonucleotide treatment to a level of MuSK ΔIg2 mRNA in a subject not treated with a MuSK Ig2 modulator oligonucleotide, the extent to which the MuSK Ig2 modulator oligonucleotide treatment increased MuSK ΔIg2 mRNA can be determined. the reference level of MuSK ΔIg2 mRNA is obtained from the same subject before receiving MuSK Ig2 modulator oligonucleotide treatment. The reference level of MuSK ΔIg2 mRNA is a range determined by a population of subjects not receiving MuSK Ig2 modulator oligonucleotide treatment. The level of full-length MuSK mRNA is compared to the level of MuSK ΔIg2 mRNA. The ratio of the MuSK ΔIg2 mRNA to a full length MuSK mRNA, e.g., MuSK mRNA without exons 5a and 5b in a subject receiving a MuSK Ig2 modulator oligonucleotide treatment, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or higher than a reference ratio.

An increased level of MuSK ΔIg2 mRNA is, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or higher than a reference value.

The increase of MuSK ΔIg2 mRNA in a subject can be indicated by the increase of MuSK ΔIg2protein as compared to a reference level. the reference level of MuSK ΔIg2 protein is the MuSK ΔIg2protein level obtained from a subject having or at risk of having, e.g., Alzheimer's disease or a disease characterized by neurodegeneration, before treatment. Methods whereby bodily fluids, organs or tissues are contacted with an effective amount of one or more compositions described in this specification are also contemplated. Bodily fluids, organs or tissues can be contacted with one or more compositions comprising MuSK Ig2 modulator oligonucleotides, resulting in expression of MuSK ΔIg2 and modulation of MuSK expression in the cells of bodily fluids, organs or tissues. An effective amount of can be determined by monitoring the effect on functional MuSK ΔIg2protein expression of MuSK Ig2 modulator oligonucleotides that are administered to a subject or contacted to a cell.

A MuSK Ig2 modulator agent, when administered to a population of cells, e.g., comprising neural stem cells or neural progenitor cells (NPCs), increases the number of cells in an activated state, e.g., active proliferation. Cells within a population can be assessed for whether they are in an activated state by known methods in the biomedical art, including, e.g., an EdU assay, where EdU+ cycling cells are compared with total cell counts. A MuSK Ig2 modulator agent, when administered to a population of cells comprising neural stem cells, decreases the number of quiescent neural stem cells in the population or increases the number of activated neural stem cells.

A MuSK Ig2 modulator agent, when administered to a population of cells comprising neural stem cells or neural progenitor cells, increases the number of cells expressing genes associated with early neurons, e.g., Dex, or decreases the number of cells expressing genes associated with mature neurons, e.g., Map2, astrocytes, e.g., GFAP and S100b, or oligodendrocytes, e.g., CNPase and O4. A MuSK Ig2 modulator agent, when administered to a population of cells comprising neural stem cells or neural progenitor cells, increases the level of expression of genes associated with early neurons, e.g., Dex, or decreases the level of expression of genes associated with mature neurons, e.g., Map2, astrocytes, e.g., GFAP and S100b, or oligodendrocytes, e.g., CNPase and O4, in the population of cells.

A population of cells comprises neural stem cells that were induced to be neural stem cells, e.g., from stem cells such as embryonic stems cells or pluripotent stem cells.

A population of cells is obtained from a healthy subject. A population of cells is obtained from a subject suffering from a disease characterized by neurodegeneration or muscle disorder.

A MuSK Ig2 modulator agent, when contacted with a population of cells from a subject, increases neurogenesis or muscle regeneration or growth in a subject. A MuSK Ig2 modulator agent is contacted with the population of cells in vivo, for example, by injection into a subject. A MuSK Ig2 modulator agent is contacted with the population of cells ex vivo by obtaining a population of cells from a subject, and neurogenesis or muscle regeneration or growth is increased when the treated cells are re-introduced into the subject.

A MuSK Ig2 modulator agent, when administered to a subject, will increase neurogenesis, improve cognition, or achieve muscle regeneration or growth. Examples of methods to assess these biological effects are detailed in this specification.

### Production of Agonizing Antibodies.

Antibodies and antigen-binding fragments can be prepared or purified by any technique known in the biomedical art, which allows for the subsequent formation of a stable antibody or antibody fragment.

A nucleic acid encoding an anti-MuSK antibody agent of this disclosure can be easily isolated and sequenced by conventional procedures.

An expressed antibody of this disclosure can be uniformly purified after being isolated from a host cell. Isolation or purification of an antibody of this disclosure can be performed by a conventional method for isolating and purifying a protein. For example, not wishing to be bound by theory, a MuSK antibody agent of this disclosure can be recovered and purified from recombinant cell cultures by well-known methods including, but not limited to, protein. A purification, protein G purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (HPLC) can also be employed for purification. An antibody of this disclosure can be isolated or purified by additionally combining filtration, superfiltration, salting out, dialysis, etc.

Purified anti-MuSK agents of this disclosure can be characterized by ELISA, ELISPOT, flow cytometry, immunocytology, BIACORE™ analysis, SAPIDYNE KINEXA™ kinetic exclusion assay, SDS-PAGE and Western blot, or by HPLC analysis and by several other functional assays disclosed in this specification.

### Production of Agonizing Oligonucleotides.

An agonizing agent, e.g., an agonizing oligonucleotide described in this specification can be synthesized by standard methods known in the biomedical art, e.g., by use of an automated synthesizer. After chemical synthesis, e.g., solid-phase synthesis using phosphoramidite method) agonizing oligonucleotide molecules can be deprotected, annealed to ds molecules, and purified, e.g., by gel electrophoresis or HPLC. Protocols for preparation of agonizing oligonucleotides are known in the biomedical art.

An agonizing oligonucleotides can also be formed within a cell by transcription of RNA from an expression construct introduced into the cell. See, e.g., Yu et al., Proc. Natl. Acad. Sci. U.S.A., 99, 6047-6052 (2002). An expression construct for in vivo production of agonizing oligonucleotide molecules can include one or more antisense encoding sequences operably linked to elements necessary for the proper transcription of the antisense encoding sequences, including, e.g., promoter elements and transcription termination signals. Preferred promoters for such expression constructs include the polymerase-Ill HI-RNA promoter (see, e.g., Brummelkamp et al., Science 2002; 296:550-553) and the U6 polymerase-Ill promoter (see, e.g., Sui et al., Proc. Natl. Acad. Sci. USA 2002; Paul et al., Nature Biotechnol., 20, 505-508 (2002); and Yu et al., Proc. Natl. Acad. Sci. U.S.A., 99, 6047-6052) (2002). An agonizing oligonucleotide expression construct can further comprise one or more vector sequences that facilitate the cloning of the expression construct. Standard vectors that can be used include, e.g., pSilencer 2.0-U6 vector (Ambion Inc., Austin, TX, USA).

### Pharmaceutical Compositions.

This present disclosure provides pharmaceutical compositions that comprise or deliver agonizing agents as described in this specification. This disclosure also provides pharmaceutical compositions that are or comprise cell populations that were exposed to agonizing agents as described in this specification.

For example, a provided pharmaceutical composition can comprise or deliver a MuSK Ig2 modulator agent such as an antibody agent or nucleic acid agent that, when administered, achieves an increase in level or activity of a MuSK polypeptide, e.g., a MuSK ΔIg2polypeptide, or another MuSK variant polypeptide with disrupted Ig2 that lacks an Ig2domain functional for interaction with BMP. A provided pharmaceutical composition can comprise or deliver a population of cells exposed to a MuSK Ig2 modulator agent, so that neuronal cell number or activity is increased in the population.

A pharmaceutical composition can be or can comprise an active agent, e.g., an agonizing agent as described in this specification or a precursor thereof, combined with one or more pharmaceutically acceptable excipients. Persons having ordinary skill in the biomedical art know that components of a particular pharmaceutical composition can be influenced by route of administration of the pharmaceutical composition.

The compositions of the disclosure can be formulated for a variety of modes of administration, including systemic and topical or localized administration. Techniques and formulations generally can be found in Remington, The Science and Practice of Pharmacy (20th ed. 2000).

Compositions of this invention can be prepared and administered in several oral, parenteral, and topical dosage forms. The compositions of this invention can be administered by injection, e.g., intravenously, intramuscularly, intracutaneously, subcutaneously, intraduodenally, or intraperitoneally. The compositions described in this specification can be administered by inhalation, for example, intranasally. The composition of this invention can be administered transdermally. Several routes of administration, e.g., intramuscular, oral, transdermal can be used to administer the compositions of the invention.

A pharmaceutical composition as described in this specification can be formulated for delivery by a route selected from intravenous injection, intrathecal administration, oral administration, buccal administration, inhalation, nasal administration, topical administration, ophthalmic administration or otic administration. A pharmaceutical composition can be formulated for delivery by intrathecal administration. A pharmaceutical composition can be formulated for delivery by intravenous administration. A pharmaceutical composition can be formulated for delivery by oral administration.

Oligonucleotides and compositions can be delivered to the central nervous system. Oligonucleotides and compositions are delivered to the cerebrospinal fluid. In certain embodiments, oligonucleotides and compositions are administered to the brain parenchyma. Oligonucleotides and compositions can be delivered to an animal/subject by intrathecal administration, or intracerebroventricular administration. Broad distribution of oligonucleotides and compositions, described in this specification, within the central nervous system can be achieved with intraparenchymal administration, intrathecal administration, or intracerebroventricular administration.

Parenteral administration can be by injection, by, e.g., a syringe, a pump, etc. The injection can be a bolus injection. The injection can be administered directly to a tissue, such as striatum, caudate, cortex, hippocampus and cerebellum.

Methods of specifically localizing a pharmaceutical agent, such as by bolus injection, can decrease median effective concentration (EC₅₀) by a factor of 20, 25, 30, 35, 40, 45 or 50. The pharmaceutical agent in an antisense compound as further described in this specification. The targeted tissue can be brain tissue. The targeted tissue can be hippocampus tissue. Decreasing EC₅₀ can be desirable because it reduces the dose required to achieve a pharmacological result in a patient in need thereof.

An antisense oligonucleotide can be delivered by injection or infusion once every month, every two months, every ninety days, every three months, every six months, twice a year or once a year.

These pharmaceutical compositions can also contain suitable pharmaceutically-acceptable carriers comprising excipients and auxiliaries which facilitate processing of an active compound into preparations which can be used pharmaceutically. The preparations formulated for oral administration can be in tablets, dragees, capsules, or solutions.

Pharmaceutical preparations for oral use can be obtained by combining an active compound with solid excipients, optionally grinding a resulting mixture, and processing the mixture of granules, after adding suitable auxiliaries, if desired, to obtain tablets or dragee cores. Suitable excipients are fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), or polyvinylpyrrolidone (PVP: povidone). Disintegrating agents can be added, such as the cross-linked polyvinylpyrrolidone, agar, or alginic acid or a salt thereof such as sodium alginate.

Dragee cores are provided with suitable coatings. Concentrated sugar solutions can be used, which can optionally contain gum arabic, talc, polyvinylpyrrolidone, carbopol gel, polyethylene glycol (PEG), or titanium dioxide, lacquer solutions, and suitable organic solvents or solvent mixtures. Dye-stuffs or pigments can be added to the tablets or dragee coatings for identification or to characterize different combinations of active compound doses.

Pharmaceutical preparations that can be used orally include push-fit capsules made of gelatin, and soft, sealed capsules made of gelatin, and a plasticizer, such as glycerol or sorbitol. The push-fit capsules can contain the active ingredients in admixture with filler such as lactose, binders such as starches, or lubricants such as talc or magnesium stearate and, optionally, stabilizers. In soft capsules, an active compound can be dissolved or suspended in suitable liquids, such as fatty oils, liquid paraffin, or liquid polyethylene glycols (PEGs). Stabilizers can be added.

The pharmaceutical composition can be a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

Depending on the specific conditions being treated, pharmaceutical composition of this disclosure can be formulated into liquid or solid dosage forms and administered systemically or locally. The pharmaceutical composition can be delivered, for example, in a timed- or sustained-low release form as known to persons having ordinary skill in the biomedical art. Techniques for formulation and administration can be found in Remington, The Science and Practice of Pharmacy (20th ed. 2000). Suitable routes can include oral, buccal, by inhalation spray, sublingual, rectal, transdermal, vaginal, transmucosal, nasal or intestinal administration; parenteral delivery, including intramuscular, subcutaneous, intramedullary injections, and intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the pharmaceutical composition of the disclosure can be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are well-known in the biomedical art.

Use of pharmaceutically-acceptable inert carriers to formulate the compositions of this specification disclosed for the practice of the disclosure into dosages suitable for systemic administration is within the scope of the disclosure. With proper choice of carrier and suitable manufacturing practice, the compositions of this disclosure those formulated as solutions, can be administered parenterally, such as by intravenous injection.

Compositions as described in this specification can be formulated using pharmaceutically-acceptable carriers available in the biomedical art into dosages suitable for oral administration. Such carriers enable the compounds of the disclosure to be formulated as tablets, pills, capsules, liquids, gels, syrups, slurries, suspensions and the like, for oral ingestion by a subject, e.g., patient to be treated.

For nasal or inhalation delivery, one or more solubilizing, diluting, or dispersing substances such as, saline, preservatives, such as benzyl alcohol, absorption promoters, and fluorocarbons, can be employed.

A provided composition can comprise or deliver a precursor of an active agent, wherein the precursor becomes or releases active therapeutic agent upon administration. for example, a precursor can be or comprise a prodrug of a small molecule agonizing agent, or a nucleic acid that encodes a protein agonizing agent, etc.

A provided pharmaceutical composition can comprise or can deliver a therapeutically effective amount, e.g., an amount that is effective when administered according to an established protocol) of a provided oligonucleotide, which can be provided in a pharmaceutically-acceptable salt form, e.g., as a sodium salt, ammonium salt, etc. Such a provided pharmaceutical composition includes a relevant oligonucleotide and at least one pharmaceutically-acceptable inactive ingredient selected from pharmaceutically-acceptable diluents, pharmaceutically-acceptable excipients, and pharmaceutically-acceptable carriers. A salt form of a provided oligonucleotide comprises two or more cations, for example, up to the number of negatively charged acidic groups, e.g., phosphate, phosphorothioate, etc. in an oligonucleotide.

Pharmaceutically-acceptable salts are generally well known to persons having ordinary skill in the biomedical art, and can include, for example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically-acceptable salts can be found in, for example, Remington, The Science and Practice of Pharmacy (20th ed. 2000). Preferred pharmaceutically-acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

As known by a person having ordinary skill in the biomedical art, oligonucleotides can be formulated as several salts for, e.g., pharmaceutical uses. A salt is a metal cation salt or ammonium salt. A salt is a metal cation salt of an oligonucleotide. A salt is an ammonium salt of an oligonucleotide. Representative alkali or alkaline earth metal salts include sodium, lithium, potassium, calcium, magnesium, and the like. A salt is a sodium salt of an oligonucleotide. pharmaceutically-acceptable salts include, when appropriate, nontoxic ammonium, quaternary ammonium, and amine cations formed with counterions such as hydroxide, carboxylate, sulfate, phosphate, nitrate, sulfonate, phosphorothioate, etc. that can be within provided oligonucleotides. As appreciated by a person having ordinary skill in the biomedical art, a salt of an oligonucleotide can contain more than one cations, e.g., sodium ions, as there can be more than one anions within an oligonucleotide.

The provided oligonucleotides, and compositions thereof, can be effective over a wide dosage range. For example, in the treatment of adult humans, dosages from about 0.01 to about 1000 mg, from about 0.5 to about 100 mg, from about 1 to about 50 mg per day, and from about 5 to about 100 mg per day are examples of dosages that can be used. The exact dosage will depend upon the route of administration, the form in which the compound is administered, the subject to be treated, the body weight of the subject to be treated, and the preference and experience of the attending physician.

This disclosure provides technologies, e.g., compositions, methods, etc. for combination therapy, for example, with other therapeutic agents or medical procedures. Provided oligonucleotides or compositions can be used with one or more other therapeutic agents. Provided compositions comprise provided oligonucleotides and one or more other therapeutic agents. The one or more other therapeutic agents can have one or more different targets, or one or more different mechanisms toward targets, when compared to provided oligonucleotides in the composition. A therapeutic agent is an oligonucleotide. A therapeutic agent is a small molecule drug. A therapeutic agent is a protein. A therapeutic agent is an antibody. Several therapeutic agents can be used in accordance with this disclosure. Provided oligonucleotides or compositions thereof are administered before, concurrently with, or after one or more other therapeutic agents or medical procedures. Provided oligonucleotides or compositions thereof are administered concurrently with one or more other therapeutic agents or medical procedures. provided oligonucleotides or compositions thereof are administered prior to one or more other therapeutic agents or medical procedures. Provided oligonucleotides or compositions thereof are administered subsequent to one or more other therapeutic agents or medical procedures. Provided compositions comprise one or more other therapeutic agents.

### Production of Pharmaceutical Compositions.

For preparing pharmaceutical compositions from the compositions of the present disclosure, pharmaceutically acceptable carriers can be either solid or liquid. Solid form preparations include powders, tablets, pills, capsules, cachets, suppositories, and dispersible granules. A solid carrier can be one or more substance that can also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets can contain from 5% to 70% of the therapeutic agent. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term preparation includes the formulation of the active therapeutic agent with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously, as by stirring. The molten homogeneous mixture is then poured into convenient sized molds, allowed to cool, and to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for compositions of the invention are injectable, sterile solutions, in oily or aqueous solutions, and suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compositions of the invention can also be incorporated into liposomes or administered via transdermal pumps or patches. Pharmaceutical admixtures suitable for use in this invention include those described, for example, in Pharmaceutical Sciences 23^{rd} ed. (Elsevier, 2020).

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations intended to be converted, shortly before use, to liquid form preparations for oral administration. Such liquid forms include solutions, suspensions, and emulsions. These preparations can contain, besides the active component, colorants, flavors, stabilizers, buffers, artificial and natural sweeteners, dispersants, thickeners, solubilizing agents, and the like.

The pharmaceutical preparation can ne in unit dosage form. In such form the preparation is subdivided into unit doses containing appropriate quantities of the active component. The unit dosage form can be a packaged preparation, the package containing discrete quantities of preparation, such as packed tablets, capsules, and powders in vials or ampoules. The unit dosage form can be a capsule, tablet, cachet, or lozenge itself, or it can be the appropriate number of any of these in packaged form.

The quantity of active component in a unit dose preparation can be varied or adjusted according to the particular application and the potency of the active component. The composition can, if desired, also contain other compatible therapeutic agents.

### Administration

Those skilled in the art will appreciate that, in some embodiments, dosage administered to a subject, particularly a human, may vary, for example depending on the particular therapeutic or formulation employed, the method of administration, the dosing regimen, one or more characteristics of the particular subject being treated, etc. In some embodiments, a clinician skilled in the art will determine the therapeutically effective amount of a therapeutic to be administered to a human or other subject in order to treat or prevent a particular medical condition. The precise amount of the therapeutic required to be therapeutically effective will depend upon numerous factors, e.g., such as the specific activity of the therapeutic, and the route of administration, in addition to many subject-specific considerations, which are within those of skill in the art.

In some embodiments, administration may be ocular, oral, buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc.

Those skilled in the art, reading the present disclosure will appreciate that, in some embodiments, it may be desirable to achieve delivery of a MuSK Ig2 modulator agent to muscle. Alternatively, or additionally, in some embodiments, it may be desirable to achieve delivery of a MuSK Ig2 modulator agent to the CNS (e.g., the brain, such as the hippocampus or the subventricular region) or to the lung.

In some embodiments, an agent (e.g., an agonizing agent) is delivered via systemic delivery or local delivery to muscle (e.g., via intramuscular injection).

In some embodiments, a MuSK Ig2 modulator agent is administered using a viral vector to effectively deliver a MuSK Ig2 modulator agent in the form of a nucleic acid payload. In some embodiments, a viral vector targets certain cell types (e.g., myoblasts, myocytes, myotubes, satellite cells and myofibers). AAV1, AAV6, and AAV9 vectors were used to target different muscle cell types (See, for example, Arnett et al., Mol Ther Methods Clin Dev. 1. pii: 14038, 2014 and Riaz et al., Skeletal Muscle 5 (37) 2015).

Those skilled in the art, reading the present disclosure will appreciate that, in some embodiments, it may be desirable to achieve delivery of a MuSK Ig2 modulator agent to the CNS, and, in some embodiments to the brain. Those skilled in the art, reading the present disclosure will appreciate that, in some embodiments, it may be desirable to achieve delivery of a MuSK Ig2 modulator agent to muscle.

In some embodiments systemic administration achieves delivery to CNS (e.g., brain e.g., hippocampus or subventricular zone) or muscle. In some embodiments, an agent (e.g., an agonizing agent) is delivered to the central nervous system (CNS) or muscle, via intracerebroventricular administration.

Additionally, certain viral vectors are known to selectively target neurons, and to effectively deliver genetic payloads to the brain. For example, AAV2/1 vectors were established to effectively deliver nucleic acid payloads (e.g., gene therapy, encoded RNAs, etc.) to neuronal cells in the hippocampus. See, for example, Hammond et al., PLoS One, 12, e0188830 (2017); Guggenhuber et al., PLoS One, 5, e15707, (2010); Lawlor et al., Mol. Neurodeg. 2:11 (2007). Analogously, certain AAV vectors (e.g., AAV2/1 or AAV4 vectors) were established to target and effectively deliver nucleic acid payloads to certain cells in the subventricular zone cells. See, for example, Liu et al., Gene Therap. 12, 1503 (2005); Bockstael et al., Hum. Gene Therap. 23, 216 (2012).

For subjects suffering from or susceptible to a disease, disorder or condition associated with neurodegeneration, administration that achieves delivery to the CNS, e.g., to the brain (e.g., to the hippocampus or the subventricular region) may be desirable.

In some embodiments, effective delivery may be achieved by systemic administration of a composition as described herein. Alternatively, or additionally, in some embodiments, effective delivery may be achieved by local administration to the CNS, to the brain, or to the muscle, for example by intrathecal or intracavitary (e.g., intracerebroventricular) delivery.

Technologies for local administration to the CNS, to the brain, or to the muscle were developed and demonstrated to be effective, for example, for various protein therapeutics (see, for example Calias et al., Pharmacol. & Therap. 144:122, 2014), for small molecules (see, for example, Dodou Pharm 289:501, 2012), for cell compositions (see, for example, Eftekharzadeh et al., Iran J Basic Med Sci 18:520, 2015); and nucleic acid therapeutics (see, for example, Otsuka et al., J. Neurotrauma 28:1063, 2011; see also prescribing information for onasemnogene abeparvovec-xioi [sold under the brand name Zolgensma™] and that for nusinersen [sold under the brand name Spinraza™])

Those skilled in the art will be aware that intrathecal delivery may be particularly effective to achieve delivery to the hippocampus, including for cellular, protein, and nucleic acid therapeutics.

Systemic administration technologies (including, e.g., oral, parenteral, mucosal, etc.) are well established for a wide variety of agents. Systemic administration that achieves CNS, muscle, or brain delivery, in some embodiments, may depend on ability to cross the blood brain barrier (BBB).

Certain active agents or delivery systems are known to cross the BBB. Recent technologies were shown to achieve CNS or brain delivery even of agents, such as oligonucleotides, that had historically been considered to be particularly challenging in that regard. To give but one example, Min et al., Angew Chem. Int. Ed. Engl., (2020), incorporated herein by reference, describes glucose-coated polymeric nanocarriers that transport oligonucleotides across the BBB.

Incorporation of certain particular chemistries into oligonucleotide therapeutics can facilitate their travel across the BBB. For example, Khorkova et al., Nature Biotech 35:249, 2017, incorporated herein by reference) have described that: 2′-modified phosphorothioate oligonucleotides . . . may be particularly adaptable for CNS disorders, given their long half-life, with effects in the brain lasting up to 6 months following a single injection. In another type of sugar moiety modification, locked nucleic acids (LNAs), a bridge is introduced that connects the 2′ oxygen and 4′ carbon. This modification substantially elevates the melting temperature of the LNA-DNA and LNA-RNA hybrids, thus allowing the creation of shorter ODN-based compounds with increased bioavailability and reduced manufacturing costs. A recently proposed tricyclo-DNA, a conformationally constrained oligonucleotide analog, has three additional C-atoms between C(5′) and C(3′) of the sugar (FIG. 2). This modification increases stability, hydrophobicity and RNA affinity, and improves tissue uptake and BBB permeability.

For subjects suffering from or susceptible to a disease or disorder such as idiopathic pulmonary fibrosis (IPF), acute respiratory distress syndrome (ARDS), pneumonia, and lung complications due to viral infections, administration that achieves delivery to the lungs may be desirable.

### Oligonucleotides

In some embodiments, ASOs are developed to enhance their delivery to target site(s). As described in the art, the oligo load is covalently bound to a carrier or ligand, such as lipid particles, liposomes, nanoparticles, and more recently, the sugar N-acetyl galactosamine to enhance safer delivery to the target site. See Verma, Ann. Indian Acad. Neurol. 2018 21(1): 3-8.

Certain technologies were developed to improve the efficiency of cellular delivery of ASOs to target site, e.g., muscle. For example, aminoglycosides (AGs) are shown to improve the delivery of antisense phosphorodiamidate morpholino oligomer (PMO) both in vitro and in vivo. See Wang, et al., Mol. Ther. Nucleic Acids, 2019; 16: 663-674. Short cell-penetrating peptides (CPPs) that can be either directly attached to oligonucleotides through covalent linkages or through the formation of noncovalent nanoparticle complexes can facilitate cellular uptake. See McClorey et al.; Biomedicines, 6(2), 51 (2018). ASO fatty acid conjugates are also reported to enhance the functional uptake of antisense oligonucleotide (ASO) in the muscle. See Prakash et al.; Nucleic Acids Research, 47, 2019, 6029-6044.

Those skilled in the art will be familiar with eteplirsen (ExonDys 51), an approved treatment for Duchenne muscular dystrophy (DMD), which is a third-generation phosphorodiamidate morpholino ASO.

Eteplirsen, sold under the brand name Exondys 51™, (Sarepta Therapeutics’) causes exon 51 to be spliced out in pre-mRNA, restoring the reading frame in the 13% of patients with amenable frame-shifting mutations (See Crudele et al. Human Molecular Genetics, Volume 28, Issue R1, pp. R102-R107, 2019).

Eteplirsen is administered via intravenous infusion over 35 to 60 minutes. In particular, its recommended dosage is 30 mg/kg body weight weekly. In a single-dose vial, the pharmaceutical composition is formulated as a 100 mg/2 mL or 500 mg/mL (50 mg/mL) solution.

In some embodiments, an oligonucleotide therapeutic as described herein may be administered intravenously. In some such embodiments, such oligonucleotide therapeutic may be administered according to a regimen reasonably comparable to that used for eteplirsen [sold under the brand name Exondys 51™].

In some embodiments a lower dose of an agonizing oligonucleotide as described herein is 12 mg. In some embodiments, a total of 5 mg to 60 mg per dose of agonizing oligonucleotide is administered to a subject. In some embodiments, a total of 12 mg to 48 mg per dose of agonizing oligonucleotide is administered to a subject. In some aspects, a total of 12 mg to 36 mg per dose of agonizing oligonucleotide is administered to a subject. In some aspects, a total of 12 mg per dose of agonizing oligonucleotide is administered to a subject.

Those skilled in the art will be familiar with nusinersen [sold under the brand name Spinraza™], an antisense oligonucleotide therapeutic that targets the survival motor neuron-2 (SMN2)-directed gene transcript and is indicated for the treatment of spinal muscular atrophy (SMA) in pediatric and adult patients. Spinraza is administered intrathecally. In particular, its recommended dosage is 12 mg/5 mL (2.4 mg/mL) in a single-dose vial per administration, according to a regiment that involves four loading doses; the first three of which are administered at 14-day intervals, and the fourth of which is administered 30 days after the 3rd dose; a maintenance dose is administered once every 4 months thereafter. It is recommended that platelet count, coagulation laboratory testing, and quantitative spot urine protein testing is done at baseline, and prior to each dose.

In some embodiments, an oligonucleotide therapeutic as described herein may be administered intrathecally. In some such embodiments, such oligonucleotide therapeutic may be administered according to a regimen reasonably comparable to that used for nusinersen [sold under the brand name Spinraza™].

In some embodiments, an oligonucleotide therapeutic as described herein may be administered intrathecally. In some such embodiments, such oligonucleotide therapeutic may be administered according to a regimen reasonably comparable to that used for nusinersen [sold under the brand name Spinraza™].

### Cell Therapy

In light of the ability of MuSK Ig2 modulator agents, as described herein, to promote muscle regeneration (e.g., from cell populations that are or comprise SCs, MPCs, or myoblasts), those skilled in the art reading the present disclosure will appreciate that, among other things, the present disclosure provides technologies for enhancing level of neurons, SCs, MPCs, or myoblasts present in a cell population. That is, contacting an original cell population with a MuSK Ig2 modulator agent as described herein can generate a resulting population with an increased level of neurogenesis or muscle regeneration or growth as compared with that in the original population; administration of such MuSK Ig2 modulator agent as described herein can achieve such increase.

In some embodiments, an original cell population may be or comprise neurons, SCs, MPCs, or myoblasts. In some embodiments, an original cell population is or comprises embryonic stems cells or pluripotent stem cells. In some embodiments, embryonic stems cells or pluripotent stem cells are or were differentiated into neural or myogenic progenitor cells, for example using techniques known in the art (See e.g., Miyagoe-Suzuki et al., Stem Cells Int. 7824614 2017).

In some embodiments, as discussed above, such administration delivers the MuSK Ig2 modulator agent such that it is exposed to (i.e., contacts) a relevant original cell population in vivo (e.g., in a human, and in particular in an adult human, for example into muscle tissue, of such human).

In some embodiments, administration in accordance with the present disclosure contacts an MuSK Ig2 modulator agent with a population of cells (e.g., an original population of cells), that for example, may be or comprise neural stem cells (NCSs), SCs, MPCs, or myoblasts, ex vivo. For example, in some embodiments, a MuSK Ig2 modulator agent is administered ex vivo (e.g., in vitro) to a population of cells from a subject. In some embodiments, a population of cells obtained from a subject.

In some embodiments, a MuSK Ig2 modulator agent of particular use ex vivo may be or comprise a small molecule, and antibody, or a nucleic acid agent, or a combination thereof, In some particular such embodiments, one or more agents that is or comprises a nucleic acid (e.g., one or more gene therapy [e.g., nucleic acid vector or transcript], oligonucleotide, or gRNAs) may be particular useful for ex vivo or in vitro administration to cells. CRISPR/Cas modification of cell populations is an established and growing field, and those skilled in the art will appreciate applicability of such strategies in accordance with the present disclosure, e.g., to modify or disrupt MuSK Ig2 domain sequences. Alternatively or additionally, nucleic acids that encode (or whose expression products encode) MuSK forms lacking a functional Ig2 domain may be introduced into cells ex vivo or in vitro. Still further alternatively or additionally, oligonucleotides that direct exon skipping of MuSK transcript(s) to favor forms that lack functional Ig2, or that direct degradation (or block translation) of forms that include functional Ig2, may be utilized.

In some embodiments, a population of cells is contacted with a MuSK Ig2 modulator agent and simultaneously or subsequently stimulated or expanded. Alternatively, or additionally, a population of cells is enriched or selected for cells exhibiting characteristics with early neurons, e.g., Dex, or mature neurons, e.g., Map2, astrocytes, e.g., GFAP and S100b, or oligodendrocytes, e.g., CNPase and 04.

In some embodiments, a resulting population of cells, achieved by contacting an original population of cells with a MuSK Ig2 modulator agent ex vivo is then administered to a subject. In some embodiments, a resulting population of cells is administered to a subject suffering from or susceptible to a disease or disorder such as a muscle disorder, neuromuscular dysfunction, a neurodegenerative disorder, a cardiac disorder (e.g., myocardial infarction, cardiomyopathy), or genetic diseases characterized by muscle wasting. In some embodiments, a resulting population of cells is administered to the subject from whom the original population of cells was obtained. In some embodiments, a resulting population of cells is administered to a different subject than the one from which the original population of cells was obtained; in some such embodiments, the original population was obtained from a healthy subject and the resulting population is administered to a subject suffering from or susceptible to a disease or disorder such as a muscle disorder, neuromuscular dysfunction, a neurodegenerative disorder, a cardiac disorder (e.g., myocardial infarction, cardiomyopathy), or genetic diseases characterized by muscle wasting.

In some embodiments, administering a population of cells, contacted with a MuSK Ig2 modulator agent effectively treats a disease or disorder such as a muscle disorder, neuromuscular dysfunction, a neurodegenerative disorder, a cardiac disorder (e.g., myocardial infarction, cardiomyopathy), or genetic diseases characterized by muscle wasting in the subject.

In some embodiments, a population of stimulated or expanded NSCs, SCs, MPCs, or myoblasts described herein can be formulated into a cellular therapeutic. In some embodiments, a cellular therapeutic includes a pharmaceutically acceptable carrier, diluent, or excipient. Pharmaceutically acceptable carriers described herein, for example, vehicles, adjuvants, excipients, and diluents, are well-known and readily available to those skilled in the art. Preferably, the pharmaceutically acceptable carrier is chemically inert to the active agent(s), e.g., a cellular therapeutic, and does not elicit any detrimental side effects or toxicity under the conditions of use.

In some embodiments, a cellular therapeutic can be formulated for administration by any suitable route, such as, for example, intravenous, intratumoral, intraarterial, intramuscular, intraperitoneal, intrathecal, epidural, or subcutaneous administration routes. Preferably, the cellular therapeutic is formulated for a parenteral route of administration. In some embodiments, a cellular therapeutic is administered to a subject via an infusion.

In some embodiments, a cellular therapeutic suitable for parenteral administration can be an aqueous or non-aqueous, isotonic sterile injection solution, which can contain anti-oxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

In some embodiments, a single therapeutic cell described herein is capable of expanding and providing a therapeutic benefit. In some embodiments, 10² or more, e.g., 10³ or more, 10⁴ or more, 10⁵ or more, or 10⁸ or more, therapeutic cells are administered as a cellular therapeutic. Alternatively, or additionally 10¹² or less, e.g., 10¹¹ or less, 10⁹ or less, 10⁷ or less, or 10⁵ or less, therapeutic cells described herein are administered to a subject as a cellular therapeutic. In some embodiments, 10²-10⁵, 10⁴-10⁷, 10³-10⁹, or 10⁵-10¹⁰ therapeutic cells described herein are administered as a cellular therapeutic.

A dose of a cellular therapeutic described herein can be administered to a subject at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of a cellular therapeutic may be administered in a single daily dose, or the total daily dosage may be administered in two, three, four, or more divided doses administered daily, as needed. In some embodiments, a cellular therapeutic is administered in combination with another therapy.

### Combination Therapy

In some embodiments, MuSK Ig2 modulator therapy as described herein is administered in combination with another therapy—i.e., so that a subject is simultaneously exposed to both therapies.

The dosage of the MuSK Ig2 modulator therapy as described herein and the dosage of another therapy administered in combination, as well as the dosing schedule can depend on various parameters, including, but not limited to, the disease being treated (e.g., a muscle disorder, neuromuscular dysfunction, a neurodegenerative disorder, a cardiac disorder, or a genetic disease characterized by muscle wasting), the subject's general health, and the administering physician's discretion.

MuSK Ig2 modulator therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other therapy, to a subject in need thereof. In various embodiments MuSK Ig2 modulator therapy and the other therapy are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, MuSK Ig2 modulator therapy and the other therapy are administered within 3 hours. In another embodiment, MuSK Ig2 modulator therapy and the other therapy are administered at 1 minute to 24 hours apart.

A synergistic combination of MuSK Ig2 modulator therapy and the other therapy, might allow the use of lower dosages of one or both of these agents or less frequent administration of the therapies to a subject suffering from muscle disorder, neuromuscular dysfunction, a neurodegenerative disorder, a cardiac disorder, or genetic diseases characterized by muscle wasting. A synergistic effect might result in the improved efficacy of these agents or the reduction of any adverse or unwanted side effects associated with the use of either agent alone.

In some embodiments, MuSK Ig2 modulator therapy is administered in combination with a standard of care treatment for a relevant disease, disorder, or condition (e.g., a muscle disorder, neuromuscular dysfunction, a neurodegenerative disorder, a cardiac disorder, or genetic diseases characterized by muscle wasting).

Therapies for DMD include deflazacort (Emflaza; PTC Therapeutics) eteplirsen (Exondys 51; Sarepta Therapeutics), Ataluren (Translarna; PTC Therapeutics), and glucocorticoids such as prednisone. In some embodiments, MuSK Ig2 modulator therapy is administered in combination with one or more therapies for DMD.

Approved therapies for ALS include Radicava, Rilutek, Tiglutik, and Nuedexta. In some embodiments, MuSK Ig2 modulator therapy is administered in combination with one or more therapies for ALS.

Approved therapies for cardiomyopathy include but are not limited to angiotensin II-converting enzyme (ACE) inhibitors, angiotensin II receptor blockers (ARBs) and spironolactone. In some embodiments, MuSK Ig2 modulator therapy is administered in combination with one or more therapies for cardiomyopathy.

In some embodiments, MuSK Ig2 modulator therapy is administered in combination with one or more therapies that relieves a symptom or characteristic of a relevant disease, disorder or condition, or of a therapy therefor. In some embodiments, MuSK Ig2 modulator therapy is administered in combination with one or more other therapies that relieves a symptom or characteristic so that the side effects associated with said other therapies are relieved. In some embodiments, the side effect associated with therapy is characterized by one or more of muscle cramps and spasms, constipation, fatigue, excessive saliva and phlegm, pain, depression, sleep problems, and uncontrolled outbursts of laughing or crying.

Any therapy which is known to be useful, or which has been used, will be used or is currently being used for the treatment or prevention of muscle disorder, neuromuscular dysfunction, a neurodegenerative disorder, a cardiac disorder, or genetic diseases characterized by muscle wasting, can be used in combination with the MuSK Ig2 modulator therapy in accordance with the invention described herein.

### Plasmids and Protein Production.

For preparing MuSK constructs of the present disclosure, including full-length MuSK and constructs comprising a deletion of one or more MuSK domains (e.g., Ig1, Ig2, or Ig3), plasmids can be used for packaging and expression of said constructs in a host cell.

In an exemplary embodiment, full-length ecto-domain MuSK cloned into a 6×His tag pCEP-Pu mammalian expression vector plasmid has been used in previous work to generate His-tagged full-length ecto-domain MuSK recombinant protein. Yilmaz et al. (2016). ΔIg1, ΔIg2, ΔIg1/2, Δ5a/5b, Ig1/2 MuSK ecto-domain deletion constructs were synthesized with NheI 5′ and NotI 3′ restriction enzyme recognition sites and cloned into a pUC57 bacterial expression vector (Genewiz). MuSK ecto-domain deletion constructs were cloned into the pCEP-Pu mammalian expression vector and Sanger sequenced for sequence confirmation. Isolated Ig1 and Ig2 isolated domains were generated by PCR cloning from full-length ectodomain MuSK CEP-Pu plasmid. PCR primers for Ig1:

(SEQ ID NO: 1)
forward GATCTTCTTTCTCCTTTGCCTGGC;
(SEQ ID NO: 2)
reverse TAAGCAGCGGCCGCCATCTTCACTTGCAGGGCACC
and
(SEQ ID NO: 3)
Ig2: forward CCTCAGCTAGCACCTAAAATAACTCGTCCTCCC;
(SEQ ID NO: 4)
reverse AATTAGCGGCCGCCTTCACCAGTTTGGAGTAAGC.

Isolated Ig1 and Ig2 PCR were cloned into the pCEP-Pu expression vector and Sanger sequenced for confirmation. For production of MuSK ecto-domain his-tagged proteins, Human Embryonic Kidney 293 (HEK 293) PEAKrapid cells (ATCC CRL-2828) were transfected with MuSK ecto-domain plasmids using Promega FugeneHD® and selected for by puromycin treatment. Ecto-domain MuSK-his recombinant protein is secreted into the media and conditioned media was collected every 2-3 days for two weeks. MuSK-his recombinant proteins were purified using nickel affinity chromatography from conditioned media. Purity of purified proteins was assessed by SDS-PAGE and quantified using ELISA and NanoDrop with calculated 0.1% extinction coefficients.

### MuSK PCR and Sequencing.

To amplify the MuSK exons expressed in vivo, RNA from wild-type soleus mouse muscle was isolated and reverse transcribed.

Primers that amplify the region of exons 5 to 8 were designed:

(SEQ ID NO: 7)
Forward: ACTGGTGAAGCTGGAAGTGG
(SEQ ID NO: 8)
Reverse: GCCTTGGCTGTCTTTCTGTG

PCR was performed using DreamTaq Green PCR master mix (ThermoFisher) and products were subjected to agarose gel electrophoresis to sufficiently separate PCR bands for gel extraction of DNA and to prevent cross-contamination of separated DNA bands. Extracted DNA was Sanger sequenced to identify the DNA sequence of each band.

### RNA-Sequencing and Bioinformatics.

RNA from wild-type and ΔIg3-MuSK mouse soleus was extracted using a RNeasy Fibrous Tissue mini kit (Qiagen) for RNA non-stranded library preparation and sequencing (Genewiz). Samples were sequenced at a depth of approximately 50 million reads per sample. Reads are assessed for quality and trimmed of adapter sequences using Trimmomatic then aligned with GSNAP to a reference genome. Read count matrices are generated using htseq-count. See Andrews, FastQC (2018); Bolger, Lohse, & Usadel (2014); Wu & Nacu (2010); Anders, Pyl, & Huber (2015). Read count matrices are uploaded to the integrated Differential Expression and Pathway (iDEP) analysis tool for exploratory data analysis, differential gene expression, and pathway GO analysis. Ge, Son, & Yao (2018). Read count matrices are used to run LeafCutter to identify and measure known and unknown alternative splicing events. See Li et al. (2018).

SEQUENCE LISTING

PCR primers for MuSK Ig1: Forward
GATCTTCTTTCTCCTTTGCCTGGC (SEQ ID NO: 1)
PCR primers for MuSK Ig1: Reverse
TAAGCAGCGGCCGCCATCTTCACTTGCAGGGCACC (SEQ ID NO: 2)
PCR primers for MuSK Ig2: Forward
CCTCAGCTAGCACCTAAAATAACTCGTCCTCCC (SEQ ID NO: 3)
PCR primers for MuSK Ig2: Reverse
AATTAGCGGCCGCCTTCACCAGTTTGGAGTAAGC (SEQ ID NO: 4)
MuSK HUMAN Ig3 Domain:
ARILRAPESHNVTFGSFVTLHCTATGIPVPTITWIENGNAVSSGSIQESV
KDRVIDSRLQLFITKPGLYTCIATNKHGEKFSTAKAAATIS
(SEQ ID NO: 5)
MuSK MOUSE Ig3 Domain
ARILRAPESHNVTFGSFVTLRCTAIGIPVPTISWIENGNAVSSGSIQESV
KDRVIDSRLQLFITKPGLYTCIATNKHGEKFSTAKAAATVS
(SEQ ID NO: 6)
Primers used to amplify MuSK cDNA: Forward.
ACTGGTGAAGCTGGAAGTGG (SEQ ID NO: 7)
Primers used to amplify MuSK cDNA: Reverse.
GCCTTGGCTGTCTTTCTGTG (SEQ ID NO: 8)

The following EXAMPLES are provided to illustrate the invention and shall not limit the scope of the invention.

### Example 1

This example provides constructs including the full-length (FL), MuSK with an Ig1 deletion (ΔIg1), MuSK with Ig2 deletion (ΔIg2) and MuSK with both Ig1 and Ig2 deletion (ΔIg1Ig2) were generated in order to study and characterize the binding properties of the various MuSK domains with BMP and its receptors.

### Materials and Methods

Antibodies and Materials. ALK2, ALK3, ALK4, ALK5, and ALK6 were obtained from R&D Systems (Minneapolis, MN, USA). Goat anti-Human IgG Fc Secondary Antibody, HRP (Cat #A18817) was obtained from ThermoFisher Scientific (Waltham, MA, USA). An expression vector containing His-tagged MuSK ectodomain was a gift from Dr. Markus Ruegg. Substrate 3,3′,5,5′-Tetramethylbenzidine (TMB) (Cat #T0440-100ML) was obtained from Sigma-Aldrich (St. Louis, MO, USA).

Production of Protein Constructs. The first step was to generate deletion constructs of the MuSK proteins. To map the domains by which MuSK binds to BMP receptors, the inventors generated deletion constructs of MuSK that are lacking a particular domain. the inventors used these deletion constructs to determine the domains of MuSK that binds BMP receptors.

The full-length of MuSK was sent to GENEWIZ (South Plainfield, NJ, USA), where the company manufactured a plasmid containing that sequence and returned it to the inventors. GENEWIZ provides us with the MuSK sequence in a pUC57 plasmid. The pUC57 plasmid is a commonly used plasmid for cloning vectors.

Subcloning Efficiency™ DH5a Competent Cells were used to transform the DNA acquired from GENEWIZ. The bacteria were then spread on an agar plate. The following day colonies were picked and added to tubes with broth. Throughout this process, ampicillin is being used as a form of selection. The colonies picked from the agar plates were left in LB media overnight.

The following day a miniprep was performed using a QIAprep Spin Miniprep Kit from QIAGEN (Venlo, Netherlands). Pellet 1-5 ml bacterial overnight culture by centrifugation at >8000 rpm (6800×g) for three minutes at room temperature (15-25° C.). Resuspend pelleted bacterial cells in 250 μL Buffer P1 and transfer to a microcentrifuge tube. Add 250 μL Buffer P2 and mix thoroughly by inverting the tube 4-6 times until the solution becomes clear. Do not allow the lysis reaction to proceed for more than five minutes. If using LyseBlue reagent, the solution will turn blue. Add 350 μL Buffer N3 and mix immediately and thoroughly by inverting the tube 4-6 times. If using LyseBlue reagent, the solution will turn colorless. Centrifuge for ten minutes at 13,000 rpm (˜17,900×g) in a table-top microcentrifuge. Apply the supernatant from step 5 to the QIAprep spin column by decanting or pipetting. Centrifuge for 30-60 s and discard the flow-through or apply vacuum to the manifold to draw the solution through the QIAprep spin column and switch off the vacuum source. Recommended: Wash the QIAprep spin column by adding 0.5 ml Buffer PB. Centrifuge for 30-60 s and discard the flow-through or apply vacuum to the manifold to draw the solution through the QIAprep spin column and switch off the vacuum source. (This step is only required when using endA+ strains or other bacteria strains with high nuclease activity or carbohydrate content.) Wash the QIAprep spin column by adding 0.75 ml Buffer PE. Centrifuge for 30-60 s and discard the flow-through or apply vacuum to the manifold to draw the solution through the QIAprep spin column and switch off the vacuum source. Transfer the QIAprep spin column to the collection tube. Centrifuge for one minute to remove residual wash buffer. Place the QIAprep column in a clean 1.5 ml microcentrifuge tube. To elute DNA, add 50 μL Buffer EB (10 mM Tris-Cl, pH 8.5) or water to the center of the QIAprep spin column, let stand for one minute, and centrifuge for one minute. For more information, please refer to the QIAprep Miniprep Handbook, (December 2006).

The concentration of this DNA obtained from this miniprep was then measured using a NanoDrop 2000/2000c from ThermoFisher Scientific.

The DNA obtained so far has the MuSK insert in the pUC57 vector. The next step was to move the MuSK insert into a pCEP4 Mammalian Expression Vector. Ampicillin is the prokaryotic selection form. Puromycin is used for eukaryotic selection. OriP allows the cells to take up the plasmid. EBNA-1 allows for the activation of viral DNA replication. BM40 is the secretion peptide that allows the cells to secret the protein into the media.

To complete the subcloning of the MuSK insert into the pCEP4 expression vector, restriction enzymes cut the MuSK insert out of the pUC57 vector. After the addition of the restriction enzymes, the DNA was run on an agarose gel to separate the MuSK insert from the rest of the vector. To extract the MuSK, insert from the gel, the inventors used a QIAquick extraction kit. To extract the MuSK, insert from the gel, the inventors used a QIAquick® Gel Extraction Kit (cat. nos. 28704, 28706, 28506 and 28115). Excise the DNA fragment from the agarose gel with a clean, sharp scalpel. Weigh the gel slice in a colorless tube. Add three volumes Buffer QG to one volume gel (100 mg gel ˜100 μL). The maximum amount of gel per spin column is 400 mg. For >2% agarose gels, add 6 volumes Buffer QG. Incubate at 50° C. for ten minutes (or until the gel slice has completely dissolved). Vortex the tube every 2-three minutes to help dissolve gel. After the gel slice has dissolved completely, check that the color of the mixture is yellow (similar to Buffer QG without dissolved agarose). If the color of the mixture is orange or violet, add 10 μL 3 M sodium acetate, pH 5.0, and mix. The mixture turns yellow. Add one gel volume isopropanol to the sample and mix. Place a QIAquick spin column in a provided 2 ml collection tube or into a vacuum manifold. To bind DNA, apply the sample to the QIAquick column and centrifuge for one minute or apply vacuum to the manifold until all the samples have passed through the column. Discard flow-through and place the QIAquick column back into the same tube. For sample volumes >800 μL, load and spin/apply vacuum again. If DNA will subsequently be used for sequencing, in vitro transcription, or microinjection, add 500 μL Buffer QG to the QIAquick column and centrifuge for one minute or apply vacuum. Discard flow-through and place the QIAquick column back into the same tube. To wash, add 750 μL Buffer PE to QIAquick column and centrifuge for one minute or apply vacuum. Discard flow-through and place the QIAquick column back into the same tube. Note: If the DNA will be used for salt-sensitive applications (e.g., sequencing, blunt-ended ligation), let the column stand two-five minutes after addition of Buffer PE. Centrifuge the QIAquick column in the provided 2 ml collection tube for one minute to remove residual wash buffer. Place QIAquick column into a clean 1.5 ml microcentrifuge tube. To elute DNA, add 50 μL Buffer EB (10 mM Tris-CI, pH 8.5) or water to the center of the QIAquick membrane and centrifuge the column for one minute. For increased DNA concentration, add 30 μL Buffer EB to the center of the QIAquick membrane, let the column stand for one minute, and then centrifuge for one minute. After the addition of Buffer EB to the QIAquick membrane, increasing the incubation time to up to four minutes can increase the yield of purified DNA. If purified DNA is to be analyzed on a gel, add 1 volume of Loading Dye to five volumes of purified DNA. Mix the solution by pipetting up and down before loading the gel.

After the DNA is extracted from the gel, ligation enzymes are used to ligate the MuSK insert into the pCEP4 vector. After subcloning, DH5a Competent Cells were used to transform the vector. The cells are streaked on an LB agar plate. The same process is then repeated for this newly formed vector. A miniprep is done in the same way, and the DNA concentration is again measured. All MuSK deletion constructs generated were sequenced out-of-house by GENEWIZ.

Transfection and Media Harvest. HEK293 cells were plated in T75 flasks at 70% confluency. Twenty-four hours later, DNA was transfected into growth media using FuGENE® HD. FuGENE® HD was obtained from Promega (Madison, WI, USA). Twenty-four hours post-transfection, growth media was changed to High Dose Antibiotic Selection media. Forty-eight hours post these media changes, the supernatant was harvested, and cells were put into Low Dose Antibiotic Selection Media.

To harvest media, media was spun down in sterile conical tubes to pellet cells. The supernatant was then transferred to a new sterile tube. The supernatants were frozen down with liquid nitrogen and then were stored at −80° C. until it was time to purify the protein.

Protein Purification. The reagents that were used for purification were: Nalgene Rapid-Flow filter Unit 0.45 um SFCA sterile filter (Fisher, Cat #156-0045), Amicon Ultra-15 Centrifugal Filter Devices (Fisher, Cat #UFC901024), Amicon Pro Affinity Concentration Kit—Ni-NTA (Fisher, Cat #ACK5010NT) and Gibco Distilled Water (Invitrogen, Cat #15230162).

The harvested media was left to thaw at 4° C. on a rocker overnight. Next, the growth media was filtered using the Nalgene Rapid-Flow filter Unit 0.45 um SFCA sterile filter. The Amicon Ultra-15 Centrifugal Filter Devices were then used to concentrate the growth media. The concentrated harvested media was then transferred to sterile tubes and stored on ice until it was time to purify them.

Nickel column affinity purification was used to purify the MuSK protein. Before usage, dialysis was performed to change the buffer and remove the imidazole from the purified protein. The final storage buffer is phosphate-buffered saline, pH 7.4.

The protocol for the Amicon® Pro Affinity Concentration Kit—Ni-NTA (Catalog Nos. ACR5000NT, ACK5003NT, ACK5010NT, ACK5030NT, ACK5050NT, ACK5100NT) metal chelation affinity purification of His-tagged recombinant proteins was that provided by EMD Millipore.

Bead preparation. To ensure uniform suspension, vortex the Ni-NTA resin thoroughly before adding it to the device. Remove the collection tube cap and open the exchange device cap. Add 200 μL of resin slurry to the base of the exchange device. Close the exchange cap. Up to 500 μL packed resin (1000 μL slurry volume) may be added per device. Using wide-bore tips (Cat. No. 02-707-134, Fisher Scientific) for resin transfer is recommended. To remove storage buffer, centrifuge in a swinging bucket rotor at 1000 g×one minute. Add 500 μL of 1× Bind Buffer. Centrifuge at 1000 g×one minute.

Protein binding. Add 500 μL of sample to the exchange device. Up to nine mL of sample can be added. The volume loaded is determined by the target protein's expression level and resin's binding capacity. Incubate for 60 min at room temp with gentle agitation. Upright agitation on a plate shaker at low setting is recommended. End-over-end mixing, particularly with small volumes or for extended time, may cause substantial bead loss to the sides of the feeder tube. Centrifuge the device at 1000 g×one minute in a swinging bucket rotor. Recovery of the sample flowthrough from the 50 mL collection tube is optional. To ensure maximal protein capture, all resin is collected into solution before centrifugation. Add 1.5 mL of Wash Buffer. Centrifuge at 1000 g×one minute. Recovery of the wash fraction from the 50 mL collection tube is optional. Because of the large capacity of the exchange device, the volume of the wash can be increased for greater sample purity. There is no need for multiple wash steps.

Sample elution. Samples can be eluted without concentration by adding elution buffer and centrifuging (1000g×two minutes) directly into a clean 50 ml collection tube. Given the limited volume processing capacity of the AU-0.5 device, this protocol is recommended if elution volumes >1.5 ml are required.

Solid-Phase Binding Assay. To assay the binding between MuSK and the ALKs, the inventors used an ELISA-like solid-phase binding assay. The plates used for the solid-phase binding assays were from 96 well plates from Sigma (Cat #M9410-1CS). The first step of the process was to immobilize the MuSK constructs on the 96 well plates. 50 ul of MuSK at the concentration of 16 ug/ml was added to the 96 well plates in triplicates. The plate was covered and left to incubate overnight at 4° C. The following day, the plates were washed using PBS (of the desired pH). The wells were then left in Block (1% BSA) for an hour. After blocking, all the washing steps were performed using PBST (0.05% Tween 20 and PBS solution).

After adding Block, a series dilution of ALK was added. For all the assays, the ALK concentrations added were 3.125, 6.25, 12.5, 25, 50, and 100 nM. To detect the presence of ALK, Goat anti-Human IgG Fc Secondary Antibody, HRP was used as a concentration of 1:250. TMB was then added to detect the presence of HRP. The reaction time was recorded, and when the reaction produced a high enough signal, the reaction was quenched using sulfuric acid.

To quantify the signal produced from these well, a plate reader was used to measure the signal at 450 nm. Absorbances were then background subtracted and plotted on a curve using Prism8 software. Each data point represented the average for three replicates.

SDS-PAGE and PNGase F. SDS-PAGE is a method for separating proteins by electrophoresis. Kaulich et al. (2020). SDS (sodium dodecyl sulfate is used to denature the proteins before the use of a polyacrylamide gel to separate out the sample. Kaulich et al. (2020). SDS provides the sample with a negative charge that is important when running the gel using electrophoresis. SDS-PAGE confirms the purity of the samples. The samples were treated with PNGase F to assess the glycosylation of samples. PNGase F is an enzyme that releases the N-linked oligosaccharides.

To evaluate the purity of the MuSK constructs generated, the constructs were run on SDS-PAGE gels. 1 ug of the protein was added to each well of the gel. The Mini-PROTEAN® TGX™ Precast Gels from BioRad (Hercules, CA) were used for these gel assays. To stain the gels, AcquaStain Protein Gel Stain (Bulldog-Bio, Portsmouth, NH, USA) was used to stain the gels overnight.

To show that the protein constructs were properly glycosylated, PNGase F was incubated with the protein and then later ran side by side on the gel with constructs not treated with PNGase F. The protocol used to treat the sampled with PNGase F is that provided by New England Biolabs Ipswich, MA, USA). Typical reaction conditions are:

Denaturing Reaction Conditions: Combine 1-20 μg of glycoprotein, 1 μL of Glycoprotein Denaturing Buffer (10×) and H O (if necessary) to make a 10 μL total reaction volume. Denature glycoprotein by heating reaction at 100° C. for ten minutes. Chill denatured glycoprotein on ice and centrifuge ten seconds. Make a total reaction volume of 20 μL by adding 2 μL GlycoBuffer 2 (10×), 2 μL 10% NP-40 and 6 μL H O. PNGase F is inhibited by SDS, therefore NP-40 is added in the reaction mixture under denaturing conditions. Failure to include NP-40 into the denaturing protocol will cause loss of enzymatic activity. Add 1 μL PNGase F, mix gently. Incubate reaction at 37° C. for 1 hour. Analyze by method of choice. Note: The simplest method of assessing the extent of deglycosylation is by mobility shifts on SDS-PAGE gels.

Non-Denaturing Reaction Conditions: When deglycosylating a native glycoprotein it is recommended that an aliquot of the glycoprotein is subjected to the denaturing protocol to provide a positive control for the fully deglycosylated protein. The non-denatured reaction can then be compared to the denatured reaction to determine the extent of reaction completion. Combine 1-20 μg of glycoprotein, 2 μL of GlycoBuffer 2 (10×) and H O (if necessary) to make a 20 μL total reaction volume. Add 2-5 μL PNGase F, mix gently. Incubate reaction at 37° C. for 4-24 hours.

To deglycosylate a native glycoprotein, longer incubation time and more enzymes may be required. Analyze by method of choice. The simplest method of assessing the extent of deglycosylation is by mobility shifts on SDS-PAGE gels.

### Results

### SDS-PAGE for MuSK Constructs.

FIG. 4 confirms that the constructs for each construct were generated. The full-length construct showed a smear because the construct has several glycosylation sites. The full-length construct had a mass around the 75 kDa marker while the full-length treated with PNGase F had a molecular weight that is slightly lower than that is untreated. The band along the 30 kDa marker in the treated lane shows the PNGase F enzyme used for the treatment. The ΔIg1 construct in FIG. 4 showed a smear because the construct has several glycosylation sites. The ΔIg1 construct had a mass around lighter than that of FL, while the ΔIg1 treated with PNGase F had a molecular weight that is slightly lower than that of the untreated. The band along the 30 kDa marker in the treated lane shows the PNGase F enzyme used for the treatment.

The ΔIg2 construct in FIG. 4 showed a smear because the construct has several glycosylation sites. The ΔIg2 construct had a mass similar to that of the ΔIg1 but lighter than that of the FL. The ΔIg2 treated with PNGase F had a molecular weight that is slightly lower than that of the untreated. The band along the 30 kDa marker in the treated lane shows the PNGase F enzyme used for the treatment. The ΔIg1-Ig2 construct in FIG. 4 showed a smear because the construct has several glycosylation sites. The ΔIg1 construct had a mass around lighter than that of FL, while the ΔIg1 treated with PNGase F had a molecular weight that is slightly lower than that of the untreated. The band along the 30 kDa marker in the treated lane shows the PNGase F enzyme used for the treatment.

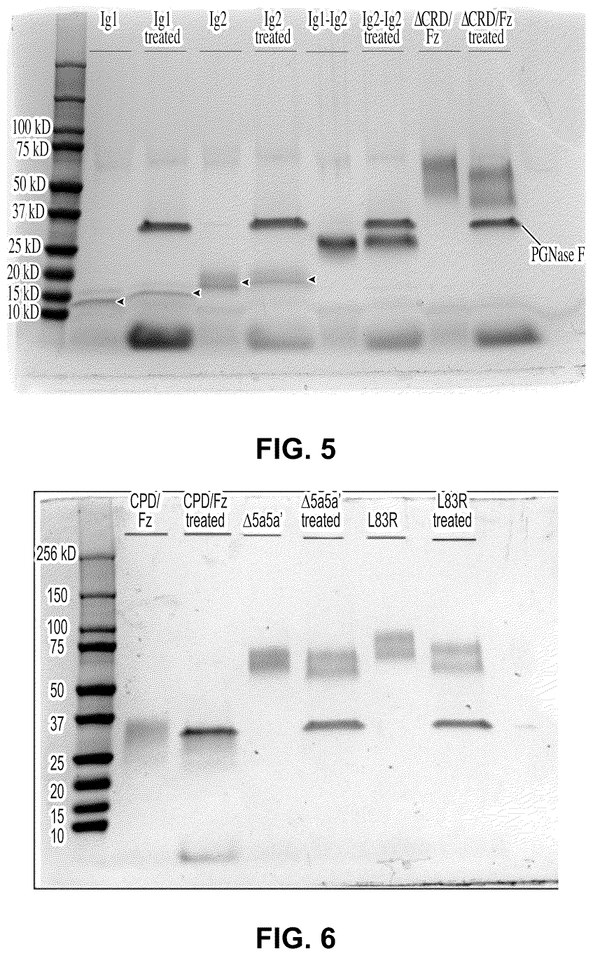

FIG. 5 shows the results for the Ig1, Ig2, Ig1-Ig2, and ACRD/Fz and confirms that the expected constructs were generated. The Ig1 band formed around 15 kDa. The Ig1 band treated with PNGase F appeared at the same marker as that of the untreated, which indicated that the Ig1 domain has no glycosylation sites. The band along the 30 kDa marker in the treated lane shows the PNGase F enzyme used for the treatment. In FIG. 5, the Ig2 band has a mass slightly over 15 kDa. The Ig2 band treated with PNGase F appeared at the same marker as that of the untreated, which indicated that the Ig2 domain has no glycosylation sites. The band along the 30 kDa marker in the treated lane shows the PNGase F enzyme used for the treatment.

In FIG. 5, the Ig1-Ig2 band appears to have a molecular weight of about 30 kDa. Th Ig1-Ig2 band shows no smear, and the mass is not affected after the treatment with PNGase F. The results indicated that the Ig1 and Ig2 domains are not glycosylated and have no glycosylation sites. The lone band along the 30 kDa marker in the treated lane shows the PNGase F enzyme used for the treatment. In FIG. 5, the ACRD/Fz smeared band appears around the 70 kDa mark. In FIG. 5, the band appears as a smear because the ACRD/Fz is glycosylation. This is further confirmed by the fact that the mass of the sample treated with PNGase F is smaller than that untreated. The lone band along the 30 kDa marker in the treated lane shows the PNGase F enzyme used for the treatment.

FIG. 6 shows the results for the CRD/Fz, the Δ5a5a′, and the L83R constructs. The CRD/Fz band in FIG. 6 is smeared, which shows some glycosylation. The PNGase F enzyme overlaps with the CRD/Fz band in the treated sample. 5a5a′ are alternatively spliced exons. The size of the bands shows that there is not much of a difference between this sample and the full-length sample. The smaller size of the treated sample shows that the sample has glycosylation sites. See the smeared band in FIG. 6. FIG. 6 also shows the L83R construct. MuSK L83R is significant because the leucine at the 83^{rd} position was shown to be necessary for MuSK dimerization. Stiegler, Burden, & Hubbard (2006). By mutating the leucine to arginine, the aim was to prevent MuSK from dimerizing. FIG. 6 shows MuSK L83R run on an SDS-PAGE gel. The bands are smeared because MuSK is glycosylated. The sole band along the 30 kDa marker in the treated lane shows the PNGase F enzyme used for the treatment.

### Example 2

### Ig2 Domain is Necessary and Sufficient for MuSK-BMP Receptor Binding

The inventors used ELISA-like solid-phase binding assays and the MuSK deletion constructs to map the domains of MuSK that bind the BMP receptors (ALKs). Yilmaz et al. (2016) previously showed that MuSK binds to ALK6 but did not determine which MuSK domains bind to ALK6.

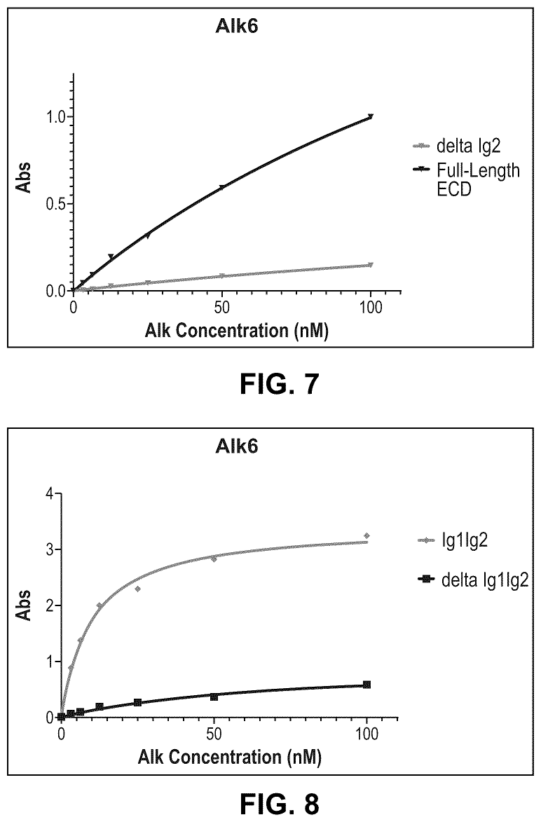

FIG. 7 shows the data from a solid phase binding assay where MuSK was immobilized and incubated with ALK6. The ALK concentration is shown on the x-axis and the absorbance is shown on the y-axis. The higher the absorbance, the better the binding. FIG. 7 shows that when the Ig2 domain is missing, MuSK is showing little to no binding compared to the full-length ectodomain (FL ECD) of MuSK. At an ALK6 concentration of 100 nM, FL ECD had an absorbance of 1, while the ΔIg2 MuSK construct had an absorbance of 0.15. In the presence of the Ig2 domain, MuSK increased by 6.6-fold. This increase is significant. FIG. 7 shows that the Ig2 domain is necessary for MuSK binding to ALK6. It is important to note the shape of the curve as it shows that there is not a high affinity for binding for the ΔIg2 curve.

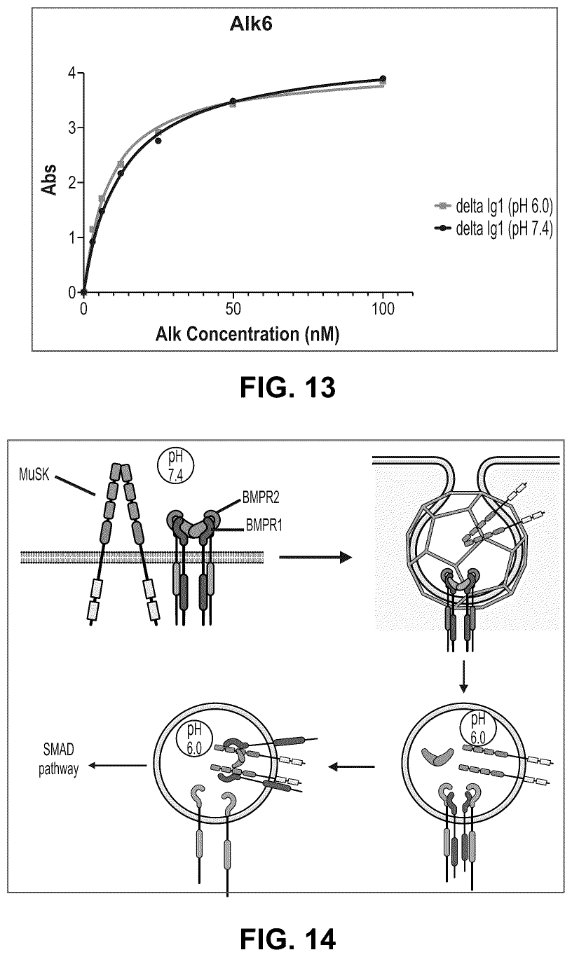

With the Ig2 domain necessary for binding, the next step was to isolate the binding to the Ig1-Ig2Ig1Ig2 region of the MuSK ectodomain. FIG. 8 shows the binding assay data from the Ig1-Ig2 construct and ΔIg1-Ig2 construct. FIG. 8 shows Ig1-Ig2 construct has a high-affinity binding to ALK6. The Ig1-Ig2 curve reaches an absorbance of 3 at a concentration of 100 nM. On the other hand, in the ΔIg1-Ig2 construct, MuSK shows little to no binding. This is even more evident when comparing it to the Ig1-Ig2 curve. The half max of the Ig1-Ig2 curve is at the 6.25 nM concentration, which shows that the Ig1-Ig2 construct has a high-affinity binding. FIG. 13 that Ig1 and the Ig2 domain are both necessary for binding MuSK to ALK6.

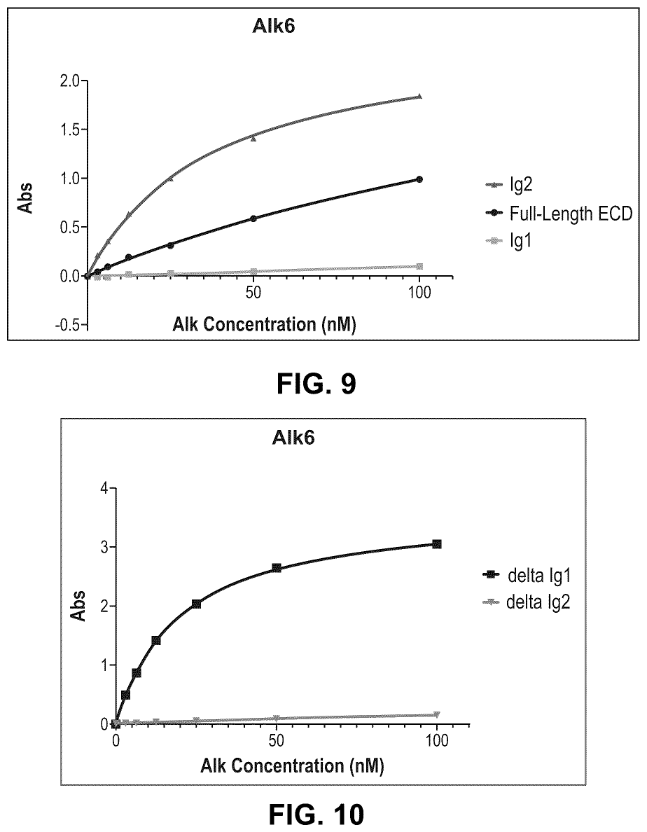

To show that the Ig2 domain is sufficient to bind ALK6, the inventors used isolated Ig2 and Ig1 domains in FIG. 9. FIG. 9 shows that the Ig2 curve has an absorbance curve of 1.8 and a half max concentration of 6.25 nM. The full-length MuSK in this assay did not saturate and had a half max only of about 0.5 nM. The isolated Ig1 domain curve showed little to no binding. These results indicate that the Ig2 domain is sufficient for binding as the Ig2 domain demonstrated a binding curve with high-affinity characteristics. FIG. 8 and FIG. 9 show that the Ig2 domain is necessary and sufficient for MuSK's binding to ALK6. Also see Table 1 which provides the half maximal and saturation levels for the binding assays. The full-length data were from Yilmaz et al. (2016). Saturation percentage measured at 100 nM.

TABLE 1

| | Half Max | Saturation |
|---|---|---|
| Musk construct | (nM) | (%) |
| FL (pH 7.4) | 12.5 ± 0.5* | 1 ± 0* |
| FL (ph 6.0) | TBD | TBD |
| ΔIg1 (pH 7.4) | 9.1 ± 1 | 0.99 ± 0 |
| ΔIg1 (pH 6.0) | 13.7 ± 3.6 | 1 ± 0 |
| ΔIg2 (pH 7.4) | no binding | |
| ΔIg2 (pH 6.0) | | |
| Ig1Ig2 (pH 7.4) | 18.2 ± 0.9 | 0.21 ± 0.07 |
| Ig1Ig2 (pH 6.0) | 15.9 ± 3.8 | 0.99 ± 0.01 |
| Ig1 (pH 7.4) | no binding | |
| Ig1 (pH 6.0) | | |
| Ig2 (pH 7.4) | TBD | |
| Ig2 (pH 6.0) | 23.6 ± 3.4 | 0.83 ± 0.05 |
| ΔIg1Ig2 (pH 7.4) | NA | NA |
| ΔIg1Ig2 (pH 6.0) | NA | NA |

### Example 3

### Ig1 Domain is not Necessary for MuSK Binding.

Ig1 domain is important as the domain at which MuSK binds to Agrin-LRP4.Stiegler, Burden, & Hubbard (2006). A crystal structure also shows that MuSK dimerizes via the Ig1 domain. Therefore, it was important to demonstrate the role Ig1 domain might have on the MuSK binding interaction to BMP. The MuSK ΔIg1 construct was immobilized on the solid-phase binding assay. FIG. 10 shows that MuSK binds strongly and with a high affinity to ALK6. The ΔIg1 curve shows a half max with a concentration of 12.5 nM. When compared to the ΔIg2 curve on the same FIG. 10, this result shows that the Ig1 domain is unnecessary for MuSK binding to ALK6. FIG. 10 shows that MuSK binding to ALK6 happens at the Ig2 domain location. While the solid-phase binding assay does not provide us with quantitative binding differences between the different constructs, the MuSK ΔIg1 construct had the highest absorbance compared to any construct that was assayed. Across several assays, the absorbance of the 100 nM ALK6 was always above 3. ΔIg1 has always produced high-affinity curves and seen in FIG. 10.

### Example 4

MuSK-ALK Binding is pH-Dependent

BMP co-receptors were shown to bind to BMP and BMP receptors in a pH-dependent manner. Yadin, Knaus, & Müller (2016). MuSK is a BMP co-receptor. The inventors have shown in this specification that MuSK binds to BMP receptors. The inventors further investigated whether MuSK binds to BMP receptors in a pH-dependent manner.

A structural review paper has shown that BMP receptors and BMP can bind to their binding partners in a pH-dependent manner. See Yadin, Knaus, & Müller (2016). A repulsive guidance molecule (RGM) is a protein that binds to BMP binds better at a slightly acidic pH. See Healey et al. (2015). RGM bind to BMP2 better at pH 5.5 compared to pH 7.4. Healey et al. (2015).

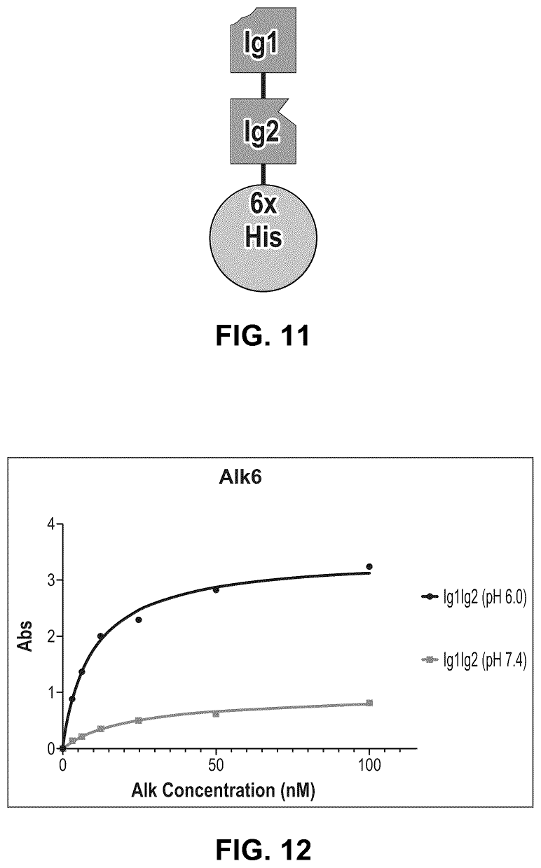

To assay the effect of pH on MuSK binding to BMP receptors, the Ig1-Ig2 only constructs were used. As shown in FIG. 11, the Ig1-Ig2 only constructs the MuSK ectodomain, where only the Ig1 domain and the Ig2 domain are present. FIG. 11 shows a schematic of this Ig1-Ig2 domain that was His-tagged for purification. Using a solid-phase binding assay, the inventors assayed if the pH affects MuSK binding to ALK6 (FIG. 12). FIG. 12 shows that MuSK binds significantly to ALK6 under slightly acidic conditions. At pH 6, the half max was 6.25 nM, while at pH 7.4, the half-maximum was at 25 nM. The difference in absorbance was significant, with the pH 6.0 sample reaching an absorbance of 3 while the pH 7.4 sample was barely reaching an absorbance of 0.75. This difference shows that MuSK prefers to bind to ALK6 at a slightly acidic condition. FIG. 12 also shows that at pH 6.0, MuSK is demonstrating a high-affinity curve with the first part of the curve rising quickly and then the rest of the curve plateauing around the absorbance of 3. This high-affinity curve shows that the interaction between MuSK and ALK6 at pH 6 is highly favorable at this condition compared to pH 7.0.

FIG. 13 shows that when the Ig1 domain is deleted, MuSK shows no pH dependence. FIG. 13 shows that ΔIg1 MuSK binds with a high affinity to ALK6 whether or not the pH is 6.0 or 7.4. This suggests that the Ig1 domain is the reason MuSK binds differently to ALK6 at pH 6 and pH 7. In FIG. 13, both curves reach a maximum absorbance of almost 4. The half max for both curves is at about 6.5 nM. The figures show a high-affinity curve with the first part of the curve rising quickly in absorbance.

### Example 5

While a full structure of the MuSK ectodomain does not yet exist, a crystal structure of MuSK Ig1-Ig2 alone is available. See Stiegler, Burden, & Hubbard (2006). The structure shows that Ig1 and Ig2 domains interact with one another. Stiegler, Burden, & Hubbard (2006). There are three points of contact between the Ig1 and the Ig2 domain Stiegler, Burden, & Hubbard (2006), two of which are van der Waals contacts, and one of which is a hydrogen bond between asparagine and valine. Stiegler, Burden, & Hubbard (2006). These Ig2 deletion constructs remove a domain in the middle of the protein. It is understood that the lack of MuSK binding to ALK6 in FIG. 7 could be due to the necessity of the Ig2 domain or that the entire protein was misfolded.

To determine how the Ig2 domain is critical for the interaction between MuSK and ALK6, the inventors designed and expressed an isolated Ig2 domain. FIG. 9 shows that an isolated Ig2 domain is binding to BMP receptors. This result from FIG. 7 and FIG. 9 shows that the Ig2 domain is necessary and sufficient for MuSK binding to BMP receptors. Ig1 domain has a role in binding MuSK's canonical binding partner, Agrin-LRP4. See Herbst & Burden (2000). Ig3 domain bind to BMP. See Yilmaz et al. (2016). Thus, the Ig2 domain binds to BMP receptors.

BMP and BMP receptors are known to have binding partners that bind in a pH-dependent manner. See Healey et al. (2015). To show that MuSK and BMP receptors bind in a pH-dependent manner, the inventors used the binding assay at a slightly acidic pH. Earlier assays were being performed at a pH of 7.4 but to assay the pH-dependence, the inventors assayed the binding affinity at a pH of 6.0. FIG. 12 shows that the Ig1-Ig2 construct at pH 6.0 binds much better and with a higher affinity at the lower pH compared to pH 7.4. There was no difference in binding affinity or absorbance at two different pHs when the ΔIg1 constructs were assayed. See FIG. 13. These results show that MuSK binds to BMP receptors in a pH-dependent manner. These results also show that the Ig1 domain is necessary for this pH dependence as the deletion of Ig1 removed any pH sensitivity to this interaction.

When there is pH dependence on protein interaction, the culprit residue that is causing this pH dependence is usually histidine. See Cui et al. (2020). Therefore, upon discovery of the pH-dependence binding by which MuSK binds to BMP receptors, it was important to check if there are any histidine residues that exist in the MuSK sequence. A histidine residue was found in the MuSK Ig2 domain. And from the crystal structure, the inventors can see that the histidine is exposed and is pointing outwards. This histidine residue could be playing a role in controlling how MuSK responds differently under two different pHs.

In this EXAMPLE, the histidine residue into alanine are mutated and the binding of the mutated constructs is assayed, to determine a lack of pH sensitivity in these constructs, or lack of binding together.

The binding of MuSK to ALK6 is favored at a slightly acidic pH. MuSK binds to ALK6 best when the Ig1 domain is deleted (FIG. 10). Several reasons can explain why MuSK binds strongly and with high affinity to ALK6 absent the Ig1 domain. Given that MuSK binds to ALK6 through the Ig2 domain, the deletion of the Ig1 domain could be removing a steric hindrance that BMP receptors are subject to when binding. Ig1 domain could be slightly blocking the binding interface at the Ig2 domain, and therefore the ΔIg1 constructs bind significantly better than any other construct. Ig1 domain is necessary for MuSK dimerization, as shown in the crystal structure of Ig1-Ig2 domains. See Stiegler, Burden, & Hubbard (2006). See FIG. 19.

### Example 6

### A Working Model for how MuSK Binds to BMP Receptors in the Context of BMP Signaling.

On the surface of the cell, the pH is 7.4, BMP is bound to BMP receptors, and MuSK dimerizes using the Ig1 domain. See Stiegler, Burden, & Hubbard (2006); Yadin, Knaus, & Müller (2016); and Lardner (2001). The first panel in FIG. 14 shows MuSK as a dimer and BMP receptors canonically binding to BMPs. An event in the cell then triggers clathrin-mediated endocytosis, which is shown in the next panel of FIG. 14. For the cell to initiate clathrin-mediated endocytosis, the accumulation of PIP2 and adaptor proteins is necessary. Kaksonen & Roux (2018). Clathrin-mediated endocytosis leads to the formation of endosomes. Endosomes are known to have a slightly acidic pH compared to the extracellular environment of cells. Geisow & Evans (1984) BMP receptors type 1 favors binding MuSK at these conditions. In FIG. 14, the lower pH should cause the BMP to also dissociate from BMP receptors and bind to the BMP receptor, but no experimental data has shown this yet. BMP receptors are known to be present in endosomes. Hartung et al. (2006). The MuSK-BMP and MuSK-BMP receptor pathways are likely leading to the phosphorylation of SMAD proteins. Endosomes and the MuSK-BMP pathway thus create an enhanced SMAD pathway for the cell.

This disclosure shows that the Ig2 domain binds to ALK6. NMR is being planned to specify the residues of the Ig2 domain by which MuSK binds to BMP receptors. The pH dependence shows that a histidine in the Ig2 domain is important for the interaction. The Ig2 domain is about 15 kDa. The inventors are planning to express the protein in E. Coli and use NMR to identify in more detail what are the residues that get perturbed when the Ig2 domain binds to ALK6. Given that MuSK ECD binds best when the Ig1 domain is deleted and given that Ig1 has three contacts with the Ig2 domain, the perturbed residues could be those points of contact. See Stiegler, Burden, & Hubbard (2006).

MuSK likely binds to BMP receptors primarily in endosomes. The inventors are planning to disrupt clathrin-mediated endocytosis in cells and observe if the SMAD pathway is changing as a result. The inventors are using the anti-emetic/anti-psychotic prochlorperazine as it was shown to inhibit the in vivo endocytosis of membrane proteins. See Chew et al. (2020). Because inhibiting the endocytosis affects the SMAD pathway and on the phosphorylation of the cells, MuSK and BMP receptors signal through the endocytosis.

The role that MuSK dimerization or monomerization has on the binding to BMP receptors. As shown in FIG. 10, MuSK binds the best BMP receptors when the Ig1 domain is deleted. It is also apparent from the crystal structure that MuSK dimerizes via the Ig1 domain. MuSK can likely dimerize and monomerize to regulate binding to BMP receptors. The inventors are planning to use size-exclusion chromatography to assess if the Ig1 samples is a monomer and if the other samples are dimers. Size exclusion chromatography can be used to assay whether MuSK is a dimer at the pH of 6.0. MuSK could be dimerizing at pH 7.4 and is a monomer at a pH of 6.0, and therefore MuSK binds better at that pH.

### Example 7

### RNA Analysis

The inventors used 5-month old Pax7^{CreERT2};TdTomato^{FI/FI} mice for this EXAMPLE. See Pawlikowski et al. (2015). Mouse transverse abdominis (TA) muscles were injured using an injection of 50 μL of 1.2% BaCl₂. Four days after injury, transverse abdominis muscles were harvested and then digested and minced using 4000U/mL collagenase II for ninety minutes, followed by a series of cell filtrations. Satellite cells (SCs) were then isolated from the cell suspension by FACS sorting, using TdTomato expression and antibody against integrin apha-7 as positive cell-selection markers and antibodies against CD45, CD31, and Ly-6aA/E as negative cell-selection markers. RNA was isolated from satellite cells using Qiagen RNeasy mini kit. cDNA was generated using AzuraQuant cDNA synthesis kit. The following PCR primers were used to amplify MuSK cDNA: forward: ACTGGTGAAGCTGGAAGTGG (SEQ ID NO: 7) and Reverse: GCCTTGGCTGTCTTTCTGTG (SEQ ID NO: 8). PCR products were run on 3% agarose, 0.004% EtBr gel in TBE.

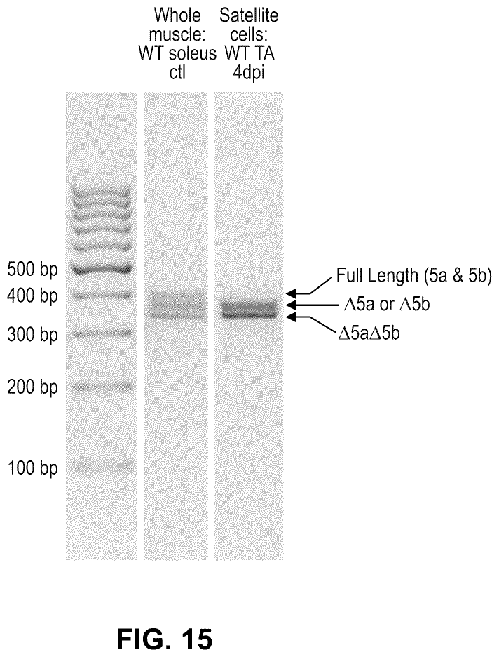

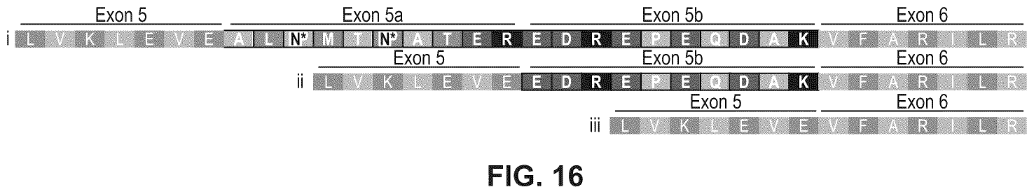

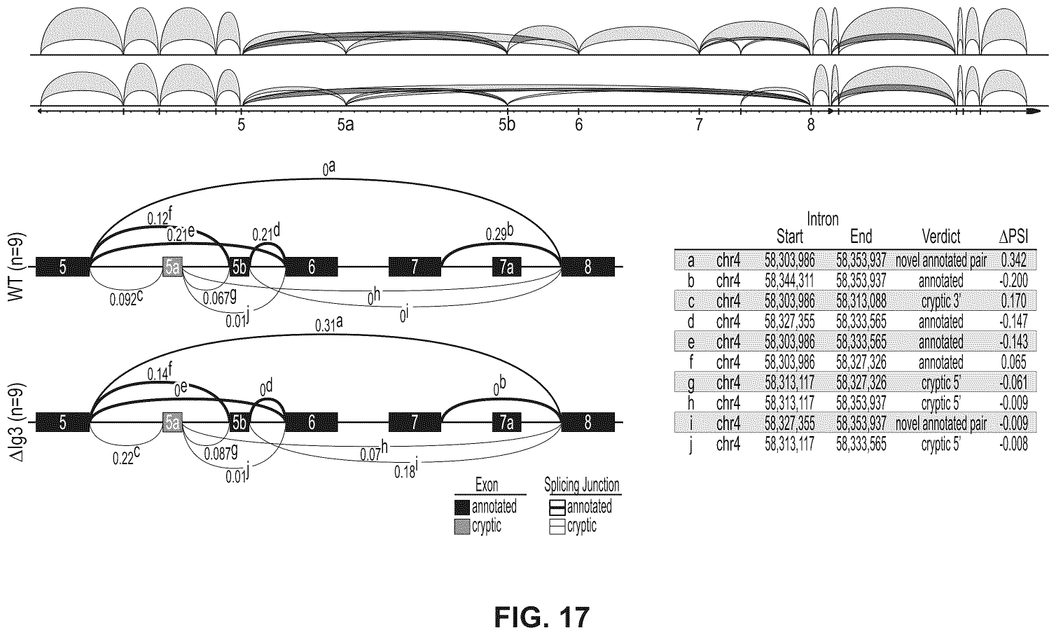

The results are shown in FIG. 15. The amino acid sequence of the MuSK containing alternatively spliced exons 5a and 5b is provided in FIG. 16. FIG. 17 shows alternatively splicing patterns of exons 5a and 5b from mouse soleus muscle.

### Example 8

### MuSK Ig2 Modulator Oligonucleotides

Design and synthesize of exon-skipping ASOs. Without wishing to be bound by any theory, MuSK MR agonizing oligonucleotides as described herein are designed in accordance with, but not limited to, the following general guidelines (see, Aartsma-Rus et al., Humana Press, 2012, 117-129):
RNA or DNA modified for resistance to endo- or exonucleases (e.g., 2′MoE, 2′OMe, PMO, phosphorthioate);
Designed against target sequence;
Typically between 15-25 nucleotides, more optimally between 17-20;
Typically most effective with melting temperature of over 48° C.;
Typically most effective with GC content between 40% and 60% to prevent steric hindrance/dimerization, availability to access target;
Typically most effective targeting open/accessible pre-mRNA structures;
Typically most effective targeting splice regulatory sites or exon definition sites (e.g., intronic splice enhancers, intronic splice silencers (e.g., Spinraza, targeted against ISS of SMN2 exon 7, exonic splice enhancers, exonic splice silencers);
Typically most effective with sequence composition containing no more than 2 guanine (G) or cytosine (C) nucleotides in direct succession (e.g., CCC or GGG).

ASO chemistry. We will develop 2′-O—2-methoxyethyl (2′MOE) ASOs that also include phosphorothioate bonds in the sugar backbone. Methods for the design and testing of such ASOs are well-established, including manufacturing, pharmacokinetics, biodistribution and toxicology in rodents and non-human primates (Bennett and Swayze, 2010; Chiriboga et al., 2016; Hua et al., 2015; Mercuri et al., 2018; Rigo et al., 2014). The MOE group added to the 2′ position of the ribose increases Tm by about 2° C. per residue, thus elevating the binding affinity, and also improving nuclease resistance. The phosphorothioate modification confers further nuclease resistance and also increases affinity for plasma proteins, resulting in ASOs that efficiently distribute to tissues and are taken up into cells with need for formulations. This chemistry is off-patent, offering commercial advantages.

ASO Design. ASOs will be synthesized by a commercial facility and will be provided by Bolden Therapeutics, Inc. to the Fallon and Webb labs at Brown University for screening. The strategy for designing the ASOs will include scanning exonic and intronic sequences flanking both the 5′ and 3′ splice sites with overlapping ASOs (1-2 bp shift/oligo). The optimal length of the ASOs is ˜17mer which provides a good balance between target specificity and drug exposure. ASOs will be pre-screened in silico for potential off-target effects as well as compositional bias (GC content) and propensity for unwanted dimer formation.

The ASOs will be designed such that they induce skipping of both exons 5a and 5b in MuSK.

### Screening and Selection of Optimal Exon-Skipping a SOs.

We will design RT-qPCR TaqMan assays to specifically quantify the following distinct MuSK splice forms: 1) full length (FL) MuSK; 2) Δexon^{5a-5b}, which is the desired product encoding ΔIg2-MuSK; and 3) potential ‘incomplete’ skipping’ isoforms Δexon^{5a} and Δexon^{5b}. We will perform conventional RT-PCR in parallel to detect any unexpected products. Cells will be transfected with candidate ASOs at several concentrations across a range from ˜0.1 to 10 nM. After one day of treatment RNA will be extracted and splicing will be measured by RT-qPCR to assess exon-skipping efficiency. Our goal is to isolate at least one ASO that induces ≥80% coordinate splicing of exons 5a and 5b.

Testing the Selected ASOs for their Ability to Inhibit MuSK-BMP Receptor Signaling in Cultured Cells.

We have observed that the ΔIg2 domain of MuSK is necessary for MuSK-BMP receptor binding. However, since skipping in vivo will likely be less than 100% efficiency (Rigo et al., 2014), it is important to establish the relationship between the level of skipping achieved and the physiological impact. Therefore, in this aim we will measure the level of MuSK-BMP receptor (ALK) dependent signaling in ASO-treated cells.

We will use qRT-PCR to measure the levels of MuSK-BMP receptor dependent transcripts. Cells treated with either exon-skipping of control ASOs will be stimulated with ALK for 2 hr. The levels of transcripts will then be measured and the response to BMP will be correlated with the degree of exon skipping.

Data. The present disclosure appreciates that, in some embodiments, efficiency of skipping exons 5a and 5b may be insufficiently efficient with a single ASO. In some embodiments, it may be desirable to prepare one or more ASOs directed against exon 5b, for use alone or with an exon 5a-directed ASO.

Those of ordinary skill in the art, reading the present disclosure, will appreciate that, in some embodiments, it may be desirable to replicate studies (e.g., at least three times), or to analyze data with appropriate statistical methodologies (e.g., by t-tests with appropriate correction for multiple comparisons (e.g. Bonferroni)).

Work described herein provides technologies for efficient development of an ASO-mediated therapy for neuromuscular diseases and disorders and enhance muscle regeneration.

### LIST OF REFERENCES

A person of ordinary skill in the biomedical art can use these patents, patent applications, and scientific references as guidance when making and using the invention.

### PATENT LITERATURE

U.S. Pat. No. 9,574,015 B2 (Burden et al.). This patent discloses a method of treating a subject suffering from a neuromuscular disorder, i.e., a method for improving motor function in a subject afflicted with muscle specific receptor kinase myasthenia gravis (MuSK-MG), by administering a portion of a MuSK polypeptide, i.e., administering a muscle specific receptor kinase (MuSK) first immunoglobulin-like domain 1 (Ig1). Burden does not disclose the method is for increasing neurogenesis, the method comprising a step of or the specific steps of increasing level or activity of a MuSK polypeptide lacking a functional Ig3 domain; or reducing level or activity of a BMP-MuSK polypeptide complex, wherein the MuSK comprises a functional Ig3 domain.

WO 2012/109075 A1 (Plexxikon Inc.). This patent publication discloses a step of modulating the activity of MuSK using kinases and compounds that selectively modulate kinases and uses for disease indications with features of neurodegeneration or impaired cognition (para [0001]) that are amenable to treatment by modulation of kinase activity (para. [0003]), for example, the Alzheimer's neurodegenerative disease. The compounds include Formula I (disclosed) and all sub-generic formulae, compounds as recited in the claims, and compounds described in this specification that are modulators of protein kinases. Disease indications amenable to treatment by modulation of kinase activity by the compounds described. This patent publication does not disclose increasing level or activity of a MuSK polypeptide lacking a functional Ig3 domain; or reducing level or activity of a BMP-MuSK polypeptide complex, wherein the MuSK comprises a functional Ig3 domain. This patent publication does not disclose using this in a method of increasing neurogenesis or increasing level or activity of a MuSK polypeptide lacking a functional Ig3 domain; or reducing level or activity of a BMP-MuSK polypeptide complex, wherein the MuSK comprises a functional Ig3 domain.

WO 2020/214987 A1 (Brown University) Neurogenesis, discloses methods and compositions for increasing neurogenesis and for preventing or treating diseases, disorders or conditions associated with neurodegeneration.

### Non-Patent Literature

Aartsma-Rus, Overview on DMD exon skipping. Methods Mol. Biol. 867, 97-116 (2012).
Allen et al., Cell proliferation is reduced in the hippocampus in schizophrenia. Australian & New Zealand Journal of Psychiatry, 50.5, 473-480 (2016).
Allen, Ligand-targeted therapeutics in anticancer therapy. Nature Reviews Cancer, 2, 750-765 (2002).
Allison et al., Comparison of different MRI-based morphometric estimates for defining neurodegeneration across the Alzheimer's disease continuum. Neuroimage Clin., 23, 101895 (2019).
Amenta et al., Biglycan recruits utrophin to the sarcolemma and counters dystrophic pathology in mdx mice. Proc. Natl. Acad. Sci. U.S.A., 108, 762-767 (2011).
Amenta et al., Biglycan is an extracellular MuSK binding protein important for synapse stability. J. Neurosci., 32, 2324-2334 (2012).
Anders, Pyl, & Huber, HTSeq—a Python framework to work with high-throughput sequencing data. Bioinformatics, 31, 166-169 (2015).
Andrews. FastQC: A Quality Control Tool for High Throughput Sequence Data (2010).
Atkinson et al., No Evidence for Recent Selection at FOXP2 among Diverse Human Populations. Cell, 174, 1424-1435.e15 (2018).
Audesse & Webb, Enhancing Lysosomal Activation Restores Neural Stem Cell Function During Aging. J. Exp. Neurosci., 12, 1179069518795874 (2018).
Barrés-Loscertales et al., Reduced striatal volume in cocaine-dependent patients. Neuroimage, 56.3, 1021-1026 (2011).
Bennett & Swayze, RNA targeting therapeutics: molecular mechanisms of antisense oligonucleotides as a therapeutic platform. Annu. Rev. Pharmacol. Toxicol., 50, 259-293 (2010).
Bennett et al., Antisense Oligonucleotide Therapies for Neurodegenerative Diseases. Annu. Rev. Neurosci. 42, 385-406 (2019).
Berge et al., Pharmaceutical salts. J. Pharm. Sci., 66(1), 1-19 (1977).
Blurton-Jones et al., Neural stem cells improve cognition via BDNF in a transgenic model of Alzheimer disease. Proc. Natl. Acad. Sci. U.S.A., 106, 13594-13599 (2009).
Boldrini et al., Human Hippocampal Neurogenesis Persists throughout Aging. Cell Stem Cell, 22, 589-599.e5 (2018).
Bolger, Lohse, & Usadel, Trimmomatic: a flexible trimmer for Illumina sequence data. Bioinformatics, 30, 2114-2120 (2014).
Bragdon et al., Bone morphogenetic proteins: a critical review. Cell Signal., 23, 609-620 (2011).
Burden et al., The role of MuSK in synapse formation and neuromuscular disease. Cold Spring Harb. Perspect. Biol., 5, a009167 (2013). Burden does not disclose the method is for treating a subject suffering from one or more features of neurodegeneration or impaired cognition, the method comprising a step of or the specific steps of increasing level or activity of a MuSK polypeptide lacking a functional Ig3 domain; or reducing level or activity of a BMP-MuSK polypeptide complex, wherein the MuSK comprises a functional Ig3 domain.
Camicioli et al., Parkinson's disease is associated with Hippocampal Atrophy. Mov. Disord., 18(7), 784-790 (2003).
Chan et al., Hippocampal volume in vulnerability and resilience to depression. Journal of Affective Disorders, 189, 199-202 (2016).
Chen et al., Enhancement of hippocampal neurogenesis by lithium. Journal of Neurochemistry, 75.4, 1729-1734 (2000).
Chen et al., Allopregnanolone promotes regeneration and reduces β-amyloid burden in a preclinical model of Alzheimer's disease. PLoS One, 6, e24293 (2011).
Chew et al., Endocytosis Inhibition in Humans to Improve Responses to ADCC-Mediating Antibodies. Cell, 180, 895-914.e27 (2020).
Chiriboga et al., Results from a phase 1 study of nusinersen (ISIS-SMN(Rx)) in children with spinal muscular atrophy. Neurology, 86, 890-897 (2016).
Choi et al., Effect of exercise-induced neurogenesis on cognitive function deficit in a rat model of vascular dementia. Molecular Medicine Reports, 13A, 2981-2990 (2016).
Choi et al., Combined adult neurogenesis and BDNF mimic exercise effects on cognition in an Alzheimer's mouse model. Science, 361 (2018).
Cook et al., Hippocampal volumetric and morphometric studies in frontal and temporal lobe epilepsy. Brain, 115.4, 1001-1015 (1992).
Crews et al., Increased BMP6 levels in the brains of Alzheimer's disease patients and APP transgenic mice are accompanied by impaired neurogenesis. J. Neurosci., 30, 12252-12262 (2010).
Cui et al., Mapping the structural and dynamic determinants of pH-sensitive heparin binding to granulocyte macrophage colony stimulating factor. Biochemistry, 59, 3541-3553 (2020).
Brandhorst et al., A Periodic Diet that Mimics Fasting Promotes Multi-System Regeneration, Enhanced Cognitive Performance, and Healthspan. Cell Metab., 22, 86-99 (2015).
Das, Debjani et al., Lifetime cigarette smoking is associated with striatal volume measures. Addiction biology, 17.4, 817-825 (2012).
De Chiara et al., The receptor tyrosine kinase MuSK is required for neuromuscular junction formation in vivo. Cell, 85, 501-512 (1996).
Dix & Aggleton, Extending the spontaneous preference test of recognition: evidence of object-location and object-context recognition. Behav. Brain Res., 99, 191-200 (1999).
Ekonomou et al., Increased neural progenitors in vascular dementia. Neurobiology of Aging, 32.12, 2152-2161 (2011).
Enwere et al., Aging results in reduced epidermal growth factor receptor signaling, diminished olfactory neurogenesis, and deficits in fine olfactory discrimination. J. Neurosci., 24, 8354-8365 (2004).
Eriksson et al., Neurogenesis in the adult human hippocampus. Nature Med., 4, 1313-1317 (1998).
Ernst et al., (2014). Neurogenesis in the striatum of the adult human brain. Cell, 156, 1072-1083 (2014).
Ersche et al., Meta-analysis of structural brain abnormalities associated with stimulant drug dependence and neuroimaging of addiction vulnerability and resilience. Current opinion in neurobiology, 23.4, 615-624 (2013).
Farrar et al., Emerging therapies and challenges in spinal muscular atrophy. Ann. Neurol., 81, 355-368 (2017).
Fichtner et al., Autoimmune pathology in myasthenia gravis disease subtypes is governed by divergent mechanisms of immunopathology. Front. Immunol. 11, 776 (2020).
Fish & Fallon, Multiple MuSK signaling pathways and the aging neuromuscular junction. Neurosci. Lett., 731, 135014 (2020).
Foland et al., Increased volume of the amygdala and hippocampus in bipolar patients treated with lithium. NeuroReport, 19.2, 221 (2008).
Gage, Adult neurogenesis in mammals. Science, 364, 827-828 (2019).
Garcia-Osta et al., MuSK expressed in the brain mediates cholinergic responses, synaptic plasticity, and memory formation. J. Neurosci., Off. J. Soc. Neurosci., 26, 7919-7932 (2006).
Ge, Son, & Yao, iDEP: an integrated web application for differential expression and pathway analysis of RNA-Seq data. BMC Bioinformatics, 19, 534 (2018).
Geisow & Evans, pH in the endosome: Measurements during pinocytosis and receptor-mediated endocytosis. Exp. Cell Res., 150, 36-46 (1984).
Gold et al., Hippocampal damage and memory impairments as possible early brain complications of type 2 diabetes. Diabetofogia, 50.4, 711-719 (2007).
Guptill, Sanders & Evoli, Anti-MuSK antibody myasthenia gravis: Clinical findings and response to treatment in two large cohorts. Muscle Nerve, 44, 36-40 (2011).
Hartung et a., Different Routes of Bone Morphogenic Protein (BMP) Receptor Endocytosis Influence BMP Signaling. Mol. Cell Biol., 26, 7791-7805 (2006).
Healey et al., Repulsive guidance molecule is a structural bridge between neogenin and bone morphogenetic protein. Nature Struct. Mol. Biol., 22, 458-465 (2015).
Herbst & Burden, The juxtamembrane region of MuSK has a critical role in agrin-mediated signaling. EMBO J 19: 67-77 (2000).
Hesser et al., Identification and characterization of a novel splice variant of MuSK. FEBS Lett., 442, 133-137 (1999).
Ho et al., Effects of diabetes on hippocampal neurogenesis: links to cognition and depression. Neuroscience & Biobehavioral Reviews, 37.8, 1346-1362 (2013).
Hong et al., CCL2 induces neural stem cell proliferation and neuronal differentiation in Niemann-Pick type C mice. Journal of Veterinary Medical Science, 14-0352 (2015).
Hua et al., Motor neuron cell-nonautonomous rescue of spinal muscular atrophy phenotypes in mild and severe transgenic mouse models. Genes Dev. 29, 288-297 (2015).
Hubbard & Gnanasambandan, Structure and activation of MuSK, a receptor tyrosine kinase central to neuromuscular junction formation. Biochim. Biophys. Acta, 1834, 2166-2169 (2013).
Imayoshi et al., Roles of continuous neurogenesis in the structural and functional integrity of the adult forebrain. Nat. Neurosci. 11, 1153-1161 (2008).
James et al., A review of the clinical side effects of bone morphogenetic protein-2. Tissue Eng. Part B Rev., 22, 284-297 (2016).
Jayam Trouth et al., Autoimmune diseases, myasthenia gravis: A review (2012).
Kaksonen & Roux, Mechanisms of clathrin-mediated endocytosis. Nature Rev. Mol. Cell Biol., 19, 313-326 (2018).
Kalamakis et al., Quiescence modulates stem cell maintenance and regenerative capacity in the aging brain. Cell 176, 1407-1419.e14 (2019).
Kantarci et al., Hippocampal volumes predict risk of dementia with Lewy bodies in mild cognitive impairment. Neurology, 87.22, 2317-2323 (2016).
Kathner-Schaffert et al., Early stroke induces long-term impairment of adult neurogenesis accompanied by hippocampal-mediated cognitive decline. Cells, 8 (2019).
Kaulich et al., Complementarity of different SDS-PAGE gel staining methods for the identification of short open reading frame-encoded peptides. Proteomics, 20, 2000084 (2020).
Kempermann et al., Human adult neurogenesis: evidence and remaining questions. Cell Stem Cell, 23, 25-30 (2018).
Kim et al., LRP4 is a receptor for agrin and forms a complex with MuSK. Cell, 135, 334-342 (2008).
Koh & Park, Neurogenesis in stroke recovery. Transl. Stroke Res., 8, 3-13 (2017).
Lardner, The effects of extracellular pH on immune function. J. Leukoc. Biol., 69, 522-530 (2001).
Leeman et al., Lysosome activation clears aggregates and enhances quiescent neural stem cell activation during aging. Science 359, 1277-1283 (2018).
Li et al. Annotation-free quantification of RNA splicing using LeafCutter. Nature Genet., 50, 151-158 (2018).
Li et al., Hippocampal subfield volumetry in patients with subcortical vascular mild cognitive impairment. Scientific Reports 6: 20873 (2016).
Lindvall & Kokaia, Neurogenesis following stroke affecting the adult brain. Cold Spring Harbor Perspectives in Biology, 7(11), p.a019034 (2015).
Lu et al., Targeting adult neurogenesis for poststroke therapy. Stem Cells International (2017).
Lucassen et al., Adult neurogenesis, human after all (again): Classic, optimized, and future approaches. Behav. Brain Res. 112458 (2019).
Mak et al., Multi-modal MRI investigation of volumetric and microstructural changes in the hippocampus and its subfields in mild cognitive impairment, Alzheimer's disease, and dementia with Lewy bodies. International Psychogeriatrics, 29.4, 545-555 (2017).
Mann et al., Antisense-induced exon skipping and synthesis of dystrophin in the mdx mouse. Proc. Natl. Acad. Sci. U.S.A., 98, 42-47 (2001).
McClorey et al., Antisense oligonucleotide-induced exon skipping restores dystrophin expression in vitro in a canine model of DMD. Gene Ther., 13, 1373-1381 (2006).
Mercado et al., Biglycan targets dystrobrevin, syntrophin and nNOS to the muscle cell membrane. FASEB J. (2006).
Mercuri et al., Nusinersen versus sham control in later-onset spinal muscular atrophy. N. Engl. J. Med. 378, 625-635 (2018).
Meyers & Miller Optimal alignments in linear space. CABIOS, 4: 11-17 (1989).
Mira et al., Signaling through BMPR-IA regulates quiescence and long-term activity of neural stem cells in the adult hippocampus. Cell Stem Cell 7, 78-89 (2010).
Moreno-Jimenez et al., Adult hippocampal neurogenesis is abundant in neurologically healthy subjects and drops sharply in patients with Alzheimer's disease. Nature Medicine (2019).
Morris, Epitope mapping protocols, Methods in Molecular Biology, 66 (1996).
Mueller, RGM co-receptors add complexity to BMP signaling. Nature Struct. Mol. Biol., 22, 439-440 (2015).
Mullane & Williams Alzheimer's disease (AD) therapeutics-1: Repeated clinical failures continue to question the amyloid hypothesis of AD and the current understanding of AD causality. Biochem. Pharmacol., 158, 359-375 (2018).
Murphy & Ramakrishnan, Structure of a purine-purine wobble base pair in the decoding center of the ribosome. Nature Structural and Molecular Biology, 11, 1251-1252 (2004).
Myasthenia gravis I Genetic and Rare Diseases Information Center (GARD)—an NCATS Program.
Nathan et al., Association between CSF biomarkers, hippocampal volume and cognitive function in patients with amnestic mild cognitive impairment (MCI). Neurobiology of Aging, 53, 1-10 (2017).
Pawlikowski et al. Skeletal Muscle, Volume 5, Article number: 42 (2015)
Pitcher et al., (2012). Reduced striatal volumes in Parkinson's disease: a magnetic resonance imaging study. Translational neurodegeneration 1.1 17.
Powers et al., Exercise augmentation of exposure therapy for PTSD: rationale and pilot efficacy data. Cognitive Behaviour Therapy, 44.4, 314-327(2015).
Renault et al., (2009). FoxO3 regulates neural stem cell homeostasis. Cell Stem Cell 5, 527-539.
Rigo et al., Pharmacology of a central nervous system delivered 2′-O-methoxyethyl-modified survival of motor neuron splicing oligonucleotide in mice and nonhuman primates. J. Pharmacol. Exp. Ther. 350, 46-55 (2014).
Rubin et al., Greater hippocampal volume is associated with PTSD treatment response. Psychiatry Research: Neuroimaging 252: 36-39 (2016).
Sanchez-Duffhues et al., Bone morphogenetic protein receptors: Structure, function and targeting by selective small molecule kinase inhibitors. Bone, 138, 115472 (2020).
Sassone et al., Regenerative approaches in Huntington's disease: From mechanistic insights to therapeutic protocols. Frontiers in Neuroscience, 12, p. 800 (2018).
Schoenfeld et al., New neurons restore structural and behavioral abnormalities in a rat model of PTSD. Hippocampus (2019).
Selkoe, Alzheimer disease and aducanumab: adjusting our approach. Nat. Rev. Neurol. 15, 365-366 (2019).
Sorrells et al., Human hippocampal neurogenesis drops sharply in children to undetectable levels in adults. Nature, 555, 377-381 (2018).
Spalding et al., Dynamics of hippocampal neurogenesis in adult humans. Cell 153, 1219-1227 (2013).
Steiner et al., A fresh look at adult neurogenesis. Nature Medicine, 25, 542-543 (2019).
Sterling et al., Striatal shape in Parkinson's disease. Neurobiology of Aging, 34.11, 2510-2516 (2013).
Stiegler, Burden, & Hubbard, Crystal structure of the agrin-responsive immunoglobulin-like domains 1 and 2 of the receptor tyrosine kinase MuSK. J Mol Biol 364: 424-433 (2006).
Tai et al., Neurodegenerative processes in temporal lobe epilepsy with hippocampal sclerosis: Clinical, pathological and neuroimaging evidence. Neuropathology and Applied Neurobiology, 44.1, 70-90 (2018).
Terreros-Roncal et al., Activity-dependent reconnection of adult-born dentate granule cells in a mouse model of frontotemporal dementia. J. Neurosci., 39 (29), 5794-5815 (2019).
Tobin et al., Human hippocampal neurogenesis persists in aged adults and Alzheimer's disease patients. Cell Stem Cell, 24, 974-982.e3 (2019).
van Erp et al., Subcortical brain volume abnormalities in 2028 individuals with schizophrenia and 2540 healthy controls via the ENIGMA consortium. Molecular Psychiatry, 21.4: 547 (2016).
Van et al., Smaller hippocampal volume as a vulnerability factor for the persistence of post-traumatic stress disorder. Psychological Medicine, 45.13: 2737-2746 (2015).
van Praag et al., Exercise enhances learning and hippocampal neurogenesis in aged mice. J. Neurosci., 25, 8680-8685 (2005).
Vaughan et al., Human antibodies by design. Nature Biotechnology, 16, 535-539 (1998).
Villeda et a., The ageing systemic milieu negatively regulates neurogenesis and cognitive function. Nature, 477, 90-94 (2011).
Villeda et al., Young blood reverses age-related impairments in cognitive function and synaptic plasticity in mice. Nature Medicine, 20, 659-663 (2014).
Wang et al., Bone Morphogenetic Protein (BMP) signaling in development and human diseases. Genes Dis. 1, 87-105 (2014).
Watty & Burden, MuSK glycosylation restrains MuSK activation and acetylcholine receptor clustering. J. Biol. Chem. 277, 50457-50462 (2002).
Webb et al., FOXO3 shares common targets with ASCL1 genome-wide and inhibits ASCL1-dependent neurogenesis. Cell Rep., 4, 477-491 (2013).
Whitworth et al., Direct and indirect effects of exercise on posttraumatic stress disorder symptoms: A longitudinal study. General Hospital Psychiatry, 49, 56-62 (2017).
Wilton et a., Antisense oligonucleotide-induced exon skipping across the human dystrophin gene transcript. Mol. Ther., 15, 1288-1296 (2007).
Wolf et al., Klotho up-regulates renal calcium channel transient receptor potential vanilloid 5 (TRPV5) by intra- and extracellular N-glycosylation-dependent mechanisms. J. Biol. Chem. 289, 35849-35857 (2014).
Wu & Nacu, Fast and SNP-tolerant detection of complex variants and splicing in short reads. Bioinformatics, 26, 873-881 (2010).
Wyss-Coray, Ageing, neurodegeneration and brain rejuvenation. Nature, 539, 180-186 (2016).
Yadin, Knaus, & Müller, Structural insights into BMP receptors: Specificity, activation and inhibition. ScienceDirect, Cytokine & Growth Factor Reviews, 27, 13-34 (February 2016).
Yilmaz et al., MuSK is a BMP co-receptor that shapes BMP responses and calcium signaling in muscle cells. Science Signaling, Vol. 9, Issue 444, pp. RA87, pg 1-2 (Sep. 6, 2016). MuSK is a BMP co-receptor. MuSK binds to BMP4 at the Ig3 domain. The ‘Ig3’ domain of MuSK is required for high affinity binding of BMPs. The major species of MuSK expressed endogenously is full length. This Ig3 domain can be alternatively spliced endogenously, creating an isoform termed Δg3MuSK. This splicing entails the coordinated removal of exons 6 and 7 from the MuSK pre mRNA. Yilmaz et al. (2016) immobilized full-length MuSK or ΔIg3 MuSK ectodomain Fc-fusions. These protein constructs were then incubated with BMP4. Yilmaz et al. (2016) showed not only that MuSK binds to BMP but also showed that MuSK binds to BMP receptors. Immobilized MuSK was again incubated with different concentrations of ALK4, ALK6, ALK3, ALK2, BMPRI, ActRIIB, and TROY. MuSK binds best to ALK4 and ALK6. Yilmaz et al. (2016). MuSK is a BMP co-receptor that shapes BMP responses and calcium signaling in muscle cells. Sci. Signal. 9, ra87. Yilmaz discloses a MuSK polypeptide lacking a functional Ig3 domain or reducing level or activity of a BMP-MuSK polypeptide complex, wherein the MuSK comprises a functional Ig3 domain (pg. 2, col. 2, para, 1). To assay the potential role of this domain in MuSK-BMP4 binding, the inventors generated constructs encoding Fe-fusion ectodomain proteins either containing or lacking the Ig3 domain (FL and DIg3, respectively) and assayed their binding to soluble BMP4 over a range of concentrations. MuSK lacking a domain is a mutant. The construct produces MuSK lacking an Ig3 domain, which increases the level and activity of this polypeptide when compared to the wild type). Yilmaz et al. (2016) does not disclose using this in a method of treating a subject suffering from one or more features of neurodegeneration or impaired cognition.
Yun et al., Re-evaluating the link between neuropsychiatric disorders and dysregulated adult neurogenesis. Nature Medicine, 22(11), p. 1239 (2016).
Zhang et al., A role for adult TLX-positive neural stem cells in learning and behaviour. Nature, 451, 1004-1007 (2008b).
Zhang et al., LRP4 serves as a coreceptor of agrin. Neuron, 60, 285-297 (2008a).

### TEXTBOOKS AND TECHNICAL REFERENCES

Current Protocols in Immunology (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
Current Protocols in Molecular Biology (CPMB), (2014). Frederick M. Ausubel (ed.), John Wiley and Sons (ISBN 047150338×, 9780471503385).
Current Protocols in Protein Science (CPPS), (2005). John E. Coligan (ed.), John Wiley and Sons, Inc.
Immunology (2006). Werner Luttmann, published by Elsevier.
Janeway's Immunobiology, (2014). Kenneth Murphy, Allan Mowat, Casey Weaver (eds.), Taylor & Francis Limited, (ISBN 0815345305, 9780815345305).
Laboratory Methods in Enzymology: DNA, (2013). Jon Lorsch (ed.) Elsevier (ISBN 0124199542).
Lewin's Genes XI, (2014). published by Jones & Bartlett Publishers (ISBN-1449659055).
Molecular Biology and Biotechnology: a Comprehensive Desk Reference, (1995). Robert A. Meyers (ed.), published by VCH Publishers, Inc. (ISBN 1-56081-569-8).
Molecular Cloning: A Laboratory Manual, 4th ed., Michael Richard Green and Joseph Sambrook, (2012). Cold Spring Harbor Laboratory Press, Cold Spring Harbor, N.Y., USA (ISBN 1936113414).
The Encyclopedia of Molecular Cell Biology and Molecular Medicine, Robert S. Porter et al. (eds.), published by Blackwell Science Ltd., 1999-2012 (ISBN 9783527600908).
The Merck Manual of Diagnosis and Therapy, 19^{th} edition (Merck Sharp & Dohme Corp., 2018).
Pharmaceutical Sciences 23^{rd} edition (Elsevier, 2020).

### LIST OF EMBODIMENTS

Specific compositions and methods of characterizing the binding interactions between Musk and Bmp receptors were described. The scope of the invention should be defined solely by the claims. A person having ordinary skill in the biomedical art will interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

When the specification provides a range of values, each intervening value between the upper and lower limit of that range is within the range of values unless the context dictates otherwise.

1-6. (canceled)

7. A method of treating a subject suffering from one or more features of neurodegeneration or impaired cognition, the method comprising a step of:
(a) increasing level or activity of a MuSK polypeptide lacking a functional Ig2 domain; or
(b) reducing level or activity of a BMP receptor (ALK)-MuSK polypeptide complex, wherein the MuSK comprises a functional Ig2 domain,
further comprising the step of administering a pharmaceutical composition that increases the altered splicing of MuSK transcripts, wherein the alteration comprises skipping one or more exons in the MuSK Ig2 domain,
wherein the skipped exon is exon 5a of MuSK Ig2 domain;
wherein the skipped exon is exon 5b of MuSK Ig2 domain; or
wherein the skipped exons are exons 5a and 5b of MuSK Ig2 domain.

8. A method of increasing neurogenesis, the method comprising a step of:
(a) increasing level or activity of a MuSK polypeptide lacking a functional Ig2 domain; or
(b) reducing level or activity of a BMP-MuSK polypeptide complex, wherein the MuSK comprises a functional Ig2 domain,
further comprising the step of administering a pharmaceutical composition that increases the altered splicing of MuSK transcripts, wherein the alteration comprises skipping one or more exons in the MuSK Ig2 domain,
wherein the skipped exon is exon 5a of MuSK Ig2 domain;
wherein the skipped exon is exon 5b of MuSK Ig2 domain; or
wherein the skipped exons are exons 5a and 5b of MuSK Ig2 domain.

9. The method of claim 7, further comprising the step of administering a pharmaceutical composition that comprises or delivers a MuSK Ig2 modulator agent.

10-13. (canceled)

14. The method of claim 8, further comprising the step of administering a pharmaceutical composition that comprises or delivers a MuSK Ig2 modulator agent.
